(19) United States
(12) Patent Application Publication
(10) Pub. No.: US 2026/0317795 A1
(43) Pub. Date: Oct. 15, 2026

(54) ENZYMATIC ASYMMETRIC SYNTHESES OF N-ALKYL AMINO ACIDS

(71) Applicants: Hoffmann-La Roche Inc., Little Falls, NJ (US); Genentech, Inc., South San Francisco, CA (US)

(72) Inventors: Carmela MOLINARO, Burlingame, CA (US); Sean M. KELLY, San Mateo, CA (US); Hans IDING, Magden (CH); Patrik Franz STOCKER, Basel (CH); Xin LINGHU, Burlingame, CA (US); Tobias Michael BRUETSCH, Dietikon (CH); Sebastian HEROLD, Bad Saeckingen (DE)

(73) Assignees: Hoffmann-La Roche Inc., Little Falls, NJ (US); Genentech, Inc., South San Francisco, CA (US)

(21) Appl. No.: 19/128,810

(22) PCT Filed: Nov. 8, 2023
(86) PCT No.: PCT/US2023/037019

§ 371 Date: May 9, 2025

Related U.S. Application Data
(60) Provisional application No. 63/383,349, filed on Nov. 11, 2022.

(51) Int. Cl. C12P 13/04; C12N 9/20; C12P 17/12; C12P 41/00

(52) U.S. Cl. CPC C12P 13/04; C12N 9/20; C12P 17/12; C12P 41/005; C12Y 301/01003

(57) ABSTRACT

The subject matter described herein is directed to methods of preparing compounds of general Formula I: or a salt or solvate thereof.

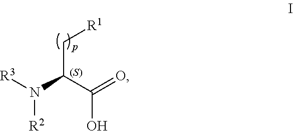

### CROSS-REFERENCE TO RELATED APPLICATIONS

This application claims the benefit of priority to U.S. Provisional Patent Application No. 63/383,349 filed on Nov. 11, 2022, the content of which is incorporated by reference herein in its entirety.

### STATEMENT REGARDING FEDERALLY SPONSORED RESEARCH

This application contains an invention made with Government support under Agreement HHSO100201800036C, awarded by HHS. The Government has certain rights in the invention.

### FIELD

The subject matter described herein is directed to methods useful for preparing enantiomerically enriched N-alkyl amino acids using enzyme-catalyzed dynamic kinetic resolution of N-alkyl amino acid esters via hydrolysis.

### BACKGROUND

It is inefficient to produce optically N-alkyl active amino acids by using a racemic amino acid ester as a raw material. In particular, it is a challenge to obtain optically N-alkyl active amino acids from N-alkyl amino acid esters in high yield and with high enantioselectivity. Yet such N-alkyl amino acids can be building blocks for the chemical synthesis of pharmaceuticals, such as antibiotics, and agrochemicals. For example, the preparation of the ring portion of arylomycin-like compounds can be made from enantiomerically enriched N-alkyl amino acid starting materials. Arylomycin-like compounds have been identified as inhibitors of bacterial signal peptidases and show potential for treatment of infections involving Gram positive and Gram negative bacterial strains that are resistant to existing antibiotics. See, e.g., WO2020/243155 and N. Wong, F. Petronijević, A. Y. Hong, X. Linghu, S. M. Kelly, H. Hou, T. Cravillion, N.-K. Lim, S. J. Robinson, C. Han, C. Molinaro, C. G. Sowell, F. Gosselin, Org. Lett. 2019, 21, 9099-9103.

The subject matter described herein provides methods useful for preparing an optically active carboxylic acid having an α-alkyl nitrogen substituent at a high yield and with high enantioselectivity by using dynamic kinetic resolution (DKR).

### BRIEF SUMMARY

One aspect described herein is a method of preparing an enantiomerically enriched compound of Formula I:

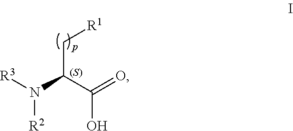

or a salt or solvate thereof,
wherein,
   R¹ is selected from the group consisting of: C₃₋₆ cycloalkyl; C₂₋₆ heterocycloalkyl having 1, 2, or 3 heteroatoms selected from O, S, and N; C₆₋₁₀ aryl; and 5- to 10-membered heteroaryl having 1, 2, or 3 heteroatoms selected from O, S, and N; wherein each can be unsubstituted or substituted with one, two, three, or four substituents independently selected from the group consisting of hydroxyl, —O-PG, —O—C₁₋₆ alkyl, halo, and C₁₋₆ alkyl,
      wherein PG is an oxygen protecting group, and
      wherein the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
      wherein R^{x} and R^{y} are each independently selected from the group consisting of hydrogen, C₁₋₆ alkyl, and a N-protecting group;
   R² is a carbamate N-protecting group;
   R³ is C₁₋₆ alkyl; and
   p is 1 or zero;
the method comprising:
   contacting in an aqueous solution an enantiomeric mixture of Formula I-i:

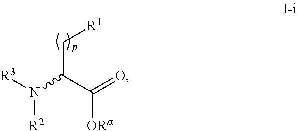

wherein,
R¹, R², R³, and p are as defined in the compound of Formula I; and
R^{a} is C₁₋₆ alkyl or —(CH₂CH₂O)_{q}—C₁₋₆ alkyl, wherein q is 1, 2, 3, 4, or 5;
with a carboxylic ester hydrolase to produce the enantiomerically enriched compound of Formula I, or a salt or solvate thereof.

Other embodiments are also described herein.

### BRIEF DESCRIPTION OF THE FIGURES

FIG. 1 depicts a powder X-ray diffraction pattern of 2ac lysine salt (top).

FIG. 2 depicts a thermogravimetry (TG) thermogram (top) and a differential scanning calorimetry (DSC) thermogram (bottom) of 2ac lysine salt.

FIG. 3 depicts a powder X-ray diffraction pattern of 2ac piperazine salt (top).

FIG. 4 depicts a thermogravimetry (TG) thermogram (top) and a differential scanning calorimetry (DSC) thermogram (bottom) of 2ac piperazine salt.

FIG. 5 depicts a powder X-ray diffraction pattern of 2ac dicyclohexylamine salt.

FIG. 6 depicts a thermogravimetry (TG) thermogram (top) and a differential scanning calorimetry (DSC) thermogram (bottom) of 2ac dicyclohexylamine salt.

### DETAILED DESCRIPTION

The ever growing importance of enantiomerically enriched unnatural amino acids as chiral building blocks used in catalysis, bioactive peptidomimetics drugs, agrochemicals, and semi-synthetic antibiotics has created a need for synthesizing these molecules efficiently. As an illustrative example, enantiomerically enriched building blocks are required for synthesizing arylomycin analogs for therapeutic use as antibiotics. In particular, one of the key challenges presented by these analogues is a practical access to the stereochemically pure unnatural N-alkyl amino acids. The methods described herein address this need.

Several chemo and biocatalytic methods have been reported for the synthesis of enantiomerically enriched unnatural amino acids. Yet the methods require high catalyst loadings, toxic metals, expensive chirality sources, or narrow substrate scope and tend to suffer from low yields and racemization, limiting their practical application in pharmaceutical manufacturing. For example, the enantiomerically enriched unnatural amino acid D-hydroxyphenylglycine can be produced by using (a) two sequential enzymatic reactions catalyzed by D-hydantoinase and N-carbamoylase or (b) a recombinant E. coli strain expressing both enzymes. But these methods are limited in scope. Alternatively, racemic synthesis followed by diastereoisomeric salt crystallization or kinetic resolution can be used, however, the latter approach is limited to a maximum yield of 50% for the desired enantiomer.

Furthermore, access to enantiomerically enriched N-methyl or N-alkyl-arylglycines pose additional challenges, as most of the methods reported suffer from drawbacks such as low chemical yields, narrow substrate scope, and racemization problems. The disclosed methods are based on dynamic kinetic resolution (DKR), driven by continuous in situ rapid epimerization of the starting material, and are useful for asymmetric preparation of N-alkyl amino acid building blocks. These building blocks have nearly limitless uses. With a theoretical yield of 100%, the disclosed methods using DKR provide a solution to overcome the low theoretical yield encountered with classical resolution strategies and alternative access to enantiomerically enriched N-alkyl unnatural amino acids. These methods are useful for the rapid and modular preparation of a broad range of aromatic and aliphatic enantiomerically enriched N-alkyl amino acids, in particular, unnatural amino acids (e.g., hydroxyphenylglycines and N-alkyl-hydroxyphenylglycines), in up to about 99% yield and up to about 99% enantiomerically enriched (ee).

The methods herein are practical, high yielding, and highly enantioselective and comprise carboxylic ester hydrolase-catalyzed DKR of a unique type of substrate N-alkyl amino acid esters, such as PEGylated amino acid esters or alkyl amino acid esters. The carboxylic ester hydrolase, for example, Lipase 147 from Evoxx Technologies GmbH (evo-1.3.147.S), acts as a biocatalyst under basic conditions to produce a broad range of N-alkyl-hydroxyphenylglycines and other unnatural amino acids, as depicted in Scheme 1.

Also described herein is the beneficial finding that the methods are performed under aqueous conditions. Solubilizing the N-alkyl amino acid esters, such as by using PEGylated esters, can confer an improvement over some hydrophobic carboxylic ester hydrolase substrates, allowing for enhanced enzymatic reactivity and scalability. In some embodiments, the PEGylated substrates of the methods of the disclosure have increased solubility compared to some hydrophobic carboxylic ester hydrolase substrates in aqueous media, therefore allowing for a higher reactivity of the enzyme and permitting a significant decrease in carboxylic ester hydrolase loading while still achieving complete conversion. Without wishing to be bound by theory, the increased solubility may facilitate the accessibility of the substrate to the enzyme. The PEGylated esters of the methods of the disclosure may also represent activated esters (like MEM-esters) (Iyer, P. V.; Ananthanarayan, L. Process Biochem. 2008, 43, 1019-1032) with increased enzymatic activity, even at lower pH values (e.g., pH 8.7 versus pH 9.2). These PEGylated esters of the methods of the disclosure may also obviate the need for additional organic solvent to solubilize the substrate. Organic solvents may negatively influence the catalytic activity of the enzyme by changing the enzyme's three-dimensional structure.

A general synthetic route for the methods described herein is depicted in Scheme 1.

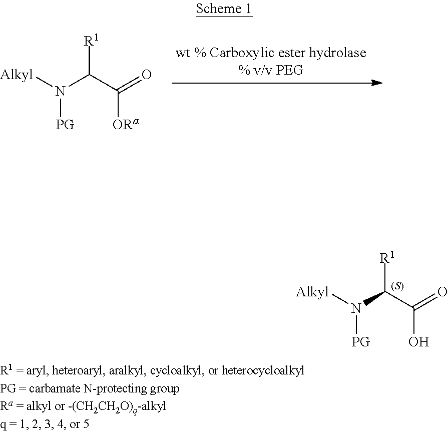

The presently disclosed subject matter will now be described more fully hereinafter. Yet many modifications and other embodiments of the presently disclosed subject matter set forth herein will come to mind to a skilled artisan to which the presently disclosed subject matter pertains having the benefit of the teachings presented in the disclosure. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

Each embodiment described herein may be taken alone or in combination with any one or more other embodiments.

Efforts have been made to ensure accuracy with respect to numbers used (e.g., amounts, temperature, etc.) but some experimental errors and deviations should be accounted for.

### I. Terms

As used in the present specification, the following words, phrases and symbols are generally intended to have the meanings as set forth below, except to the extent that the context in which they are used indicates otherwise.

It is understood that any numerical range recited herein includes all values from the lower value to the upper value. For example, if a range is stated as 1 wt % to 50 wt %, it is intended that values such as 2 wt % to 40 wt %, 10 wt % to 30 wt %, or 1 wt % to 3 wt %, etc., are expressly enumerated in this specification. These are only examples of what is specifically intended, and all possible combinations and subcombinations of ranges and numerical values between and including the lowest value and the highest value enumerated are to be considered to be expressly stated in this application.

“About,” as used herein, when referring to a value is meant to encompass variations of the value. In certain embodiments, “about” includes the indicated value±10%. In other embodiments, “about” includes the indicated value±5%. In certain other embodiments, “about” includes the indicated value±1%. In certain other embodiments, “about” includes the indicated value±0.5%. In certain other embodiments, “about” includes the indicated value±0.1%. Such variations are appropriate to perform the disclosed methods or employ the disclosed compositions. Reference to “about” a value herein also includes (and describes) embodiments that are directed to that value per se. For example, “about x” includes description of “x”.

The singular forms “a,” “an,” and “the” include plural references unless the context clearly dictates otherwise. Thus, for example, reference to “a compound” includes a plurality of such compounds and reference to “the assay” includes reference to one or more assays and equivalents thereof known to the skilled artisan.

Throughout this specification and the claims, the words “including,” “containing,” “having,” and “comprising,” as well as variations thereof, are used in a non-exclusive sense, except where the context requires otherwise. It is understood that embodiments described herein include “consisting of” and/or “consisting essentially of” embodiments.

As described herein, compounds of the present disclosure may be unsubstituted or substituted with one or more substituents, such as those illustrated generally herein, or as exemplified by particular classes, subclasses, and species of the present disclosure. In general, the term “substituted” refers to the replacement of a hydrogen atom in a given structure with a specified substituent. In some embodiments, more than one hydrogen atom is replaced with a specified substituent (e.g. when two hydrogen atoms are replaced with one oxo substituent). Combinations of substituents envisioned by the present disclosure are typically those that result in the formation of stable or chemically feasible compounds.

“Alkyl,” as used herein, refers to an unbranched or branched saturated hydrocarbon chain. In some embodiments, if not otherwise described, alkyl comprises 1 to 12 carbon atoms (C₁-C₁₂alkyl), 1 to 8 carbon atoms (C₁-C₈alkyl), 1 to 6 carbon atoms (C₁-C₆alkyl), or 1 to 4 carbon atoms (C₁-C₄alkyl). Examples of alkyl groups may include methyl, ethyl, n-propyl, isopropyl, n-butyl, sec-butyl, tert-butyl, n-pentyl, 2-pentyl, isopentyl, neopentyl, n-hexyl, 2-hexyl, 3-hexyl, and 3-methyl pentyl.

“Cycloalkyl,” as used herein, may refer to a monocyclic or polycyclic saturated, non-aromatic hydrocarbon. In some embodiments, unless otherwise described, cycloalkyl comprises 3 to 12 carbon atoms (C₃-C₁₂cycloalkyl), 3 to 8 carbon atoms (C₃-C₈cycloalkyl), 3 to 6 carbon atoms (C₃-C₆cycloalkyl), or 3 to 5 carbon atoms (C₃-C₈cycloalkyl). Examples of cycloalkyl groups include cyclopropyl, cyclobutyl, cyclopentyl, cyclohexyl, and the like.

“Heterocycloalkyl,” as used herein, may refer to non-aromatic, saturated monocyclic or polycyclic ring system containing carbon and at least one ring heteroatom. In some embodiments, the heteroatoms are independently selected from N, O, and S. The heterocycloalkyl group may comprise 3, 4, 5, 6, 7, 8, 9, or 10 ring atoms, wherein the ring atoms refer to the sum of carbon and heteroatoms in the one or more rings (e.g., a 5-membered, 6-membered, 7-membered, 8-membered, 9-membered, or 10-membered heterocycloalkyl). In some embodiments, the heterocycloalkyl is a 3- to 6-membered heterocycloalkyl. In some embodiments, the heterocycloalkyl is a 5- to 6-membered heterocycloalkyl. In some embodiments, the heterocycloalkyl is a 6-membered heterocycloalkyl. In some embodiments, the heterocycloalkyl comprises from 2 to 6 carbon atoms (C₂₋₆ heterocycloalkyl). Heterocycloalkyl may include groups comprising 1 to 3 ring heteroatoms, 1 or 2 ring heteroatoms, or 1 ring heteroatom. When the heterocycloalkyl is a polycyclic group, the attachment point to another moiety (e.g., to the rest of a formula) may occur on any ring. Examples of heterocycloalkyl include oxetanyl, azetidinyl, tetrahydrofuranyl, tetrahydropyranyl, pyrrolidinyl, oxazolinyl, oxazolidinyl, thiazolidinyl, pyranyl, thiopyranyl, tetrahydropyranyl, dioxinyl, piperidinyl, morpholinyl, thiomorpholinyl, and piperazinyl.

As used herein, the term “aryl” may refer to a monocyclic or polycyclic group comprising at least one 4n+2 hydrocarbon aromatic ring (e.g., having 6, 10, or 14 π electrons shared in a cyclic array), wherein all of the ring atoms of the at least one hydrocarbon aromatic ring are carbon. Aryl may include groups with a single aromatic ring (e.g., phenyl) and multiple fused aromatic rings (e.g., naphthyl or anthryl). Aryl may further include groups with one or more aromatic hydrocarbon rings fused to one or more non-aromatic hydrocarbon rings. In certain embodiments, aryl includes groups with an aromatic hydrocarbon ring fused to a non-aromatic ring, wherein the non-aromatic ring comprises at least one ring heteroatom independently selected from N, O, and S. In some embodiments, unless otherwise specified, aryl as used herein comprises from 6 to 14 carbon atoms (C₆-C₁₄aryl) or 6 to 10 carbon atoms (C₆-C₁₀aryl). In certain embodiments, each of the aryl groups described herein comprises 6 to 10 carbon atoms. Wherein the aryl is a polycyclic group, the attachment point to another moiety (e.g., to the rest of a formula) may occur on any ring.

“Heteroaryl,” as used herein, may refer to a monocyclic or polycyclic group comprising at least one 4n+2 aromatic ring (e.g., having 6 or 10 π electrons shared in a cyclic array), wherein the aromatic ring comprises at least one ring heteroatom. In some embodiments, the heteroatom is independently selected from N, O, and S. Unless otherwise specified, a heteroaryl group may comprise 5, 6, 7, 8, 9, or 10 ring atoms, where ring atoms refer to the sum of carbon and heteroatoms in the one or more rings (e.g., a 5-membered, 6-membered, 7-membered, 8-membered, 9-membered, or 10-membered heteroaryl). Heteroaryl may also include polycyclic groups with at least one aromatic ring comprising at least one ring heteroatom, fused to a non-aromatic hydrocarbon ring. Heteroaryl may also include polycyclic groups comprising at least one aromatic ring comprising at least one ring heteroatom fused to an aromatic hydrocarbon ring (e.g., quinolinyl, quinoxalinyl, or benzothiazolyl). Heteroaryl may include polycyclic groups with two fused aromatic rings, wherein each ring comprises at least one ring heteroatom (e.g., naphthyridinyl). Wherein the heteroaryl is a polycyclic group, the attachment point to another moiety (e.g., to the rest of a formula) may occur on any ring. Heteroaryl may include groups comprising 1 to 4 ring heteroatoms, 1 to 3 ring heteroatoms, 1 or 2 ring heteroatoms, or 1 ring heteroatom. Examples of heteroaryl moieties include imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, triazolyl, triazinyl, quinoxalinyl, purinyl, quinazolinyl, quinolizinyl, naphthyridinyl, and the like.

As used herein, “halo,” “halogen” and “halide,” which may be used interchangeably, refer to a fluoro, chloro, bromo, or iodo.

“Haloalkyl” refers to an alkyl as defined herein in which one or more hydrogens have been replaced with same or different halogen. Exemplary haloalkyls include —CH₂CI, —CH₂CF₃, —CH₂CCl₃, perfluoroalkyl (e.g., —CF₃), and the like.

As used herein, “dynamic kinetic resolution (DKR)” refers to reaction conditions wherein the chiral center can epimerize so that the (R) and (S) enantiomers can interconvert throughout the reaction process. In the methods described herein, the enzyme selectively lowers the transition state energy of a single enantiomer, as the binding of only one single enantiomer into the enzyme's active site is preferred, leading to the high yield and enantiomeric excess described herein.

As used herein, “protective group” or “protecting group” refer to a group which selectively blocks one reactive site in a multifunctional compound such that a chemical reaction can be carried out selectively at another unprotected reactive site in the meaning conventionally associated with it in synthetic chemistry. Certain processes of this disclosure rely upon the protective groups to block reactive nitrogen and/or oxygen atoms present in the reactants. For example, “amino-protecting group,” “N-protecting group,” and “nitrogen protecting group” are used interchangeably herein and refer to those organic groups intended to protect the nitrogen atom against undesirable reactions during synthetic procedures. Exemplary nitrogen protecting groups include trifluoroacetyl (TFA); acetyl (Ac); benzyl (Bn); tosyl (Ts); trityl (Tr); carbamates such as benzyloxycarbonyl (carbobenzyloxy, Cbz), p-methoxybenzyloxycarbonyl, p-nitrobenzyloxycarbonyl, tert-butoxycarbonyl (Boc), and fluorenylmethyloxycarbonyl (Fmoc); and the like. “Oxygen-protecting group,” “O-protecting group,” and “hydroxyl protecting group” are used interchangeably herein and are used in accordance with their meaning in this field and may refer to those organic groups intended to protect the oxygen atom against undesirable reactions during synthetic procedures. Exemplary oxygen protecting groups include benzyl (Bn), methyl (Me), t-butyldimethylsilyl (TBDMS), and trimethylsilylethoxymethyl (SEM). Exemplary oxygen protecting groups also include p-methylbenzyl and p-phenylbenzyl. The skilled artisan will know how to choose a group for the ease of removal and for the ability to withstand the disclosed reactions.

“Contacting” and “reacting” when referring to a chemical reaction may refer to adding or mixing two or more reagents under appropriate conditions to produce the indicated and/or the desired product. It should be appreciated that the reaction which produces the indicated and/or the desired product may not necessarily result directly from the combination of two reagents which were initially added, e.g., there may be one or more intermediates which are produced in the mixture which ultimately leads to the formation of the indicated and/or the desired product.

As used herein, the abbreviation “PEG” refers to poly(ethylene glycol), also known as poly(ethylene oxide), abbreviated PEO. As a co-solvent, in its most common form, PEG is a linear polymer terminated at each end with hydroxyl groups: HO—CH₂CH₂O—(CH₂CH₂O)ₙ—CH₂CH₂—OH, wherein n is an integer. The polymer, alpha-,omega-dihydroxylpoly(ethylene glycol), can be represented in brief form as “HO-PEG-OH” where it is understood that the “PEG” symbol represents the following structural unit: —CH₂CH₂O—(CH₂CH₂O)ₙ—CH₂CH₂—, wherein n is an integer typically ranging from about 3 to about 4000, or the value of n is such that the average molecular weight of the polymer is from about 200 kDa to about 6,000 kDa. Polymers are designated PEG200, PEG250, PEG400, PEG4000 and PEG6000 and the like. However, when a “PEG” is covalently bound and forms a moiety on a compound described herein, its chemical structure is generally —(CH₂CH₂O)_{q}-alkyl, wherein q and alkyl are as described herein. In some embodiments, alkyl is methyl. In some embodiments, q is 1. In some embodiments, q is 2. In some embodiments, q is 3.

As used herein, “aqueous solution” is used in accordance with its meaning in this field. “Aqueous solution” may refer to a solution using water as solvent, wherein no co-solvent is added to that specific solution. “Aqueous solution” may also refer to water-based solutions containing one or more co-solvents.

“Solvent,” as used herein, refers to a substance such as a liquid or a miscible, partially miscible or immiscible mixture of two or more liquids, which is capable of completely dissolving, partially dissolving, dispersing, or partially dispersing another substance (e.g., a solid or a liquid). “Organic solvent” means an organic material that is a liquid and is capable of dissolving other substances. Solvent and organic solvent as used herein may refer to mixtures of two or more solvents or organic solvents. Suitable solvents that can be used include water and any organic solvents from the various classes of solvents, such as, for example, alcohols, amines, ketones, esters, ethers, halogenated hydrocarbons or chlorinated organic solvents, aromatic hydrocarbons, nitriles, aprotic polar solvents, polar protic solvents, acidic solvents, non-polar solvents, and mixtures of any two or more thereof. Useful alcohols include, for example, methanol, ethanol, denatured spirits, n-propanol, isopropanol, n-butanol, isobutanol, t-butanol, polyhydroxy alcohols (e.g., ethylene glycol, glycerin (i.e., glycerol), propylene glycol, polyethylene glycol, diethylene glycol, diglycerin, triethylene glycol, tetraethylene glycol, and trimethylolpropane), and the like. Useful amines include trimethylamine and triethylamine. Useful ketones include acetone, propanone, 2-butanone, and the like. Useful chlorinated organic solvents or halogenated hydrocarbons include, for example, dichloromethane, 1,2-dichloroethane, chloroform, carbon tetrachloride, chlorobenzene, and the like. Useful esters include, for example, ethyl acetate, n-propyl acetate, isopropyl acetate, n-butyl acetate, t-butyl acetate, and the like. Useful ethers include, for example, dimethyl ether, diethyl ether, methyl t-butyl ether, ethyl methyl ether, diisopropyl ether, tetrahydrofuran, dioxane, and the like. Useful aromatic hydrocarbons include, for example, toluene, xylene, chlorobenzene, and the like. Useful nitriles include acetonitrile, propionitrile, and the like. Useful “aprotic polar solvents” include THF (tetrahydrofuran); 2-MeTHF (2-methyltetrahydrofuran); dimethylsulfoxide (DMSO); N,N-dimethylacetamide (DMA); DME (dimethoxyethane); MTBE (methyl tert-butyl ether); cyclopentyl methyl ether (CPME); dioxane; chlorinated solvents such as dichloromethane and 1,2-dichloroethane; nitriles such as acetonitrile; ketones such as acetone, methyl ethyl ketone, and methyl isobutyl ketone; amides such as DMF (dimethylformamide), DMAC (dimethylacetamide), and NMP (N-methylpyrrolidone); and the like. Useful “polar protic solvents” include alcohols, such as methanol, ethanol, denatured spirits, n-propanol, isopropanol, n-butanol, isobutanol, or t-butanol. Useful acidic solvents include formic acid, acetic acid, and the like. Useful non-polar solvents include cyclohexanes; heptane; hexanes; esters such as ethyl acetate or butyl acetate; and the like. This listing is not intended to be exhaustive, and combinations of solvents that may be useful can include more than one member of a class, and/or can be from different classes.

As used herein, “co-solvent” means an additional solvent used in the aqueous reaction system. Not all organic solvents are contemplated as co-solvents in the reaction systems described herein. In some embodiments, the co-solvent is soluble or mostly soluble in water. Example co-solvents include DMSO; PEG, such as PEG200, PEG400, and PEG6000; MeOH; EtOH; iPrOH; BuOH; CH₃CN; acetone; tBuOH; nPrOH; ethylene glycol; trimethylamine; 2-MeTHF; heptane; cyclohexanes; glycerol; and THE, or combinations of any of the foregoing.

In some embodiments of the disclosure, the concentration of the enantiomeric mixture of Formula I-i in the aqueous solution may be provided in mL/g. For example, if the concentration of the enantiomeric mixture of Formula I-i in the aqueous solution is 15 mL/g, this means that there are 15 mL of aqueous solution per 1 gram of the enantiomeric mixture of Formula I-i.

References to wt % as used herein are based upon the weight of amino acid ester substrate used in the method. In other words, the weight of amino acid ester substrate is the standard used to determine the wt % of other method components. Accordingly, one equivalent of amino acid ester substrate—based upon weight—is present in the methods of the disclosure and all other parameters are calculated versus that reference. For example, if 5 g of PEGylated amino acid ester substrate is used in a method, an enzyme loading of 5 wt % would equal 0.25 g of enzyme.

In some embodiments of the disclosure, the concentration of the co-solvent in the aqueous solution may be provided as % v/v. For example, if the aqueous solution has a volume of 100 mL and there are 20 mL of PEG400 present in the aqueous solution, the % v/v of PEG400 in the aqueous solution is 20%.

In some embodiments, the method of the disclosure does not comprise a chromatographic purification or only minimal chromatography is used (e.g., one chromatographic purification). As used herein, chromatographic purification and column chromatography refers to the separation of bulk substances based on differential adsorption of compounds to the adsorbent in a column, where compounds move through the column at different rates, which allows different compounds to be separated into fractions.

“C₁₋₄ alcohol” refers to C₁₋₄ alkyl groups wherein one or two hydrogen atoms are substituted by OH radicals. Non-limiting examples include: methanol, ethanol, n-propanol, isopropanol, n-butanol, iso-butanol, sec-butanol, and tert-butanol.

As used herein, “base” refers to a compound that can accept a proton or donate a lone electron pair. Examples of bases to be used in the aqueous solution to keep the targeted reaction pH include alkali hydroxide (e.g., LiOH, NaOH, KOH, RbOH, and CsOH), carbonate, bicarbonate, phosphate (PO₄²⁻), and the like.

The enantiomerically enriched compounds of Formula I of the disclosure may exist as a salt. Such salts may be prepared by any suitable method, for example, treatment of the free acid with an inorganic or organic base (e.g., if the compound of Formula (I) is a free acid). Salts include, for example, those derived from organic bases (such as an amine (e.g., a primary, secondary or tertiary amine)), an alkali metal hydroxide, alkaline earth metal hydroxide, or the like. Illustrative examples of salts include, but are not limited to, organic salts derived from amino acids (such as lysine, glycine, or arginine); ammonia; primary, secondary, and tertiary amines (e.g., dicyclohexylamine); cyclic amines (such as piperidine, morpholine, and piperazine); and inorganic salts derived from sodium, calcium, potassium, magnesium, manganese, iron, copper, zinc, aluminum, or lithium (such as derived from inorganic bases such as sodium carbonate, sodium hydroxide, calcium hydroxide, potassium hydroxide, aluminum hydroxide, and the like). In some embodiments, the salt of the enantiomerically enriched compound of Formula I is a lysine, piperazine, or dicyclohexylamine salt. In some embodiments, the salt of the enantiomerically enriched compound of Formula I is a lysine salt. In some embodiments, the salt of the enantiomerically enriched compound of Formula I is a piperazine salt. In some embodiments, the salt of the enantiomerically enriched compound of Formula I is a dicyclohexylamine salt.

The enantiomerically enriched compounds of Formula I of the disclosure may exist as solvates. The term “solvate” may refer to a complex of variable stoichiometry formed by a solute and solvent. Such solvents for the purpose of the disclosure may not interfere with the biological activity of the solute. Examples of suitable solvents include, but are not limited to, water, MeOH, and EtOH. Solvates wherein water is the solvent molecule are typically referred to as hydrates. Hydrates may include compositions containing stoichiometric amounts of water, as well as compositions containing variable amounts of water.

The disclosure provides for solid forms of the enantiomerically enriched compounds of Formula I, or salts or solvates thereof, of the disclosure. The term “solid form” may refer to a physical form which is not predominantly in a liquid or a gaseous state. A solid form may be an amorphous form, a crystalline form, or a mixture thereof. In certain embodiments, a solid form may be a liquid crystal. In one embodiment, a lysine salt of the enantiomerically enriched compound of Formula I is a solid form. In another embodiment, a piperazine salt of the enantiomerically enriched compound of Formula I is a solid form. In another embodiment, a dicyclohexylamine salt of the enantiomerically enriched compound of Formula I is a solid form. A solid form may be a crystal form as defined herein.

The solid forms of the enantiomerically enriched compounds of Formula I, or salts or solvates thereof, of the disclosure may be crystalline forms. The term “crystal form” or “crystalline form” may refer to a solid form that is crystalline. In certain embodiments, a crystal form described herein is pure. In certain such embodiments, a crystal form of an enantiomerically enriched compound of Formula I, or a salt or solvate thereof, may be substantially free of amorphous solids and/or other crystal forms. In certain embodiments, a crystal form of an enantiomerically enriched compound of Formula I, or a salt or solvate thereof, may contain less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, or less than about 50% by weight of one or more amorphous solids and/or other crystal forms. In certain embodiments, a crystal form of an enantiomerically enriched compound of Formula I, or a salt or solvate thereof, may be about 99%, about 98%, about 97%, about 96%, about 95%, about 94%, about 93%, about 92%, about 91%, or about 90% pure. In one embodiment, a lysine salt of the enantiomerically enriched compound of Formula I is crystalline. In another embodiment, a piperazine salt of the enantiomerically enriched compound of Formula I is crystalline. In another embodiment, a dicyclohexylamine salt of the enantiomerically enriched compound of Formula I is crystalline.

The solid forms of the enantiomerically enriched compounds of Formula I, or a salt or solvate thereof, of the disclosure may be amorphous solids. The term “amorphous” or “amorphous solid” may refer to a solid form that not substantially crystalline as determined by X-ray diffraction. In particular, the term “amorphous solid” may describe a disordered solid form, i.e., a solid form lacking long range crystalline order. In one embodiment, a lysine salt of the enantiomerically enriched compound of Formula I is amorphous. In another embodiment, a piperazine salt of the enantiomerically enriched compound of Formula I is amorphous. In another embodiment, a dicyclohexylamine salt of the enantiomerically enriched compound of Formula I is amorphous.

Additional terms may be provided below as appropriate.

### II. Asymmetric Syntheses of Amino Acids

In some embodiments, the subject matter described herein is directed to methods of preparing enantiomerically enriched amino acids. In certain embodiments, the amino acid is an unnatural amino acid. In certain embodiments, the yield of the methods is up to about 99%. In certain embodiments, the methods provide up to about 99% enantiomer enrichment (ee).

In certain embodiments, the subject matter described herein is directed to a method of preparing an enantiomerically enriched compound of Formula I:

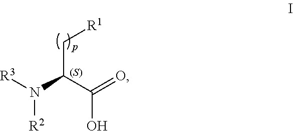

or a salt or solvate thereof,
wherein,
   R¹ is selected from the group consisting of: C₃₋₆ cycloalkyl; C₂₋₆ heterocycloalkyl having 1, 2, or 3 heteroatoms selected from O, S, and N; C₆₋₁₀ aryl; and 5- to 10-membered heteroaryl having 1, 2, or 3 heteroatoms selected from O, S, and N; wherein each can be unsubstituted or substituted with one, two, three, or four substituents independently selected from the group consisting of hydroxyl, —O-PG, —O—C₁₋₆ alkyl, halo, and C₁₋₆ alkyl,
      wherein PG is an oxygen protecting group, and
      wherein the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
      wherein R^{x} and R^{y} are each independently selected from the group consisting of hydrogen, C₁₋₆ alkyl, and a N-protecting group;
   R² is a carbamate N-protecting group;
   R³ is C₁₋₆ alkyl; and
   p is 1 or zero;
the method comprising:
   contacting in an aqueous solution an enantiomeric mixture of Formula I-i:

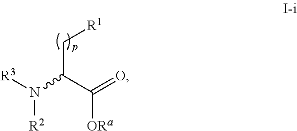

wherein,
R¹, R², R³, and p are as defined in the compound of Formula I; and
R^{a} is C₁₋₆ alkyl or —(CH₂CH₂O)_{q}—C₁₋₆ alkyl, wherein q is 1, 2, 3, 4, or 5;
   with a carboxylic ester hydrolase to produce the enantiomerically enriched compound of Formula I, or a salt or solvate thereof.

In some embodiments, R^{a} is C₁₋₆ alkyl. In certain such embodiments, the enantiomeric mixture of Formula I-i is:

### Formula I-i

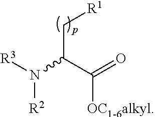

In certain such embodiments, p is 0.

In some embodiments, R^{a} is C₂₋₆ alkyl. In some embodiments, R^{a} is C₂₋₅ alkyl. In some embodiments, R^{a} is methyl, ethyl, n-propyl, n-butyl, n-pentyl, or n-hexyl. In some embodiments, R^{a} is ethyl, n-propyl, n-butyl, n-pentyl, or n-hexyl. In some embodiments, R^{a} is ethyl, n-propyl, n-butyl, or n-pentyl.

In some embodiments, R^{a} is methyl. In certain such embodiments, the enantiomeric mixture of Formula I-i is:

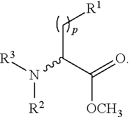

In certain such embodiments, p is 0.

In some embodiments, R^{a} is ethyl. In certain such embodiments, the enantiomeric mixture of Formula I-i is:

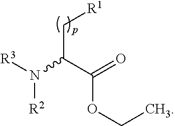

In certain such embodiments, p is 0.

In some embodiments, R^{a} is n-propyl. In certain such embodiments, the enantiomeric mixture of Formula I-i is:

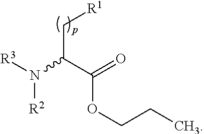

In certain such embodiments, p is 0.

In some embodiments, R^{a} is n-butyl. In certain such embodiments, the enantiomeric mixture of Formula I-i is:

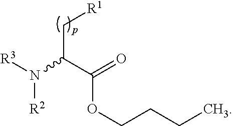

In certain such embodiments, p is 0.

In some embodiments, R^{a} is n-pentyl. In certain such embodiments, the enantiomeric mixture of Formula I-i is:

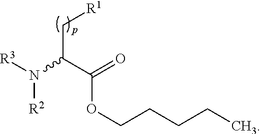

In certain such embodiments, p is 0.

In some embodiments, R^{a} is n-hexyl. In certain such embodiments, the enantiomeric mixture of Formula I-i is:

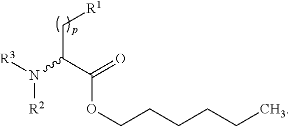

In certain such embodiments, p is 0.

In some embodiments, R^{a} is —(CH₂CH₂O)_{q}—C₁₋₆ alkyl. In certain such embodiments, the enantiomeric mixture of Formula I-i is: Formula I-i

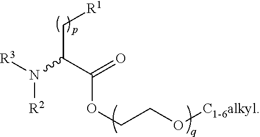

In certain such embodiments, p is 0.

In some embodiments, R^{a} is —(CH₂CH₂O)_{q}—CH₃. In certain such embodiments, the enantiomeric mixture of Formula I-i is: Formula I-i

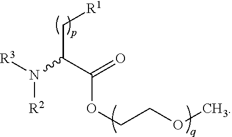

In certain such embodiments, p is 0.

In some embodiments, R^{a} is —(CH₂CH₂O)_{q}—CH₂CH₃. In certain such embodiments, the enantiomeric mixture of Formula I-i is: Formula I-i

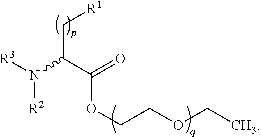

In certain such embodiments, p is 0.

In certain embodiments wherein R^{a} is —(CH₂CH₂O)_{q}—C₁₋₆ alkyl, q is 1, 2, 3, 4, or 5.

In certain embodiments wherein R^{a} is —(CH₂CH₂O)_{q}—CH₃, q is 1, 2, 3, 4, or 5. In certain embodiments wherein R^{a} is —(CH₂CH₂O)_{q}—CH₂CH₃, q is 1, 2, 3, 4, or 5. In certain embodiments, q is an integer from 1 to 3. In certain embodiments, q is 2 or 3. In certain embodiments, q is 1. In certain embodiments, q is 2. In certain embodiments, q is 3. In certain embodiments, q is 1, 2, 3, 4, or 5. In some embodiments, q is 1, 2, or 3.

In some embodiments, R¹ is unsubstituted C₃₋₆ cycloalkyl; unsubstituted C₂₋₆ heterocycloalkyl having 1, 2, or 3 heteroatoms selected from O, S, and N; unsubstituted C₆₋₁₀ aryl; or unsubstituted 5- to 10-membered heteroaryl having 1, 2, or 3 heteroatoms selected from O, S, and N. In some embodiments, R¹ is substituted C₃₋₆ cycloalkyl; substituted C₂₋₆ heterocycloalkyl having 1, 2, or 3 heteroatoms selected from O, S, and N; substituted C₆₋₁₀ aryl; or substituted 5- to 10-membered heteroaryl having 1, 2, or 3 heteroatoms selected from O, S, and N.

In some embodiments, R¹ is unsubstituted or substituted C₃₋₆ cycloalkyl or unsubstituted or substituted C₆₋₁₀ aryl. In some embodiments, R¹ is unsubstituted C₃₋₆ cycloalkyl or unsubstituted C₆₋₁₀ aryl. In some embodiments, R¹ is substituted C₃₋₆ cycloalkyl or substituted C₆₋₁₀ aryl.

In some embodiments, R¹ is unsubstituted or substituted C₃₋₆ cycloalkyl or unsubstituted or substituted C₆ aryl. In some embodiments, R¹ is unsubstituted C₃₋₆ cycloalkyl or unsubstituted C₆ aryl. In some embodiments, R¹ is substituted C₃₋₆ cycloalkyl or substituted C₆ aryl.

In some embodiments, R¹ is unsubstituted or substituted C₃₋₆ cycloalkyl. In some embodiments, R¹ is unsubstituted or substituted C₂₋₆ heterocycloalkyl having 1, 2, or 3 heteroatoms selected from O, S, and N. In some embodiments, R¹ is unsubstituted or substituted C₆₋₁₀ aryl. In some embodiments, R¹ is unsubstituted or substituted C₆ aryl. In some embodiments, R¹ is unsubstituted or substituted 5- to 10-membered heteroaryl having 1, 2, or 3 heteroatoms selected from O, S, and N. In some embodiments, R¹ is unsubstituted C₃₋₆ cycloalkyl. In some embodiments, R¹ is unsubstituted C₂₋₆ heterocycloalkyl having 1, 2, or 3 heteroatoms selected from O, S, and N. In some embodiments, R¹ is unsubstituted C₆₋₁₀ aryl. In some embodiments, R¹ is unsubstituted C₆ aryl. In some embodiments, R¹ is unsubstituted 5- to 10-membered heteroaryl having 1, 2, or 3 heteroatoms selected from O, S, and N. In some embodiments, R¹ is substituted C₃₋₆ cycloalkyl. In some embodiments, R¹ is substituted C₂₋₆ heterocycloalkyl having 1, 2, or 3 heteroatoms selected from O, S, and N. In some embodiments, R¹ is substituted C₆₋₁₀ aryl. In some embodiments, R¹ is substituted C₆ aryl. In some embodiments, R¹ is substituted 5- to 10-membered heteroaryl having 1, 2, or 3 heteroatoms selected from O, S, and N.

In some embodiments, R¹ is unsubstituted or substituted phenyl. In some embodiments, R¹ is unsubstituted phenyl. In some embodiments, R¹ is substituted phenyl. In some embodiments, R¹ is unsubstituted or substituted 5- to 6-membered heteroaryl having 1, 2, or 3 heteroatoms selected from O, S, and N. In some embodiments, R¹ is unsubstituted 5- to 6-membered heteroaryl having 1, 2, or 3 heteroatoms selected from O, S, and N. In some embodiments, R¹ is substituted 5- to 6-membered heteroaryl having 1, 2, or 3 heteroatoms selected from O, S, and N.

In some embodiments, R¹ is unsubstituted or substituted pyridyl. In some embodiments, R¹ is unsubstituted pyridyl. In some embodiments, R¹ is substituted pyridyl.

In some embodiments, R¹ is substituted with one, two, three or four-O-PG, wherein each PG is independently selected from SEM, benzyl, and Me. In some embodiments, at least one PG is benzyl. In some embodiments, at least one PG is Me. In some embodiments, at least one PG is SEM. In some embodiments, R¹ is substituted with one, two, three or four-O-PG, wherein each PG is independently selected from —O-p-methylbenzyl and —O-p-phenylbenzyl. In some embodiments, at least one PG is —O-p-methylbenzyl. In some embodiments, at least one PG is —O-p-phenylbenzyl.

In some embodiments, R¹ is substituted with one, two, three or four halo. In some embodiments, R¹ is substituted with two halo. In some embodiments, R¹ is substituted with two bromo. In some embodiments, R¹ is substituted with one halo. In some embodiments, R¹ is substituted with one bromo. In some embodiments, R¹ is substituted with one iodo. In some embodiments, R¹ is substituted with one chloro. In some embodiments, R¹ is substituted with one fluoro.

In some embodiments, R¹ is substituted with one, two, three or four hydroxyl. In some embodiments, R¹ is substituted with one hydroxyl. In some embodiments, R¹ is substituted with two hydroxyl.

In some embodiments, R¹ is substituted with unsubstituted or substituted C₁₋₆ alkyl and hydroxyl. In some embodiments, R¹ is substituted with methyl and hydroxyl.

In some embodiments, R¹ is substituted with —O-PG and halo. In some embodiments, R¹ is substituted with bromo and —O-Bn.

In some embodiments, R¹ is substituted with halo and hydroxyl. In some embodiments, R¹ is substituted with fluoro and hydroxyl. In some embodiments, R¹ is substituted with iodo and hydroxyl. In some embodiments, R¹ is substituted with chloro and hydroxyl. In some embodiments, R¹ is substituted with bromo and hydroxyl.

In some embodiments, R¹ is substituted with halo, —O-PG, and hydroxyl. In some embodiments, R¹ is substituted with bromo, —O-Bn, and hydroxyl. In some embodiments, R¹ is substituted with iodo, —O-Bn, and hydroxyl. In some embodiments, R¹ is substituted with bromo, —O-SEM, and hydroxyl. In some embodiments, R¹ is substituted with iodo, —O-SEM, and hydroxyl. In some embodiments, R¹ is substituted with bromo, —O-p-methylbenzyl, and hydroxyl. In some embodiments, R¹ is substituted with bromo, —O-p-phenylbenzyl, and hydroxyl.

In some embodiments, R¹ is substituted with halo, unsubstituted or substituted —O—C₁₋₆ alkyl, and hydroxyl. In certain such embodiments, the —O—C₁₋₆ alkyl is substituted with hydroxyl and —NR^{x}R^{y}. In certain such embodiments, R^{x} is H and R^{y} is an N-protecting group. In certain such embodiments, the N-protecting group is Boc. In some embodiments, halo is iodo. In some embodiments, R¹ is substituted with hydroxyl, iodo, and —O—C₁₋₆ alkyl substituted with hydroxyl and —NR^{x}R^{y}. In certain such embodiments, R^{x} is H and R^{y} is an N-protecting group. In certain such embodiments, the N-protecting group is Boc.

In certain embodiments, R³ is methyl, ethyl, n-propyl, isopropyl, n-butyl, sec-butyl, tert-butyl, n-pentyl, 2-pentyl, isopentyl, neopentyl, n-hexyl, 2-hexyl, 3-hexyl, or 3-methyl pentyl. In certain embodiments, R³ is methyl, ethyl, n-propyl, n-butyl, n-pentyl or n-hexyl. In certain embodiments, R³ is methyl. In certain embodiments, R³ is n-butyl.

In certain embodiments, R² is Fmoc, Boc, or Cbz. In certain embodiments, R² is Boc or Cbz. In certain embodiments, R² is Boc. In certain embodiments, R² is Cbz.

In certain embodiments, R¹ is:

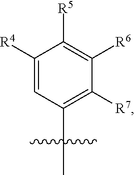

wherein, R⁴, R⁵, R⁶ and R⁷, are each independently selected from the group consisting of hydrogen, hydroxyl, —O-PG, —O—C₁₋₆ alkyl, halo, and C₁₋₆ alkyl,
wherein PG is an oxygen protecting group, and
wherein, the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
   wherein, R^{x} and R^{y} are each independently selected from the group consisting of hydrogen, C₁₋₆ alkyl, and a N-protecting group. In certain such embodiments, p is 0.

In certain embodiments, R⁴, R⁵, R⁶ and R⁷ are each hydrogen. In certain such embodiments, p is zero. In some embodiments wherein R⁴, R⁵, R⁶ and R⁷ are each hydrogen, p is one.

In certain embodiments, R⁴ is hydrogen or halo. In certain embodiments, R⁴ is bromo. In certain embodiments, R⁴ is hydrogen. In some embodiments, R⁴ is —O-PG. In some embodiments, R⁴ is —O-Bn or —O-SEM. In some embodiments, R⁴ is —O-Bn. In some embodiments, R⁴ is —O-SEM. In some embodiments, R⁴ is unsubstituted or substituted —O—C₁₋₆ alkyl. In some embodiments, R⁴ is —O—C₁₋₆ alkyl substituted with hydroxyl and —NR^{x}R^{y}. In certain such embodiments, R^{x} is H and R^{y} is an N-protecting group. In certain such embodiments, the N-protecting group is Boc.

In certain embodiments, R⁵ is hydrogen or hydroxyl. In some embodiments, R⁵ is —O-PG. In some embodiments, R⁵ is —O-Bn or —O-SEM. In some embodiments, R⁵ is —O-Bn. In some embodiments, R⁵ is —O-SEM. In some embodiments, R⁵ is hydroxyl. In some embodiments, R⁵ is hydrogen. In some embodiments, R⁵ is —O-p-methylbenzyl. In some embodiments, R⁵ is —O-p-phenylbenzyl.

In certain embodiments, R⁶ is hydrogen, hydroxyl, halo, or unsubstituted or substituted C₁₋₆ alkyl. In certain embodiments, R⁶ is iodo, bromo, fluoro, or chloro. In certain embodiments, R⁶ is bromo. In certain embodiments, R⁶ is methyl. In some embodiments, R⁶ is —O-PG. In some embodiments, R⁶ is —O-Bn or —O-SEM. In some embodiments, R⁶ is —O-Bn. In some embodiments, R⁶ is —O-SEM. In some embodiments, R⁶ is —O—C₁₋₆ alkyl substituted with hydroxyl and —NR^{x}R^{y}, wherein R^{x} is H and R^{y} is an N-protecting group. In certain such embodiments, the N-protecting group is Boc. In some embodiments, R⁶ is hydrogen.

In certain embodiments, R⁷ is hydrogen, halo, or unsubstituted or substituted C₁₋₆ alkyl. In certain embodiments, R⁷ is fluoro. In certain embodiments, R⁷ is methyl. In some embodiments, R⁷ is hydrogen.

In some embodiments, R⁴ is —O-PG, R⁵ is hydroxyl, R⁶ is halo, and R⁷ is hydrogen. In some embodiments, R⁴ is —O-Bn, R⁵ is hydroxyl, R⁶ is iodo, and R⁷ is hydrogen.

In some embodiments, R⁴ is hydroxyl, R⁵ is —O-PG, R⁶ is halo, and R⁷ is hydrogen. In some embodiments, R⁴ is hydroxyl, R⁵ is —O-SEM, R⁶ is iodo, and R⁷ is hydrogen. In some embodiments, R⁴ is hydroxyl, R⁵ is —O-Bn, R⁶ is iodo, and R⁷ is hydrogen. In some embodiments, R⁴ is hydroxyl, R⁵ is —O-Bn, R⁶ is bromo, and R⁷ is hydrogen. In some embodiments, R⁴ is hydroxyl, R⁵ is —O-SEM, R⁶ is bromo, and R⁷ is hydrogen. In some embodiments, R⁴ is hydroxyl, R⁵ is —O-p-methylbenzyl, R⁶ is bromo, and R⁷ is hydrogen. In some embodiments, R⁴ is hydroxyl, R⁵ is —O-p-phenylbenzyl, R⁶ is bromo, and R⁷ is hydrogen.

In some embodiments, R⁴ is hydrogen, R⁵ is hydroxyl, R⁶ is halo, and R⁷ is hydrogen. In some embodiments, R⁴ is hydrogen, R⁵ is hydroxyl, R⁶ is bromo, and R⁷ is hydrogen. In some embodiments, R⁴ is hydrogen, R⁵ is hydroxyl, R⁶ is fluoro, and R⁷ is hydrogen. In some embodiments, R⁴ is hydrogen, R⁵ is hydroxyl, R⁶ is chloro, and R⁷ is hydrogen. In some embodiments, R⁴ is hydrogen, R⁵ is hydroxyl, R⁶ is iodo, and R⁷ is hydrogen.

In some embodiments, R⁴ is hydrogen, R⁵ is hydroxyl, R⁶ is unsubstituted or substituted C₁₋₆ alkyl, and R⁷ is hydrogen. In some embodiments, R⁴ is hydrogen, R⁵ is hydroxyl, R⁶ is methyl, and R⁷ is hydrogen.

In some embodiments, R⁴ is hydroxyl, R⁵ is hydroxyl, R⁶ is halo, and R⁷ is hydrogen. In some embodiments, R⁴ is hydroxyl, R⁵ is hydroxyl, R⁶ is bromo, and R⁷ is hydrogen.

In some embodiments, R⁴ is halo, R⁵ is hydroxyl, R⁶ is halo, and R⁷ is hydrogen. In some embodiments, R⁴ is bromo, R⁵ is hydroxyl, R⁶ is bromo, and R⁷ is hydrogen.

In some embodiments, R⁴ is hydrogen, R⁵ is hydroxyl, R⁶ is hydrogen, and R⁷ is unsubstituted or substituted C₁₋₆ alkyl. In some embodiments, R⁴ is hydrogen, R⁵ is hydroxyl, R⁶ is hydrogen, and R⁷ is methyl.

In some embodiments, R⁴ is hydrogen, R⁵ is hydroxyl, R⁶ is hydrogen, and R⁷ is halo. In some embodiments, R⁴ is hydrogen, R⁵ is hydroxyl, R⁶ is hydrogen, and R⁷ is fluoro.

In some embodiments, R⁴ is hydrogen, R⁵ is hydrogen, R⁶ is halo, and R⁷ is hydrogen. In some embodiments, R⁴ is hydrogen, R⁵ is hydrogen, R⁶ is bromo, and R⁷ is hydrogen.

In some embodiments, R⁴ is hydrogen, R⁵ is —O-PG, R⁶ is halo, and R⁷ is hydrogen. In some embodiments, R⁴ is hydrogen, R⁵ is —O-Bn, R⁶ is bromo, and R⁷ is hydrogen.

In some embodiments, R⁴ is hydrogen, R⁵ is hydroxyl, R⁶ is hydrogen, and R⁷ is hydrogen.

In some embodiments, R⁴ is substituted —O—C₁₋₆ alkyl, R⁵ is hydroxyl, R⁶ is halo, and R⁷ is hydrogen. In certain such embodiments, the —O—C₁₋₆ alkyl is substituted with hydroxyl and —NR^{x}R^{y}, wherein R^{x} is H and R^{y} is an N-protecting group. In certain such embodiments, the N-protecting group is Boc. In some embodiments, R⁴ is substituted —O—C₁₋₆ alkyl, R⁵ is hydroxyl, R⁶ is iodo, and R⁷ is hydrogen. In certain such embodiments, the —O—C₁₋₆ alkyl is substituted with hydroxyl and —NR^{x}R^{y}, wherein R^{x} is H and R^{y} is an N-protecting group. In certain such embodiments, the N-protecting group is Boc. In some embodiments, R⁴ is substituted —O—C₁₋₆ alkyl, R⁵ is hydroxyl, R⁶ is bromo, and R⁷ is hydrogen. In certain such embodiments, the —O—C₁₋₆ alkyl is substituted with hydroxyl and —NR^{x}R^{y}, wherein R^{x} is H and R^{y} is an N-protecting group. In certain such embodiments, the N-protecting group is Boc.

In certain embodiments, p is one. In certain embodiments, p is zero.

In certain embodiments, R¹ is:

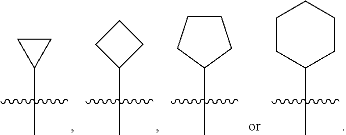

In some embodiments, the carboxylic ester hydrolase, sometimes described as a lipase, is a cholesterol esterase (EC 3.1.1.13), a triacylglycero-protein acylhydrolase (EC 3.1.1.34), triacylglycerol lipase (EC 3.1.1.3), or an aminoacylase (EC 3.5.1.14). In some embodiments, the carboxylic ester hydrolase is a triacylglycerol lipase (EC 3.1.1.3). In certain embodiments, the carboxylic ester hydrolase is selected from Fluka, acylase from Streptomyces toyocaensis, 94734 (EC 3.5.1.14; CAS No. 9012-37-7; aminoacylase); Sorachim SA, cholesterol esterase from microorganism, COE-313 (EC 3.1.1.13; cholesterol esterase); Toyobo Co., Ltd., lipoprotein lipase from Pseudomonas sp., LIP-301 (immobilized) and LPL-311 (EC 3.1.1.34; triacylglycero-protein acylhydrolase; see T. Saiki, Y. Takagi, T. Suzuki, T. Narasaki, G. Tamura and K. Arima, Agric. Biol. Chem. (Tokyo), 33, 414 (1969); see also T. Yamaguchi, N. Muroya, M. Isobe and M. Sugiura, Agric. Biol. Chem. (Tokyo), 37, 999 (1973)); Sarochim SA, cholesterol esterase from Pseudomonas sp., COE-311 (EC 3.1.1.13; cholesterol esterase); Amano Enzyme USA Co., Ltd., esterase from Escherichia coli, CES-E2 (EC 3.1.1.3; triacylglycerol lipase); and Lipase 147, Evoxx Technologies GmbH, evo-1.3.147.S (EC 3.1.1.3; CAS No. 9001-62-1; triacylglycerol lipase). In certain embodiments, the carboxylic ester hydrolase is Lipase 147, Evoxx Technologies GmbH, evo-1.3.147.S.

In some embodiments, the carboxylic ester hydrolase is used in various forms including a purified enzyme, a crude enzyme, a microbial culture, a bacterial cell, and a treated object thereof. Examples of the treated object used herein include a lyophilized bacterial cell, an acetone-dried bacterial cell, a ground bacterial cell, an autodigested substance of bacterial cell, an ultrasonic-treated object of bacterial cell, bacterial cell extract, or an alkaline-treated object of bacterial cell. In other embodiments, the carboxylic ester hydrolase in various purities or forms may be immobilized for use, for example, by known methods including an adsorption method to an inorganic carrier such as silica gel and ceramics, cellulose, ion-exchange resin and so on; a polyacrylamide method; a sulfur-containing polysaccharide gel method (for example, a carrageenan gel method); an alginic acid gel method; an agar gel method and so on. Any means of immobilizing enzymes generally known in the art may be used to immobilize the carboxylic ester hydrolase to a carrier. For example, the carboxylic ester hydrolase may be bound directly to a membrane, granules or the like of a resin having one or more functional groups, or it may be bound to the resin through bridging compounds having one or more functional groups, e.g. glutaraldehyde. Such enzyme immobilizing reactions are described, for example, on pages 369-394 of the 2nd Edition of Microbial Enzymes and Biotechnology (Elsevier Applied Science 1990; Ed. W. M. Fogarty and C. T. Kelly).

In certain embodiments, the carboxylic ester hydrolase is present from about 1 wt % to about 50 wt %. In certain embodiments, the carboxylic ester hydrolase is present from about 1 wt % to about 40 wt %. In certain embodiments, the carboxylic ester hydrolase is present from about 1 wt % to about 30 wt %. In certain embodiments, the carboxylic ester hydrolase is present from about 1 wt % to about 20 wt %. In certain embodiments, the carboxylic ester hydrolase is present from about 1 wt % to about 10 wt %. In certain embodiments, the carboxylic ester hydrolase is present from about 2 wt % to about 8 wt %. In certain embodiments, the carboxylic ester hydrolase is present from about 30 wt % to about 50 wt %. In certain embodiments, the carboxylic ester hydrolase is present from about 40 wt % to about 50 wt %. In certain embodiments, the carboxylic ester hydrolase is present from about 20 wt % to about 40 wt %. In certain embodiments, the carboxylic ester hydrolase is present from about 30 wt % to about 40 wt %. In certain embodiments, the carboxylic ester hydrolase is present from about 10 wt % to about 20 wt %. In certain embodiments, the carboxylic ester hydrolase is present from about 5 wt % to about 20 wt %. In certain embodiments, the carboxylic ester hydrolase is present from about 5 wt % to about 15 wt %. In certain embodiments, the carboxylic ester hydrolase is present at about 5 wt %. In certain embodiments, the carboxylic ester hydrolase is present at about 1 wt %, about 2 wt %, about 3 wt %, about 4 wt %, about 5 wt %, about 6 wt %, about 7 wt %, about 8 wt %, about 9 wt % or about 10 wt %.

In certain embodiments, the carboxylic ester hydrolase is present from 1 wt % to 50 wt %. In certain embodiments, the carboxylic ester hydrolase is present from 1 wt % to 40 wt %. In certain embodiments, the carboxylic ester hydrolase is present from 1 wt % to 30 wt %. In certain embodiments, the carboxylic ester hydrolase is present from 1 wt % to 20 wt %. In certain embodiments, the carboxylic ester hydrolase is present from 1 wt % to 10 wt %. In certain embodiments, the carboxylic ester hydrolase is present from 2 wt % to 8 wt %. In certain embodiments, the carboxylic ester hydrolase is present from 30 wt % to 50 wt %. In certain embodiments, the carboxylic ester hydrolase is present from 40 wt % to 50 wt %. In certain embodiments, the carboxylic ester hydrolase is present from 20 wt % to 40 wt %. In certain embodiments, the carboxylic ester hydrolase is present from 30 wt % to 40 wt %. In certain embodiments, the carboxylic ester hydrolase is present from 10 wt % to 20 wt %. In certain embodiments, the carboxylic ester hydrolase is present from 5 wt % to 20 wt %. In certain embodiments, the carboxylic ester hydrolase is present from 5 wt % to 15 wt %. In certain embodiments, the carboxylic ester hydrolase is present at 5 wt %. In certain embodiments, the carboxylic ester hydrolase is present at 1 wt %, 2 wt %, 3 wt %, 4 wt %, 5 wt %, 6 wt %, 7 wt %, 8 wt %, 9 wt % or 10 wt %.

In some embodiments, the aqueous solution does not comprise an organic solvent. In some embodiments, the aqueous solution does not comprise a co-solvent. In certain such embodiments, the only solvent in the aqueous solution is water.

In some embodiments, the aqueous solution comprises one or more co-solvents. In some embodiments, the aqueous solution comprises one co-solvent. In some embodiments, the aqueous solution comprises two co-solvents. In some embodiments, the aqueous solution comprises three co-solvents. In some embodiments, the co-solvent is an aprotic polar solvent, a polar protic solvent, a non-polar solvent, an alcohol, PEG (e.g., PEG200, PEG400, and PEG6000), a nitrile, or an amine, or a combination of any of the foregoing. In some embodiments, the co-solvent is an alcohol (e.g., MeOH, EtOH, iPrOH, BuOH, tBuOH, nPrOH, and ethylene glycol). In some embodiments, the co-solvent is PEG (e.g., PEG200, PEG400, and PEG6000). In some embodiments, the co-solvent is DMSO, PEG (e.g., PEG200, PEG400, and PEG6000), MeOH, EtOH, iPrOH, BuOH, CH₃CN, acetone, tBuOH, nPrOH, ethylene glycol, trimethylamine, 2-MeTHF, heptane, cyclohexanes, glycerol, or THF, or a combination of any of the foregoing. In some embodiments, the co-solvent is iPrOH. In some embodiments, the aqueous solution does not comprise an additional organic solvent other than a co-solvent.

In certain embodiments, the co-solvent is present from about 5% v/v to about 50% v/v. In certain embodiments, the co-solvent is present from about 10% v/v to about 30% v/v. In certain embodiments, the co-solvent is present from about 30% v/v to about 50% v/v. In certain embodiments, the co-solvent is present from about 20% v/v to about 40% v/v. In certain embodiments, the co-solvent is present from about 10% v/v to about 25% v/v. In certain embodiments, the co-solvent is present from about 15% v/v to about 25% v/v. In certain embodiments, the co-solvent is present from about 18% v/v to about 22% v/v. In certain embodiments, the co-solvent is present at about 20% v/v. In certain embodiments, the co-solvent is present at about 15% v/v, about 16% v/v, about 17% v/v, about 18% v/v, about 19% v/v, about 20% v/v, about 21% v/v, about 22% v/v, about 23% v/v, about 24% v/v or about 25% v/v. In certain embodiments, the co-solvent is present from about 25% v/v to about 35% v/v. In certain embodiments, the co-solvent is present from about 30% v/v. In certain embodiments, the co-solvent is present at about 25% v/v, about 26% v/v, about 27% v/v, about 28% v/v, about 29% v/v, about 30% v/v, about 31% v/v, about 32% v/v, about 33% v/v, about 34% v/v or about 35% v/v. In certain embodiments, the co-solvent is present at about 5% v/v, about 6% v/v, about 7% v/v, about 8% v/v, about 9% v/v, about 10% v/v, about 11% v/v, about 12% v/v, about 13% v/v, or about 14% v/v.

In certain embodiments, the co-solvent is present from 5% v/v to 50% v/v. In certain embodiments, the co-solvent is present from 10% v/v to 30% v/v. In certain embodiments, the co-solvent is present from 15% v/v to 25% v/v. In certain embodiments, the co-solvent is present from 30% v/v to 50% v/v. In certain embodiments, the co-solvent is present from 20% v/v to 40% v/v. In certain embodiments, the co-solvent is present from 10% v/v to 25% v/v. In certain embodiments, the co-solvent is present from 18% v/v to 22% v/v. In certain embodiments, the co-solvent is present at 20% v/v. In certain embodiments, the co-solvent is present at 15% v/v, 16% v/v, 17% v/v, 18% v/v, 19% v/v, 20% v/v, 21% v/v, 22% v/v, 23% v/v, 24% v/v or 25% v/v. In certain embodiments, the co-solvent is present from 25% v/v to 35% v/v. In certain embodiments, the co-solvent is present from 30% v/v. In certain embodiments, the co-solvent is present at 25% v/v, 26% v/v, 27% v/v, 28% v/v, 29% v/v, 30% v/v, 31% v/v, 32% v/v, 33% v/v, 34% v/v or 35% v/v. In certain embodiments, the co-solvent is present at 5% v/v, 6% v/v, 7% v/v, 8% v/v, 9% v/v, 10% v/v, 11% v/v, 12% v/v, 13% v/v, or 14% v/v.

In certain embodiments, the aqueous solution comprises a PEG co-solvent. In certain embodiments, the PEG co-solvent is a PEG having a molecular weight from about 200 kDa to about 6,000 kDa. In certain embodiments, the PEG co-solvent is PEG200, PEG250, PEG400, PEG4000 or PEG6000, or a combination of any of the foregoing. In certain embodiments, the PEG co-solvent is PEG200 or PEG400 or a combination thereof. In some embodiments, the PEG co-solvent is PEG200. In some embodiments, the PEG co-solvent is PEG400.

In certain embodiments, the PEG co-solvent is present from about 5% v/v to about 50% v/v. In certain embodiments, the PEG co-solvent is present from about 10% v/v to about 30% v/v. In certain embodiments, the PEG co-solvent is present from about 30% v/v to about 50% v/v. In certain embodiments, the PEG co-solvent is present from about 20% v/v to about 40% v/v. In certain embodiments, the PEG co-solvent is present from about 10% v/v to about 25% v/v. In certain embodiments, the PEG co-solvent is present from about 15% v/v to about 25% v/v. In certain embodiments, the PEG co-solvent is present from about 18% v/v to about 22% v/v. In certain embodiments, the PEG co-solvent is present at about 20% v/v. In certain embodiments, the PEG co-solvent is present at about 15% v/v, about 16% v/v, about 17% v/v, about 18% v/v, about 19% v/v, about 20% v/v, about 21% v/v, about 22% v/v, about 23% v/v, about 24% v/v or about 25% v/v. In certain embodiments, the PEG co-solvent is present from about 25% v/v to about 35% v/v. In certain embodiments, the PEG co-solvent is present from about 30% v/v. In certain embodiments, the PEG co-solvent is present at about 25% v/v, about 26% v/v, about 27% v/v, about 28% v/v, about 29% v/v, about 30% v/v, about 31% v/v, about 32% v/v, about 33% v/v, about 34% v/v or about 35% v/v. In certain embodiments, the PEG co-solvent is present at about 5% v/v, about 6% v/v, about 7% v/v, about 8% v/v, about 9% v/v, about 10% v/v, about 11% v/v, about 12% v/v, about 13% v/v, or about 14% v/v.

In certain embodiments, the PEG co-solvent is present from 5% v/v to 50% v/v. In certain embodiments, the PEG co-solvent is present from 10% v/v to 30% v/v. In certain embodiments, the PEG co-solvent is present from 15% v/v to 25% v/v. In certain embodiments, the PEG co-solvent is present from 30% v/v to 50% v/v. In certain embodiments, the PEG co-solvent is present from 20% v/v to 40% v/v. In certain embodiments, the PEG co-solvent is present from 10% v/v to 25% v/v. In certain embodiments, the PEG co-solvent is present from 18% v/v to 22% v/v. In certain embodiments, the PEG co-solvent is present at 20% v/v. In certain embodiments, the PEG co-solvent is present at 15% v/v, 16% v/v, 17% v/v, 18% v/v, 19% v/v, 20% v/v, 21% v/v, 22% v/v, 23% v/v, 24% v/v or 25% v/v. In certain embodiments, the PEG co-solvent is present from 25% v/v to 35% v/v. In certain embodiments, the PEG co-solvent is present from 30% v/v. In certain embodiments, the PEG co-solvent is present at 25% v/v, 26% v/v, 27% v/v, 28% v/v, 29% v/v, 30% v/v, 31% v/v, 32% v/v, 33% v/v, 34% v/v or 35% v/v. In certain embodiments, the PEG co-solvent is present at 5% v/v, 6% v/v, 7% v/v, 8% v/v, 9% v/v, 10% v/v, 11% v/v, 12% v/v, 13% v/v, or 14% v/v.

In some embodiments, the concentration of the enantiomeric mixture of Formula I-i in the aqueous solution is about 15 mL/g. In some embodiments, the concentration of the enantiomeric mixture of Formula I-i in the aqueous solution is about 5 mL/g. In some embodiments, the concentration of the enantiomeric mixture of Formula I-i in the aqueous solution is about 10 mL/g. In some embodiments, the concentration of the enantiomeric mixture of Formula I-i in the aqueous solution is about 20 mL/g. In some embodiments, the concentration of the enantiomeric mixture of Formula I-i in the aqueous solution is about 25 mL/g. In some embodiments, the concentration of the enantiomeric mixture of Formula I-i in the aqueous solution is about 5 mL/g to about 100 mL/g. In some embodiments, the concentration of the enantiomeric mixture of Formula I-i in the aqueous solution is about 10 mL/g to about 50 mL/g. In some embodiments, the concentration of the enantiomeric mixture of Formula I-i in the aqueous solution is about 5 mL/g to about 25 mL/g. In some embodiments, the concentration of the enantiomeric mixture of Formula I-i in the aqueous solution is about 10 mL/g to about 20 mL/g.

In some embodiments, the concentration of the enantiomeric mixture of Formula I-i in the aqueous solution is 15 mL/g. In some embodiments, the concentration of the enantiomeric mixture of Formula I-i in the aqueous solution is 5 mL/g. In some embodiments, the concentration of the enantiomeric mixture of Formula I-i in the aqueous solution is 10 mL/g. In some embodiments, the concentration of the enantiomeric mixture of Formula I-i in the aqueous solution is 20 mL/g. In some embodiments, the concentration of the enantiomeric mixture of Formula I-i in the aqueous solution is 25 mL/g. In some embodiments, the concentration of the enantiomeric mixture of Formula I-i in the aqueous solution is 5 mL/g to 100 mL/g. In some embodiments, the concentration of the enantiomeric mixture of Formula I-i in the aqueous solution is 10 mL/g to 50 mL/g. In some embodiments, the concentration of the enantiomeric mixture of Formula I-i in the aqueous solution is 5 mL/g to 25 mL/g. In some embodiments, the concentration of the enantiomeric mixture of Formula I-i in the aqueous solution is 10 mL/g to 20 mL/g.

In some embodiments, the aqueous solution comprises a buffer. In some embodiments, the buffer has a buffer capacity from about pH 8.0 to about pH 11.0. In some embodiments, the buffer has a buffer capacity from pH 8.0 to pH 11.0. In certain embodiments, the buffer comprises glycine. In some embodiments, the buffer comprises Tris. In some embodiments, the buffer comprises phosphate. In some embodiments, the buffer comprises phosphate buffered saline. In some embodiments, the buffer comprises asparagine. In some embodiments, the buffer comprises PIPES (piperazine-N,N′-bis(2-ethanesulfonic acid)). In some embodiments, the buffer comprises HEPES (N-(2-hydroxyethyl) piperazine-N′-(2-ethanesulfonic acid)).

In certain embodiments, the aqueous solution has a basic pH. In certain embodiments, the aqueous solution has a pH from about 8.0 to about 11.0. In certain embodiments, the aqueous solution has a pH from about 8.5 to about 10.5. In certain embodiments, the aqueous solution has a pH from about 9.0 to about 11.0. In certain embodiments, the aqueous solution has a pH from about 8.0 to about 10.0. In certain embodiments, the aqueous solution has a pH from about 9.0 to about 10.0. In certain embodiments, the aqueous solution has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9 or about 11.0.

In certain embodiments, the aqueous solution has a pH from 8.0 to 11.0. In certain embodiments, the aqueous solution has a pH from 8.5 to 10.5. In certain embodiments, the aqueous solution has a pH from 9.0 to 11.0. In certain embodiments, the aqueous solution has a pH from 8.0 to 10.0. In certain embodiments, the aqueous solution has a pH from 9.0 to 10.0. In certain embodiments, the aqueous solution has a pH of 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9 or 11.0.

In certain embodiments, the aqueous solution further comprises a buffer. In certain embodiments, the buffer is present in an amount from about 0.05 M to about 1.5 M. In certain embodiments, the buffer is present in an amount from about 0.1 M to about 1.5 M. In certain embodiments, the buffer is present in an amount from about 0.5 M to about 1.5 M. In certain embodiments, the buffer is present in an amount from about 0.8 M to about 1.2 M. In certain embodiments, the buffer is present in an amount of about 0.1 M. In certain embodiments, the buffer is present in an amount of about 0.2 M. In certain embodiments, the buffer is present in an amount of about 1 M.

In certain embodiments, the buffer is present in an amount from 0.05 M to 1.5 M. In certain embodiments, the buffer is present in an amount from 0.1 M to 1.5 M. In certain embodiments, the buffer is present in an amount from 0.5 M to 1.5 M. In certain embodiments, the buffer is present in an amount from 0.8 M to 1.2 M. In certain embodiments, the buffer is present in an amount of 1 M. In certain embodiments, the buffer is present in an amount of 0.1 M. In certain embodiments, the buffer is present in an amount of 0.2 M.

In certain embodiments, the contacting is at a temperature from about 25° C. to about 50° C. In certain embodiments, the contacting is at a temperature from about 30° C. to about 50° C. In certain embodiments, the contacting is at a temperature from about 30° C. to about 40° C. In certain embodiments, the contacting is at a temperature from about 32° C. to about 38° C. In certain embodiments, the contacting is at a temperature from about 33° C. to about 35° C. In certain embodiments, the contacting is at a temperature from about 32° C. to about 36° C. In certain embodiments, the contacting is at a temperature from about 42° C. to about 48° C. In certain embodiments, the contacting is at a temperature of about 45° C. In certain embodiments, the contacting is at a temperature of about 33° C. In certain embodiments, the contacting is at a temperature of about 34° C. In certain embodiments, the contacting is at a temperature of about 35° C. In certain embodiments, the contacting is at a temperature of about 36° C. In certain embodiments, the contacting is at a temperature of about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

In certain embodiments, the contacting is at a temperature from 25° C. to 50° C. In certain embodiments, the contacting is at a temperature from 30° C. to 50° C. In certain embodiments, the contacting is at a temperature from 30° C. to 40° C. In certain embodiments, the contacting is at a temperature from 32° C. to 38° C. In certain embodiments, the contacting is at a temperature from 33° C. to 35° C. In certain embodiments, the contacting is at a temperature from 32° C. to 36° C. In certain embodiments, the contacting is at a temperature from 42° C. to 48° C. In certain embodiments, the contacting is at a temperature of 45° C. In certain embodiments, the contacting is at a temperature of 33° C. In certain embodiments, the contacting is at a temperature of 34° C. In certain embodiments, the contacting is at a temperature of 35° C. In certain embodiments, the contacting is at a temperature of 36° C. In certain embodiments, the contacting is at a temperature of 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In certain embodiments, the method provides a compound of Formula I, or a salt or solvate thereof, at a yield of at least 10%. In certain embodiments, the method provides a compound of Formula I, or a salt or solvate thereof, at a yield of at least 20%. In certain embodiments, the method provides a compound of Formula I, or a salt or solvate thereof, at a yield of at least 30%. In certain embodiments, the method provides a compound of Formula I, or a salt or solvate thereof, at a yield of at least 40%. In certain embodiments, the method provides a compound of Formula I, or a salt or solvate thereof, at a yield of at least 50%. In certain embodiments, the method provides a compound of Formula I, or a salt or solvate thereof, at a yield of at least 60%. In certain embodiments, the method provides a compound of Formula I, or a salt or solvate thereof, at a yield of at least 70%. In certain embodiments, the method provides a compound of Formula I, or a salt or solvate thereof, at a yield of at least 80%. In certain embodiments, the method provides a compound of Formula I, or a salt or solvate thereof, at a yield of at least 90%. In certain embodiments, the method provides a compound of Formula I, or a salt or solvate thereof, at a yield of at least 91%. In certain embodiments, the method provides a compound of Formula I, or a salt or solvate thereof, at a yield of at least 92%. In certain embodiments, the method provides a compound of Formula I, or a salt or solvate thereof, at a yield of at least 93%. In certain embodiments, the method provides a compound of Formula I, or a salt or solvate thereof, at a yield of at least 94%. In certain embodiments, the method provides a compound of Formula I, or a salt or solvate thereof, at a yield of at least 95%. In certain embodiments, the method provides a compound of Formula I, or a salt or solvate thereof, at a yield of at least 96%. In certain embodiments, the method provides a compound of Formula I, or a salt or solvate thereof, at a yield of at least 97%. In certain embodiments, the method provides a compound of Formula I, or a salt or solvate thereof, at a yield of at least 98%. In certain embodiments, the method provides a compound of Formula I, or a salt or solvate thereof, at a yield of at least 99%. In some embodiments, the yield is at least 98%, or at least 99%, or at least 99.9%.

In certain embodiments, the method provides a compound of Formula I, or a salt or solvate thereof, at a yield of about 90%. In certain embodiments, the method provides a compound of Formula I, or a salt or solvate thereof, at a yield of about 91%. In certain embodiments, the method provides a compound of Formula I, or a salt or solvate thereof, at a yield of about 92%. In certain embodiments, the method provides a compound of Formula I, or a salt or solvate thereof, at a yield of about 93%. In certain embodiments, the method provides a compound of Formula I, or a salt or solvate thereof, at a yield of about 94%. In certain embodiments, the method provides a compound of Formula I, or a salt or solvate thereof, at a yield of about 95%. In certain embodiments, the method provides a compound of Formula I, or a salt or solvate thereof, at a yield of about 96%. In certain embodiments, the method provides a compound of Formula I, or a salt or solvate thereof, at a yield of about 97%. In certain embodiments, the method provides a compound of Formula I, or a salt or solvate thereof, at a yield of about 98%. In certain embodiments, the method provides a compound of Formula I, or a salt or solvate thereof, at a yield of about 99%.

In certain embodiments, the method provides a compound of Formula I, or a salt or solvate thereof, at a yield of 90%. In certain embodiments, the method provides a compound of Formula I, or a salt or solvate thereof, at a yield of 91%. In certain embodiments, the method provides a compound of Formula I, or a salt or solvate thereof, at a yield of 92%. In certain embodiments, the method provides a compound of Formula I, or a salt or solvate thereof, at a yield of 93%. In certain embodiments, the method provides a compound of Formula I, or a salt or solvate thereof, at a yield of 94%. In certain embodiments, the method provides a compound of Formula I, or a salt or solvate thereof, at a yield of 95%. In certain embodiments, the method provides a compound of Formula I, or a salt or solvate thereof, at a yield of 96%. In certain embodiments, the method provides a compound of Formula I, or a salt or solvate thereof, at a yield of 97%. In certain embodiments, the method provides a compound of Formula I, or a salt or solvate thereof, at a yield of 98%. In certain embodiments, the method provides a compound of Formula I, or a salt or solvate thereof, at a yield of 99%.

In certain embodiments, the compound of Formula I, or a salt or solvate thereof:

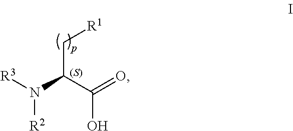

or a salt or solvate thereof, has an enantiomeric excess of at least 80%. In certain embodiments, the compound of Formula I, or a salt or solvate thereof, has an enantiomeric excess of at least 85%. In certain embodiments, the compound of Formula I, or a salt or solvate thereof, has an enantiomeric excess of at least 90%. In certain embodiments, the compound of Formula I, or a salt or solvate thereof, has an enantiomeric excess of at least 91%. In certain embodiments, the compound of Formula I, or a salt or solvate thereof, has an enantiomeric excess of at least 92%. In certain embodiments, the compound of Formula I, or a salt or solvate thereof, has an enantiomeric excess of at least 93%. In certain embodiments, the compound of Formula I, or a salt or solvate thereof, has an enantiomeric excess of at least 94%. In certain embodiments, the compound of Formula I, or a salt or solvate thereof, has an enantiomeric excess of at least 95%. In certain embodiments, the compound of Formula I, or a salt or solvate thereof, has an enantiomeric excess of at least 96%. In certain embodiments, the compound of Formula I, or a salt or solvate thereof, has an enantiomeric excess of at least 97%. In certain embodiments, the compound of Formula I, or a salt or solvate thereof, has an enantiomeric excess of at least 98%. In certain embodiments, the compound of Formula I, or a salt or solvate thereof, has an enantiomeric excess of at least 99%. In some embodiments, the enantiomeric excess is at least 98%, or at least 99%.

In certain embodiments, the compound of Formula I, or a salt or solvate thereof, has an enantiomeric excess of about 90%. In certain embodiments, the compound of Formula I, or a salt or solvate thereof, has an enantiomeric excess of about 91%. In certain embodiments, the compound of Formula I, or a salt or solvate thereof, has an enantiomeric excess of about 92%. In certain embodiments, the compound of Formula I, or a salt or solvate thereof, has an enantiomeric excess of about 93%. In certain embodiments, the compound of Formula I, or a salt or solvate thereof, has an enantiomeric excess of about 94%. In certain embodiments, the compound of Formula I, or a salt or solvate thereof, has an enantiomeric excess of about 95%. In certain embodiments, the compound of Formula I, or a salt or solvate thereof, has an enantiomeric excess of about 96%. In certain embodiments, the compound of Formula I, or a salt or solvate thereof, has an enantiomeric excess of about 97%. In certain embodiments, the compound of Formula I, or a salt or solvate thereof, has an enantiomeric excess of about 98%. In certain embodiments, the compound of Formula I, or a salt or solvate thereof, has an enantiomeric excess of about 99%. In certain embodiments, the compound of Formula I, or a salt or solvate thereof, has an enantiomeric excess of 90%. In certain embodiments, the compound of Formula I, or a salt or solvate thereof, has an enantiomeric excess of 91%. In certain embodiments, the compound of Formula I, or a salt or solvate thereof, has an enantiomeric excess of 92%. In certain embodiments, the compound of Formula I, or a salt or solvate thereof, has an enantiomeric excess of 93%. In certain embodiments, the compound of Formula I, or a salt or solvate thereof, has an enantiomeric excess of 94%. In certain embodiments, the compound of Formula I, or a salt or solvate thereof, has an enantiomeric excess of 95%. In certain embodiments, the compound of Formula I, or a salt or solvate thereof, has an enantiomeric excess of 96%. In certain embodiments, the compound of Formula I, or a salt or solvate thereof, has an enantiomeric excess of 97%. In certain embodiments, the compound of Formula I, or a salt or solvate thereof, has an enantiomeric excess of 98%. In certain embodiments, the compound of Formula I, or a salt or solvate thereof, has an enantiomeric excess of 99%.

In certain embodiments, the method of the disclosure does not comprise chromatographic purification. In some embodiments, the method of the disclosure comprises one chromatographic purification.

In certain embodiments, the enantiomeric mixture of Formula I-i is racemic.

In certain embodiments, the contacting is for a period of from about 1 hour to about 72 hours. In certain embodiments, the contacting is for a period of from about 8 hours to about 48 hours. In certain embodiments, the contacting is for a period of from about 48 hours to about 72 hours. In certain embodiments, the contacting is for a period of from about 36 hours to about 48 hours. In certain embodiments, the contacting is for a period of from about 36 hours to about 60 hours. In certain embodiments, the contacting is for a period of from about 36 hours to about 72 hours. In certain embodiments, the contacting is for a period of from about 60 hours to about 72 hours. In certain embodiments, the contacting is for a period of from about 12 hours to about 36 hours. In certain embodiments, the contacting is for a period of from about 12 hours to about 48 hours. In certain embodiments, the contacting is for a period of from about 24 hours to about 36 hours. In certain embodiments, the contacting is for a period of from about 24 hours to about 48 hours. In certain embodiments, the contacting is for a period of from about 24 hours to about 60 hours. In certain embodiments, the contacting is for a period of from about 16 hours to about 24 hours. In certain embodiments, the contacting is for a period of from about 8 hours to about 24 hours. In certain embodiments, the contacting is for a period of from about 8 hours to about 36 hours. In certain embodiments, the contacting is for about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, or about 50 hours.

In certain embodiments, the contacting is for a period of from 1 hour to 72 hours. In certain embodiments, the contacting is for a period of from 8 hours to 48 hours. In certain embodiments, the contacting is for a period of from 48 hours to 72 hours. In certain embodiments, the contacting is for a period of from 36 hours to 48 hours. In certain embodiments, the contacting is for a period of from 36 hours to 60 hours. In certain embodiments, the contacting is for a period of from 36 hours to 72 hours. In certain embodiments, the contacting is for a period of from 60 hours to 72 hours. In certain embodiments, the contacting is for a period of from 12 hours to 36 hours. In certain embodiments, the contacting is for a period of from 12 hours to 48 hours. In certain embodiments, the contacting is for a period of from 24 hours to 36 hours. In certain embodiments, the contacting is for a period of from 24 hours to 48 hours. In certain embodiments, the contacting is for a period of from 24 hours to 60 hours. In certain embodiments, the contacting is for a period of from 16 hours to 24 hours. In certain embodiments, the contacting is for a period of from 8 hours to 24 hours. In certain embodiments, the contacting is for a period of from 8 hours to 36 hours. In certain embodiments, the contacting is for 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, or 50 hours.

In certain embodiments, the enantiomeric mixture of Formula I-i and the corresponding compound of Formula I, or a salt or solvate thereof, are:

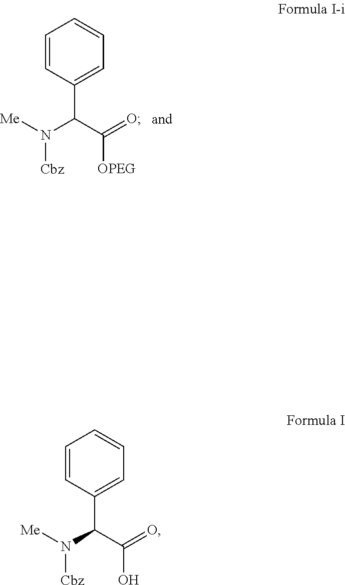

or a salt or solvate thereof; or,

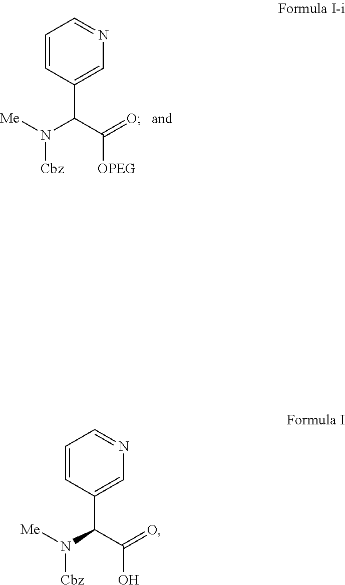

or a salt or solvate thereof; or,

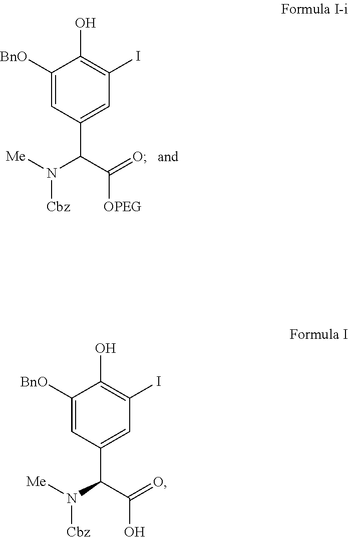

or a salt or solvate thereof; or,

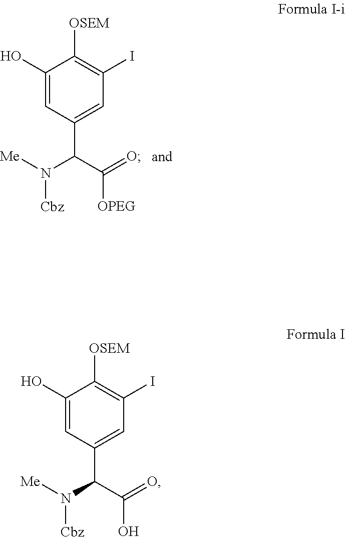

or a salt or solvate thereof; or,

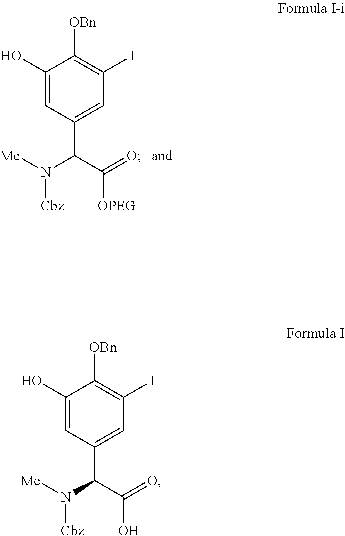

or a salt or solvate thereof; or,

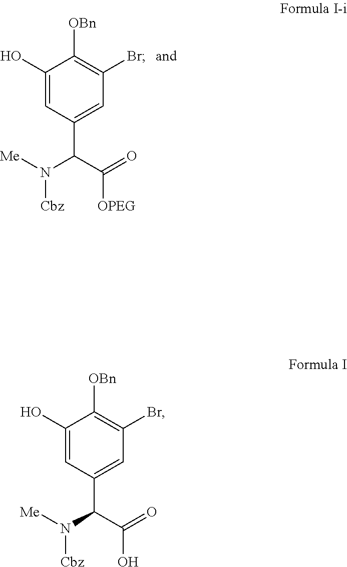

or a salt or solvate thereof; or,

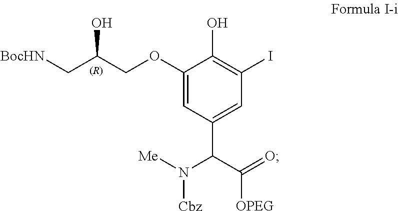

### and Formula I is

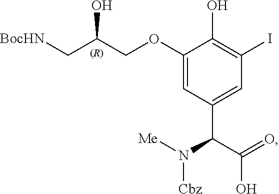

or a salt or solvate thereof; or,

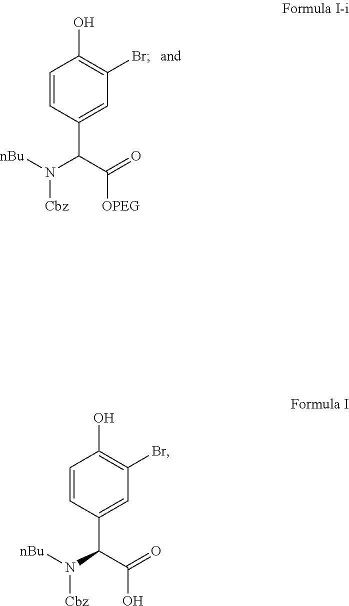

or a salt or solvate thereof; or, Formula I-i is

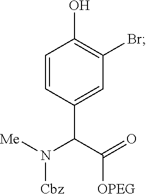

### and Formula I is

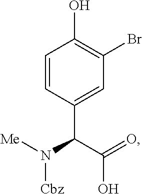 [SMILES: CN(C(=O)OCc1ccccc1)[C@H](C(=O)O)c1ccc(O)c(Br)c1]

or a salt or solvate thereof; or,

### Formula I-i is

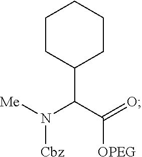

### and Formula I is

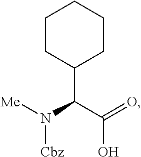 [SMILES: CN(C(=O)OCc1ccccc1)[C@H](C(=O)O)C1CCCCC1]

or a salt or solvate thereof; or,

### Formula I-i is

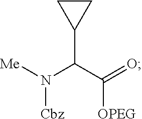

### and Formula I is

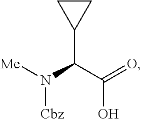 [SMILES: CN(C(=O)OCc1ccccc1)[C@H](C(=O)O)C1CC1]

or a salt or solvate thereof; or,

### Formula I-i is

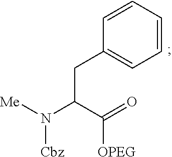

### and Formula I is

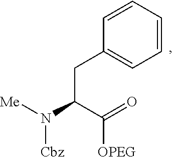

or a salt or solvate thereof; or,

### Formula I-i is

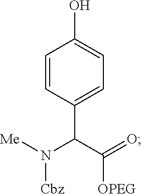

### and Formula I is

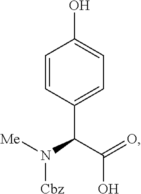 [SMILES: CN(C(=O)OCc1ccccc1)[C@H](C(=O)O)c1ccc(O)cc1]

or a salt or solvate thereof; or,

### Formula I-i is

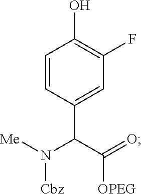

### and Formula I is

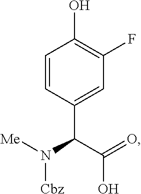 [SMILES: CN(C(=O)OCc1ccccc1)[C@H](C(=O)O)c1ccc(O)c(F)c1]

or a salt or solvate thereof; or,

### Formula I-i is

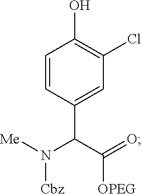

### and Formula I is

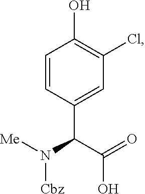 [SMILES: CN(C(=O)OCc1ccccc1)[C@H](C(=O)O)c1ccc(O)c(Cl)c1]

or a salt or solvate thereof; or,

### Formula I-i is

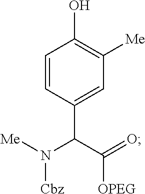

### and Formula I is

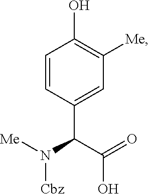 [SMILES: Cc1cc([C@@H](C(=O)O)N(C)C(=O)OCc2ccccc2)ccc1O]

or a salt or solvate thereof; or,

### Formula I-i is

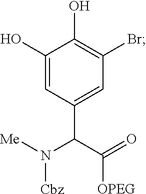

### and Formula I is

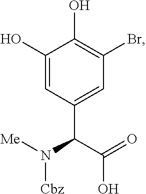 [SMILES: CN(C(=O)OCc1ccccc1)[C@H](C(=O)O)c1cc(O)c(O)c(Br)c1]

or a salt or solvate thereof; or,

### Formula I-i is

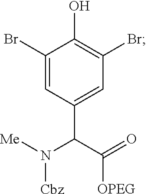

### and Formula I is

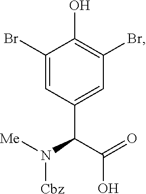 [SMILES: CN(C(=O)OCc1ccccc1)[C@H](C(=O)O)c1cc(Br)c(O)c(Br)c1]

or a salt or solvate thereof; or,

### Formula I-i is

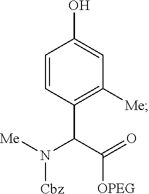

### and Formula I is

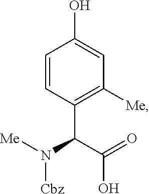 [SMILES: Cc1cc(O)ccc1[C@@H](C(=O)O)N(C)C(=O)OCc1ccccc1]

or a salt or solvate thereof; or,

### Formula I-i is

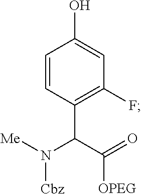

### and Formula I is

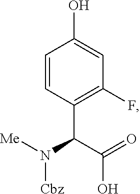 [SMILES: CN(C(=O)OCc1ccccc1)[C@H](C(=O)O)c1ccc(O)cc1F]

or a salt or solvate thereof; or,

### Formula I-i is

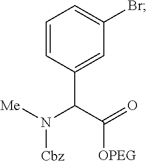

### and Formula I is

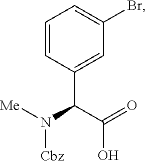 [SMILES: CN(C(=O)OCc1ccccc1)[C@H](C(=O)O)c1cccc(Br)c1]

or a salt or solvate thereof; or,

### Formula I-i is

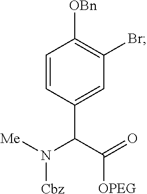

### and Formula I is

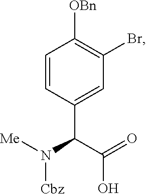 [SMILES: CN(C(=O)OCc1ccccc1)[C@H](C(=O)O)c1ccc(OCc2ccccc2)c(Br)c1]

or a salt or solvate thereof; or,

### Formula I-i is

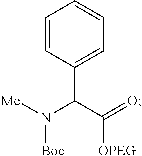

### and Formula I is

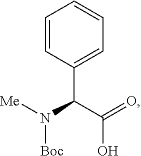 [SMILES: CN(C(=O)OC(C)(C)C)[C@H](C(=O)O)c1ccccc1]

or a salt or solvate thereof;
wherein, in each instance, PEG is —(CH₂CH₂—O)_{q}—CH₃, wherein q is 1, 2, or 3. In some embodiments, q is 1. In some embodiments, q is 2. In some embodiments, q is 3.

In some embodiments, the enantiomeric mixture of Formula I-i and the corresponding compound of Formula I, or a salt or solvate thereof, are:

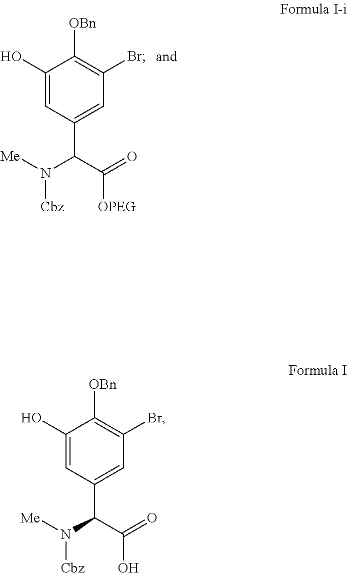

or a salt or solvate thereof;
wherein PEG is —(CH₂CH₂—O)_{q}—CH₃, wherein q is 1, 2, or 3. In some embodiments, q is 1. In some embodiments, q is 2. In some embodiments, q is 3.

In some embodiments, the enantiomeric mixture of Formula I-i and the corresponding compound of Formula I, or a salt or solvate thereof, are:

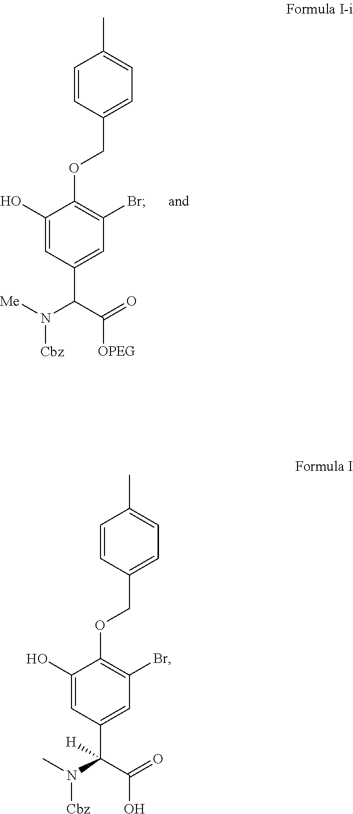

or a salt or solvate thereof;
wherein PEG is —(CH₂CH₂—O)_{q}—CH₃, wherein q is 1, 2, or 3. In some embodiments, q is 1. In some embodiments, q is 2. In some embodiments, q is 3.

In some embodiments, the enantiomeric mixture of Formula I-i and the corresponding compound of Formula I, or a salt or solvate thereof, are:

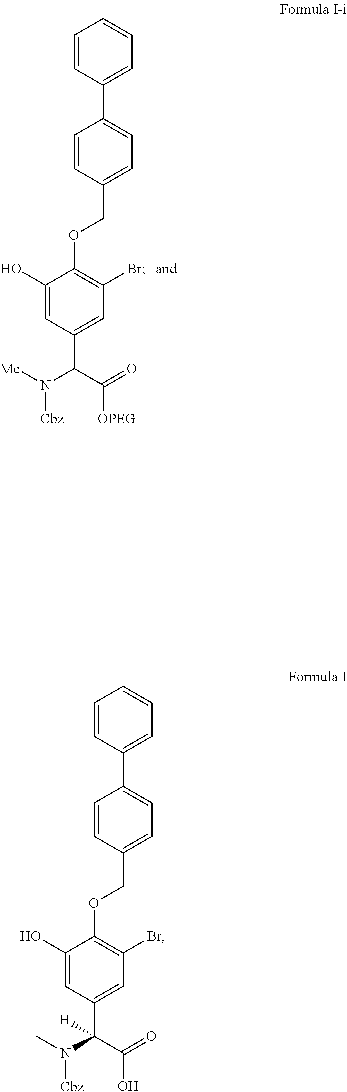

or a salt or solvate thereof;
wherein PEG is —(CH₂CH₂—O)_{q}—CH₃, wherein q is 1, 2, or 3. In some embodiments, q is 1. In some embodiments, q is 2. In some embodiments, q is 3.

In certain embodiments, the enantiomeric mixture of Formula I-i and the corresponding compound of Formula I, or a salt or solvate thereof, are:

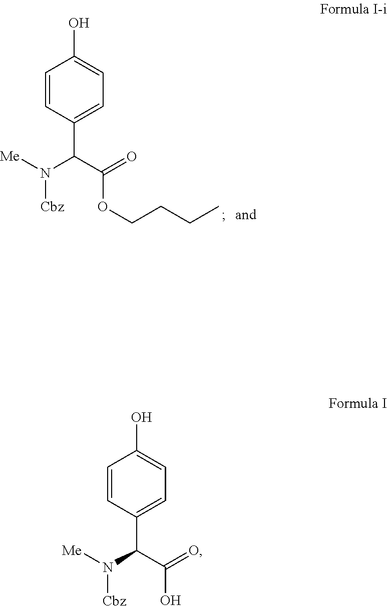 [SMILES: CCCCOC(=O)C(c1ccc(O)cc1)N(C)C(=O)OCc1ccccc1 ; CN(C(=O)OCc1ccccc1)[C@H](C(=O)O)c1ccc(O)cc1]

or a salt or solvate thereof; or,

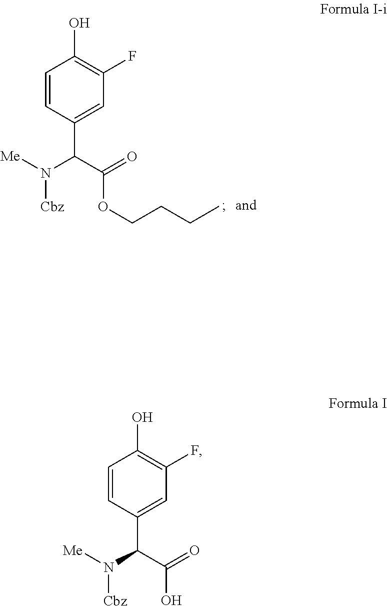 [SMILES: CCCCOC(=O)C(c1ccc(O)c(F)c1)N(C)C(=O)OCc1ccccc1 ; CN(C(=O)OCc1ccccc1)[C@H](C(=O)O)c1ccc(O)c(F)c1]

or a salt or solvate thereof; or,

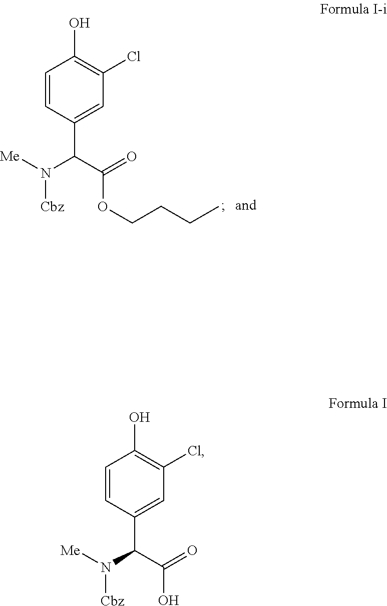 [SMILES: CCCCOC(=O)C(c1ccc(O)c(Cl)c1)N(C)C(=O)OCc1ccccc1 ; CN(C(=O)OCc1ccccc1)[C@H](C(=O)O)c1ccc(O)c(Cl)c1]

or a salt or solvate thereof; or,

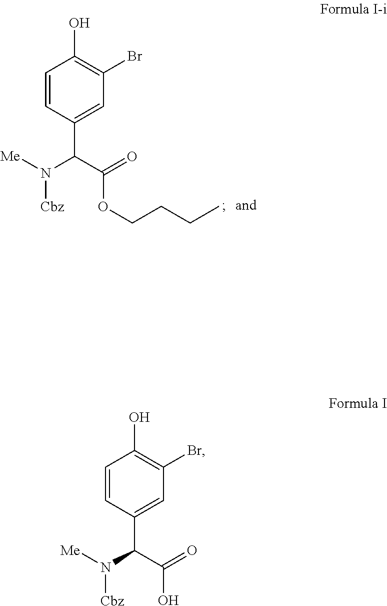 [SMILES: CCCCOC(=O)C(c1ccc(O)c(Br)c1)N(C)C(=O)OCc1ccccc1 ; CN(C(=O)OCc1ccccc1)[C@H](C(=O)O)c1ccc(O)c(Br)c1]

or a salt or solvate thereof; or,

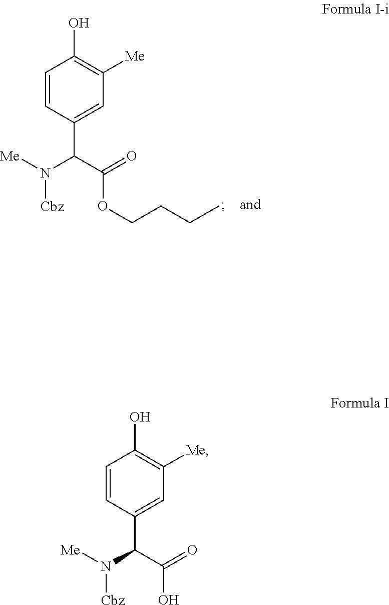 [SMILES: CCCCOC(=O)C(c1ccc(O)c(C)c1)N(C)C(=O)OCc1ccccc1 ; Cc1cc([C@@H](C(=O)O)N(C)C(=O)OCc2ccccc2)ccc1O]

or a salt or solvate thereof; or,

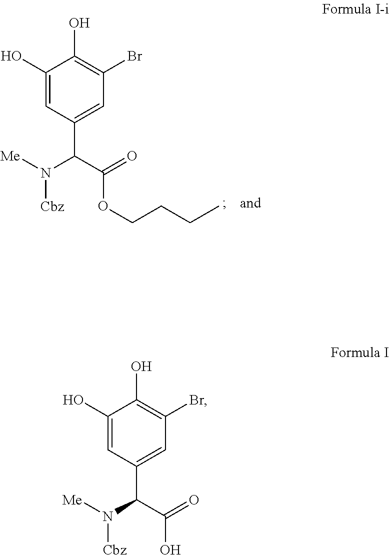 [SMILES: CCCCOC(=O)C(c1cc(O)c(O)c(Br)c1)N(C)C(=O)OCc1ccccc1 ; CN(C(=O)OCc1ccccc1)[C@H](C(=O)O)c1cc(O)c(O)c(Br)c1]

or a salt or solvate thereof; or,

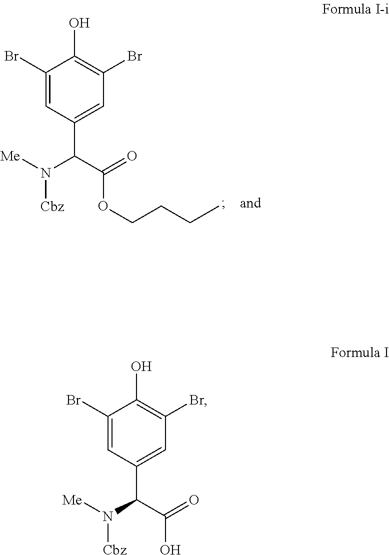 [SMILES: CCCCOC(=O)C(c1cc(Br)c(O)c(Br)c1)N(C)C(=O)OCc1ccccc1 ; CN(C(=O)OCc1ccccc1)[C@H](C(=O)O)c1cc(Br)c(O)c(Br)c1]

or a salt or solvate thereof; or,

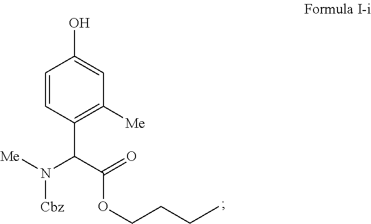 [SMILES: CCCCOC(=O)C(c1ccc(O)cc1C)N(C)C(=O)OCc1ccccc1]

### and Formula I is

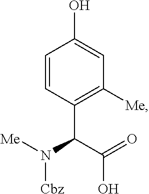 [SMILES: Cc1cc(O)ccc1[C@@H](C(=O)O)N(C)C(=O)OCc1ccccc1]

or a salt or solvate thereof; or,

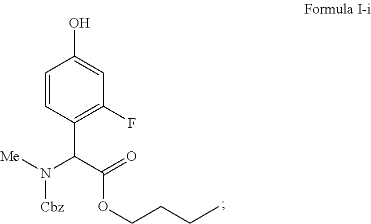 [SMILES: CCCCOC(=O)C(c1ccc(O)cc1F)N(C)C(=O)OCc1ccccc1]

and Formula I is

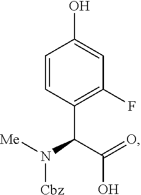 [SMILES: CN(C(=O)OCc1ccccc1)[C@H](C(=O)O)c1ccc(O)cc1F]

or a salt or solvate thereof; or,

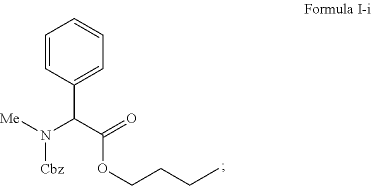 [SMILES: CCCCOC(=O)C(c1ccccc1)N(C)C(=O)OCc1ccccc1]

### and Formula I is

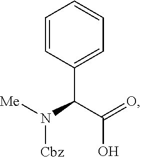 [SMILES: CN(C(=O)OCc1ccccc1)[C@H](C(=O)O)c1ccccc1]

or a salt or solvate thereof; or,

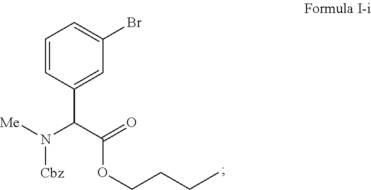 [SMILES: CCCCOC(=O)C(c1cccc(Br)c1)N(C)C(=O)OCc1ccccc1]

and Formula I is

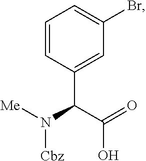 [SMILES: CN(C(=O)OCc1ccccc1)[C@H](C(=O)O)c1cccc(Br)c1]

or a salt or solvate thereof; or,

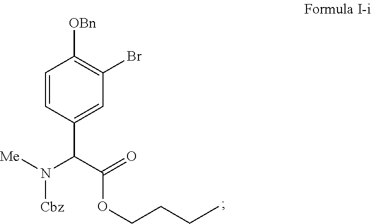 [SMILES: CCCCOC(=O)C(c1ccc(OCc2ccccc2)c(Br)c1)N(C)C(=O)OCc1ccccc1]

### and Formula I is

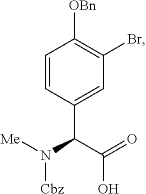 [SMILES: CN(C(=O)OCc1ccccc1)[C@H](C(=O)O)c1ccc(OCc2ccccc2)c(Br)c1]

or a salt or solvate thereof.

In some embodiments, the subject matter described herein is directed to a method of preparing an enantiomerically enriched compound of Formula I:

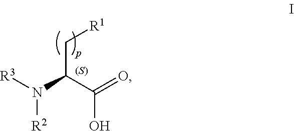

or a salt or solvate thereof,
wherein,
   R¹ is selected from the group consisting of: C₃₋₆ cycloalkyl and phenyl, wherein each can be unsubstituted or substituted with one, two, three, or four substituents independently selected from the group consisting of hydroxyl, fluoro, chloro, bromo, and methyl;
   R² is a carbamate N-protecting group;
   R³ is methyl; and
   p is 1 or zero;
the method comprising:
contacting in an aqueous solution, wherein the aqueous solution comprises PEG400, at a temperature of about 45° C., an enantiomeric mixture of Formula I-i:

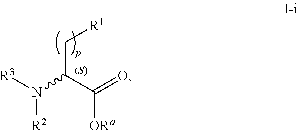

wherein,
   R¹, R², R³, and p are as defined in the compound of Formula I; and
   R^{a} is n-butyl or —(CH₂CH₂O)_{q}—CH₃, wherein q is 2 or 3;
with a solution of Lipase 147, Evoxx Technologies GmbH, evo-1.3.147.S dissolved in aqueous glycine buffer at a pH from about 9.0 to about 10.0, to produce the enantiomerically enriched compound of Formula I, or a salt or solvate thereof. In certain such embodiments, the pH is from 9.0 to 10.0. In some embodiments, R^{a} is —(CH₂CH₂O)_{q}—CH₃. In some embodiments, q is 2. In some embodiments, q is 3. In some embodiments, R^{a} is n-butyl. In some embodiments, R² is Cbz. In some embodiments, R¹ is unsubstituted or substituted C₃₋₆ cycloalkyl. In some embodiments, R¹ is unsubstituted or substituted phenyl.

In certain embodiments, the contacting is under dynamic kinetic resolution conditions.

In certain embodiments, the method comprises a work-up at a point when the reaction has run to its intended extent. In certain such embodiments, the method further comprises: a) adding a first organic solvent to an aqueous solution comprising the produced enantiomerically enriched compound of Formula I to form a first mixture; b) separating the first mixture into a first aqueous phase and a first organic phase; c) adjusting the pH of the first aqueous phase to protonate the enantiomerically enriched compound of Formula I; d) adding a second organic solvent to the first aqueous phase to form a second mixture; and e) separating the second mixture into a second aqueous phase and second organic phase, wherein the second organic phase comprises the enantiomerically enriched compound of Formula I, to prepare the enantiomerically enriched compound of Formula I, or a salt or solvate thereof.

In some embodiments wherein the method further includes a work-up, the method comprises: a) adding a first organic solvent to an aqueous solution comprising the produced enantiomerically enriched compound of Formula I to form a first mixture; b) separating the first mixture into a first aqueous phase and a first organic phase; c) adjusting the pH of the first aqueous phase to about 7.0; d) adding a second organic solvent to the first aqueous phase to form a second mixture; e) adjusting the pH of the second mixture to about 2.0 to about 3.0; f) separating the second mixture into a second aqueous phase and second organic phase; and g) concentrating the second organic phase, to prepare the enantiomerically enriched compound of Formula I, or a salt or solvate thereof. In some embodiments, the pH of the second mixture of step e) is adjusted to 2.0 to 3.0. In some embodiments, the pH of the second mixture of step e) is adjusted to about 2.5. In some embodiments, the pH of the second mixture of step e) is adjusted to 2.5. In some embodiments, the pH of the first aqueous phase of step c) is adjusted to 7.0. In some embodiments, the second mixture of step e) is filtered before the separating of step f).

In some embodiments, wherein the method includes a work-up, the method further comprises: a) adding a first organic solvent to an aqueous solution comprising the produced enantiomerically enriched compound of Formula I to form a first mixture; b) separating the first mixture into a first aqueous phase and a first organic phase; c) adding a second organic solvent to the first aqueous phase to form a second mixture; d) adjusting the pH of the second mixture to below about 7.0; e) separating the second mixture into a second aqueous phase and second organic phase; and f) concentrating the second organic phase, to prepare the enantiomerically enriched compound of Formula I, or a salt or solvate thereof. In certain such embodiments, the pH of the second mixture of step d) is adjusted to between about 2.0 to about 6.5. In some embodiments, the pH of the second mixture of step d) is adjusted to between about 2.0 to about 6.0. In some embodiments, the pH of the second mixture of step d) is adjusted to between about 2.0 to about 5.5. In some embodiments, the pH of the second mixture of step d) is adjusted to between about 2.0 to about 5.0. In some embodiments, the pH of the second mixture of step d) is adjusted to between about 2.0 to about 4.5. In some embodiments, the pH of the second mixture of step d) is adjusted to between about 2.0 to about 4.0. In some embodiments, the pH of the second mixture of step d) is adjusted to between about 2.0 to about 3.5. In some embodiments, the pH of the second mixture of step d) is adjusted to between about 2.0 to about 3.0. In some embodiments, the pH of the second mixture of step d) is adjusted to between about 2.0 to about 2.5. In some embodiments, the pH of the second mixture of step d) is adjusted to between about 3.0 to about 6.5. In some embodiments, the pH of the second mixture of step d) is adjusted to between about 3.0 to about 6.0. In some embodiments, the pH of the second mixture of step d) is adjusted to between about 3.0 to about 5.5. In some embodiments, the pH of the second mixture of step d) is adjusted to between about 3.0 to about 5.0. In some embodiments, the pH of the second mixture of step d) is adjusted to about 2.0, about 2.5, about 3.0, about 3.5, about 4.0, about 4.5, about 5.0, about 5.5, about 6.0, or about 6.5. In some embodiments, the pH of the second mixture of step d) is adjusted to between 2.0 to 6.5. In some embodiments, the pH of the second mixture of step d) is adjusted to between 2.0 to 6.0. In some embodiments, the pH of the second mixture of step d) is adjusted to between 2.0 to 5.5. In some embodiments, the pH of the second mixture of step d) is adjusted to between 2.0 to 5.0. In some embodiments, the pH of the second mixture of step d) is adjusted to between 2.0 to 4.5. In some embodiments, the pH of the second mixture of step d) is adjusted to between 2.0 to 4.0. In some embodiments, the pH of the second mixture of step d) is adjusted to between 2.0 to 3.5. In some embodiments, the pH of the second mixture of step d) is adjusted to between 2.0 to 3.0. In some embodiments, the pH of the second mixture of step d) is adjusted to between 2.0 to 2.5. In some embodiments, the pH of the second mixture of step d) is adjusted to between 3.0 to 6.5. In some embodiments, the pH of the second mixture of step d) is adjusted to between 3.0 to 6.0. In some embodiments, the pH of the second mixture of step d) is adjusted to between 3.0 to 5.5. In some embodiments, the pH of the second mixture of step d) is adjusted to between 3.0 to 5.0. In some embodiments, the pH of the second mixture of step d) is adjusted to 2.0, 2.5, 3.0, 3.5, 4.0, 4.5, 5.0, 5.5, 6.0, or 6.5. In some embodiments, the second organic phase of step e) is filtered before the concentrating of step f).

In some embodiments, wherein the method includes a work-up, the first organic solvent is isopropyl acetate, CPME, 2-MeTHF, CH₂Cl₂, MTBE, cyclohexanes, or toluene, or a combination of any of the foregoing. In some embodiments, the first organic solvent is isopropyl acetate, cyclohexanes, or MTBE, or a combination of any of the foregoing. In some embodiments, the first organic solvent is cyclohexanes or MTBE, or a combination thereof. In some embodiments, the first organic solvent is isopropyl acetate. In some embodiments, the second organic solvent is isopropyl acetate, CPME, 2-MeTHF, CH₂Cl₂, MTBE, cyclohexanes, or toluene, or a combination of any of the foregoing. In some embodiments, the second organic solvent is isopropyl acetate, cyclohexanes, or MTBE, or a combination of any of the foregoing. In some embodiments, the second organic solvent is cyclohexanes or MTBE, or a combination thereof. In some embodiments, the second organic solvent is isopropyl acetate.

In some embodiments, step a) of the work-up further comprises adding water to the aqueous solution.

In some embodiments, the method comprises isolating the enantiomerically enriched compound of Formula I, or a salt or solvate thereof.

### III. Salt Forms of the Enantiomerically Enriched Compounds of Formula I

The enantiomerically enriched compounds of Formula I of the disclosure may exist as a salt. Salts include, for example, those derived from organic bases (such as an amine (e.g., a primary, secondary or tertiary amine)), an alkali metal hydroxide, alkaline earth metal hydroxide, or the like. In some embodiments, the salt of the enantiomerically enriched compound of Formula I is a lysine, piperazine, or dicyclohexylamine salt. In some embodiments, the salt of the enantiomerically enriched compound of Formula I is a lysine salt. In some embodiments, the salt of the enantiomerically enriched compound of Formula I is a piperazine salt. In some embodiments, the salt of the enantiomerically enriched compound of Formula I is a dicyclohexylamine salt.

The disclosure provides for solid forms of the salts of the enantiomerically enriched compounds of Formula I of the disclosure. In one embodiment, a lysine salt of the enantiomerically enriched compound of Formula I is a solid form. In another embodiment, a piperazine salt of the enantiomerically enriched compound of Formula I is a solid form. In another embodiment, a dicyclohexylamine salt of the enantiomerically enriched compound of Formula I is a solid form.

The solid forms of the salts of the enantiomerically enriched compounds of Formula I of the disclosure may be crystalline forms. In one embodiment, a lysine salt of the enantiomerically enriched compound of Formula I is crystalline. In another embodiment, a piperazine salt of the enantiomerically enriched compound of Formula I is crystalline. In another embodiment, a dicyclohexylamine salt of the enantiomerically enriched compound of Formula I is crystalline.

The solid forms of the salts of the enantiomerically enriched compounds of Formula I of the disclosure may be amorphous solids. In one embodiment, a lysine salt of the enantiomerically enriched compound of Formula I is amorphous. In another embodiment, a piperazine salt of the enantiomerically enriched compound of Formula I is amorphous. In another embodiment, a dicyclohexylamine salt of the enantiomerically enriched compound of Formula I is amorphous.

The solid forms described herein, including salt forms, crystalline forms, and amorphous solids can be characterized by a number of methods including, for example, single crystal X-ray diffraction, X-ray powder diffraction (XRPD), microscopy (e.g., scanning electron microscopy (SEM)), thermal analysis (e.g., differential scanning calorimetry (DSC), dynamic vapor sorption (DVS), thermal gravimetric analysis (TGA), and hot-stage microscopy), spectroscopy (e.g., infrared, Raman, and solid-state nuclear magnetic resonance), ultra-high performance liquid chromatography (UHPLC), and proton nuclear magnetic resonance (¹H NMR).

The purity of the solid forms provided herein can be determined by standard analytical methods, such as thin layer chromatography (TLC), gel electrophoresis, gas chromatography, ultra-high performance liquid chromatography (UHPLC), chiral HPLC, and mass spectrometry (MS).

The present disclosure provides salt forms of an enantiomerically enriched compound of Formula I, wherein the enantiomerically enriched compound of Formula I is compound 2ac:

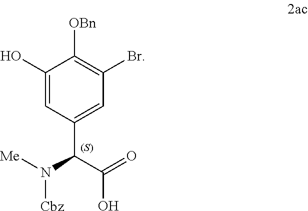

In some embodiments, the salt of compound 2ac is a lysine, piperazine, or dicyclohexylamine salt. In some embodiments, the salt of compound 2ac is a lysine salt. In some embodiments, the salt of compound 2ac is a piperazine salt. In some embodiments, the salt of compound 2ac is a dicyclohexylamine salt.

The salts of compound 2ac may exist as solid forms. In some embodiments, a lysine, piperazine, or dicyclohexylamine salt of compound 2ac is a solid form. In some embodiments, a lysine salt of compound 2ac is a solid form. In some embodiments, a piperazine salt of compound 2ac is a solid form. In some embodiments, a dicyclohexylamine salt of compound 2ac is a solid form.

The solid forms of the salts of compound 2ac may be crystalline forms. In one embodiment, a lysine salt of compound 2ac is crystalline. In another embodiment, a piperazine salt of compound 2ac is crystalline. In another embodiment, a dicyclohexylamine salt of compound 2ac is crystalline.

The solid forms of the salts of compound 2ac may be amorphous solids. In one embodiment, a lysine salt of compound 2ac is amorphous. In another embodiment, a piperazine salt of compound 2ac is amorphous. In another embodiment, a dicyclohexylamine salt of compound 2ac is amorphous.

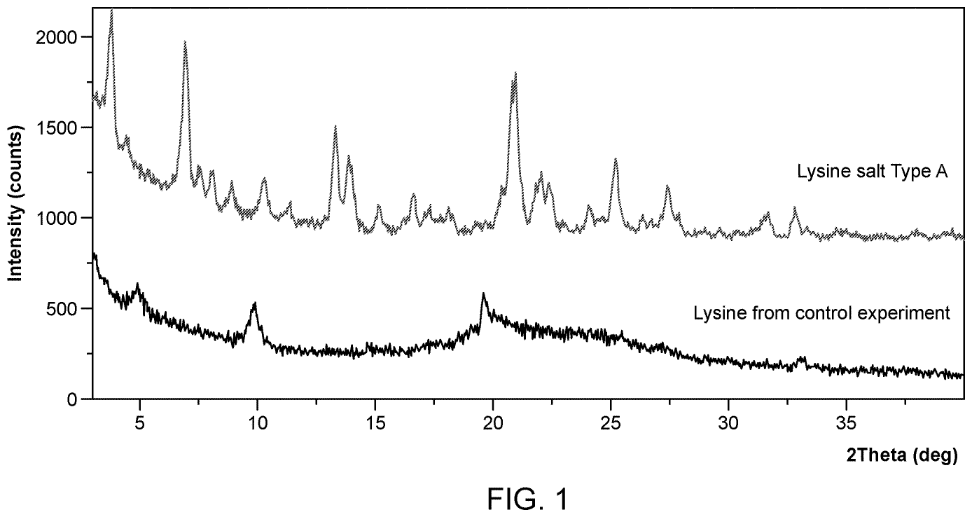

The disclosure provides a crystalline lysine salt of compound 2ac. In some embodiments, the crystalline lysine salt of compound 2ac is characterized by an XRPD pattern comprising at least one peak selected from the group consisting of 3.8, 6.9, 13.3, 13.9, 20.9, 22.0, 22.4, and 25.2 °2θ+0.1 °2θ. In some embodiments, the crystalline lysine salt of compound 2ac is characterized by an XRPD pattern comprising at least three peaks selected from the group consisting of 3.8, 6.9, 13.3, 13.9, 20.9, 22.0, 22.4, and 25.2 °2θ±0.1 °2θ. In some embodiments, the crystalline lysine salt of compound 2ac is characterized by an XRPD pattern comprising at least five peaks selected from the group consisting of 3.8, 6.9, 13.3, 13.9, 20.9, 22.0, 22.4, and 25.2 °2θ±0.1 °2θ. In some embodiments, the crystalline lysine salt of compound 2ac is characterized by an XRPD pattern having peaks at 3.8, 6.9, 13.3, 13.9, 20.9, 22.0, 22.4, and 25.2 °2θ±0.1 °2θ. In some embodiments, the crystalline lysine salt of compound 2ac is characterized by an XRPD pattern substantially the same as the pattern shown in FIG. 1. In some embodiments, the crystalline lysine salt of compound 2ac is characterized by one, two, three, four, five, six, seven, eight, nine, ten, eleven, twelve, thirteen, fourteen, fifteen, sixteen, seventeen, eighteen, nineteen, twenty, or all of the characteristic XRPD peaks as set forth in Table 7.

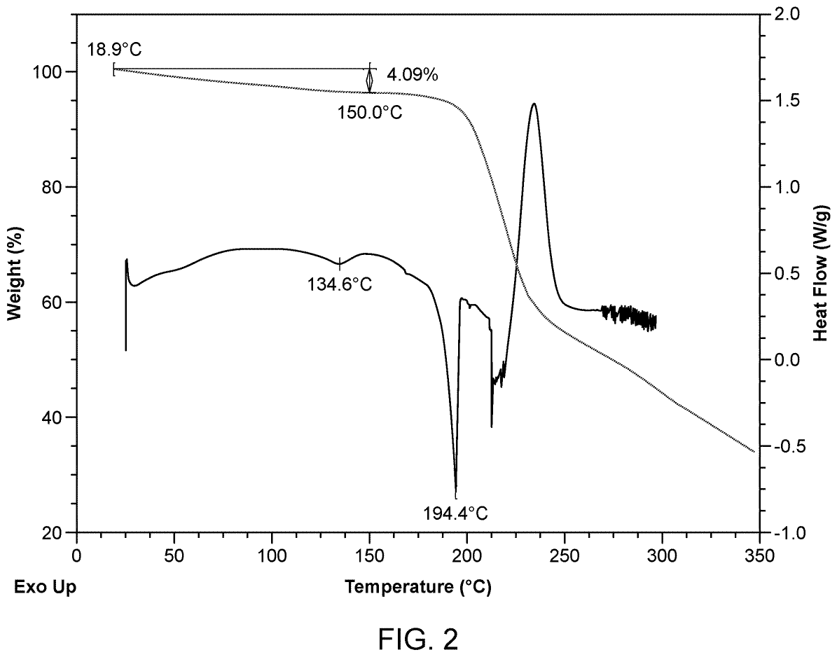

The crystalline lysine salt of compound 2ac may also be characterized by thermogravimetry (TG). In some embodiments, the crystalline lysine salt of compound 2ac is characterized by a TG thermogram indicating weight loss of about 4.09% from between about 18.9° C. to about 150° C. In some embodiments, the crystalline lysine salt of compound 2ac is characterized by a TG thermogram substantially the same as the pattern shown in FIG. 2.

The crystalline lysine salt of compound 2ac may also be characterized by differential scanning calorimetry (DSC). In some embodiments, the crystalline lysine salt of compound 2ac is characterized by a differential scanning calorimetry (DSC) thermogram comprising an endothermic peak between about 133° C. and about 137° C. In some embodiments, the crystalline lysine salt of compound 2ac is characterized by a DSC thermogram comprising an endothermic peak at about 135° C. In some embodiments, the crystalline lysine salt of compound 2ac is characterized by a DSC thermogram comprising an endothermic peak at about 134.6° C. In some embodiments, the crystalline lysine salt of compound 2ac is characterized by a differential scanning calorimetry (DSC) thermogram comprising an endothermic peak between about 192° C. and about 196° C. In some embodiments, the crystalline lysine salt of compound 2ac is characterized by a DSC thermogram comprising an endothermic peak at about 194° C. In some embodiments, the crystalline lysine salt of compound 2ac is characterized by a DSC thermogram comprising an endothermic peak at about 194.4° C. In some embodiments, the crystalline lysine salt of compound 2ac is characterized by a differential scanning calorimetry (DSC) thermogram comprising an endothermic peak between about 133° C. and about 137° C. and an endothermic peak between about 192° C. and about 196° C. In some embodiments, the crystalline lysine salt of compound 2ac is characterized by a DSC thermogram comprising an endothermic peak at about 135° C. and an endothermic peak at about 194° C. In some embodiments, the crystalline lysine salt of compound 2ac is characterized by a DSC thermogram comprising an endothermic peak at about 134.6° C. and an endothermic peak at about 194.4° C. In some embodiments, the crystalline lysine salt of compound 2ac is characterized by a DSC thermogram substantially the same as the pattern shown in FIG. 2.

In some embodiments, the crystalline lysine salt of compound 2ac is characterized by having at least two of the following:
a) an XRPD pattern comprising at least three peaks selected from the group consisting of 3.8, 6.9, 13.3, 13.9, 20.9, 22.0, 22.4, and 25.2 °2θ±0.1 °2θ;
b) an endothermic peak at about 134.6° C. and an endothermic peak at about 194.4° C. as measured by DSC; and
c) weight loss of about 4.09% from between about 18.9° C. to about 150° C. as measured by TG.

In some embodiments, the crystalline lysine salt of compound 2ac is characterized by having at least two of the following:
a) an XRPD pattern substantially the same as the pattern shown in FIG. 1;
b) a TG thermogram substantially the same as the pattern shown in FIG. 2; and
c) a DSC thermogram substantially the same as the pattern shown in FIG. 2.

The disclosure also provides methods for preparing a crystalline lysine salt of compound 2ac. In some embodiments, the method for preparing a crystalline lysine salt of compound 2ac comprises:
a) dissolving free form compound 2ac in isopropyl alcohol (IPA) to form a first mixture;
b) adding the first mixture of step a) to a suspension of L-lysine in IPA to form a second mixture;
c) stirring the second mixture of step b) at about 25° C. to obtain a first suspension;
d) adding IPA to the first suspension of step c) and stirring the resulting mixture at about 25° C. to obtain a second suspension; and
e) isolating the precipitated solids from the second suspension of step d) to afford the crystalline lysine salt of compound 2ac.

In some embodiments of the method for preparing a crystalline lysine salt of compound 2ac, the stirring of step c) occurs for about 24 hours. In some embodiments of the method for preparing a crystalline lysine salt of compound 2ac, the stirring of step d) occurs for about 24 hours.

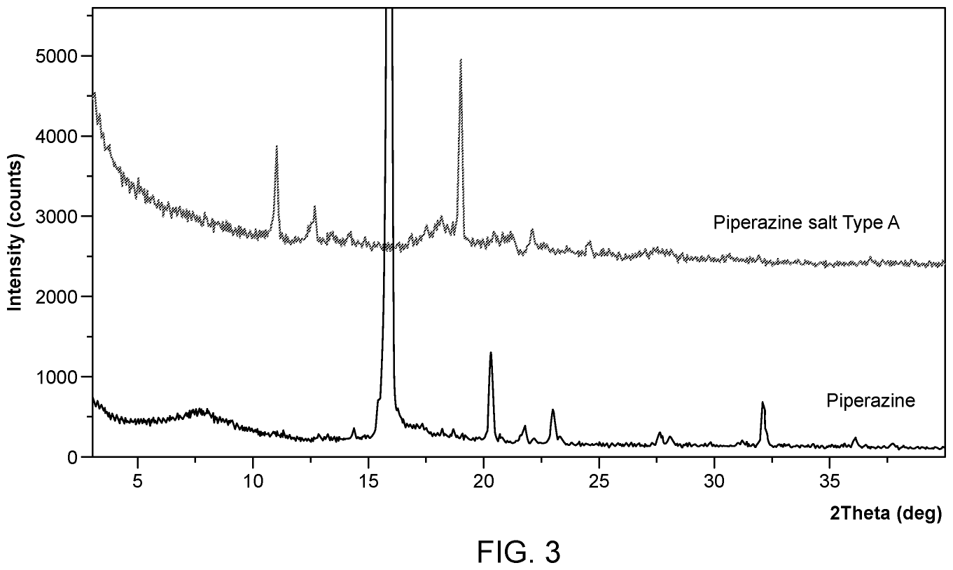

The disclosure provides a crystalline piperazine salt of compound 2ac. In some embodiments, the crystalline piperazine salt of compound 2ac is characterized by an XRPD pattern comprising at least one peak selected from the group consisting of 11.0, 12.6, and 19.0 °2θ±0.1 °2θ. In some embodiments, the crystalline piperazine salt of compound 2ac is characterized by an XRPD pattern comprising at least two peaks selected from the group consisting of 11.0, 12.6, and 19.0 °2θ±0.1 °2θ. In some embodiments, the crystalline piperazine salt of compound 2ac is characterized by an XRPD pattern having peaks at 11.0, 12.6, and 19.0 °2θ±0.1 °2θ. In some embodiments, the crystalline piperazine salt of compound 2ac is characterized by an XRPD pattern substantially the same as the pattern shown in FIG. 3. In some embodiments, the crystalline piperazine salt of compound 2ac is characterized by one, two, three, four, five, six, seven, eight, nine, ten, eleven, twelve, thirteen, fourteen, fifteen, sixteen, seventeen, eighteen, nineteen, twenty, or all of the characteristic XRPD peaks as set forth in Table 8.

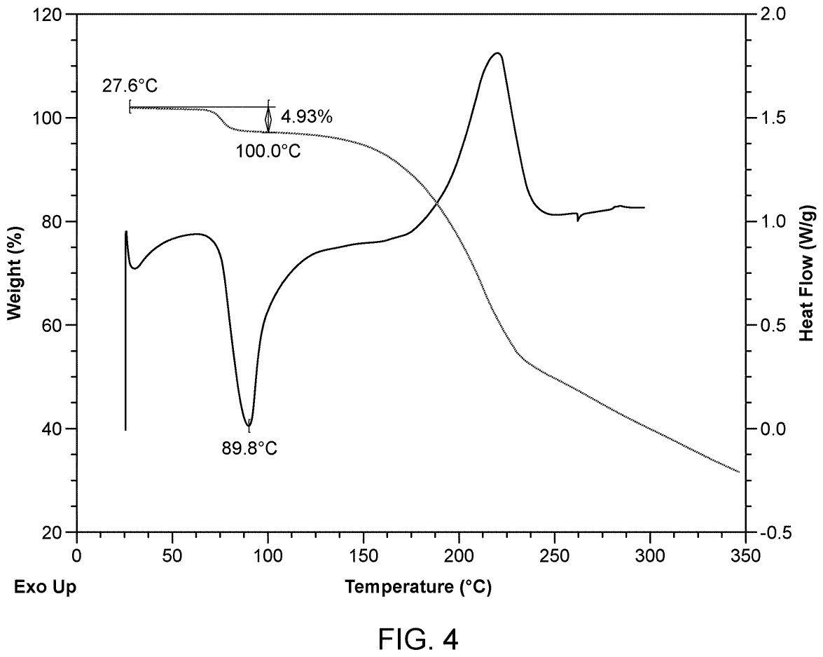

The crystalline piperazine salt of compound 2ac may also be characterized by thermogravimetry (TG). In some embodiments, the crystalline piperazine salt of compound 2ac is characterized by a TG thermogram indicating weight loss of about 4.93% from between about 27.6° C. to about 100° C. In some embodiments, the crystalline piperazine salt of compound 2ac is characterized by a TG thermogram substantially the same as the pattern shown in FIG. 4.

The crystalline piperazine salt of compound 2ac may also be characterized by differential scanning calorimetry (DSC). In some embodiments, the crystalline piperazine salt of compound 2ac is characterized by a differential scanning calorimetry (DSC) thermogram comprising an endothermic peak between about 88° C. and about 92° C. In some embodiments, the crystalline piperazine salt of compound 2ac is characterized by a DSC thermogram comprising an endothermic peak at about 90° C. In some embodiments, the crystalline piperazine salt of compound 2ac is characterized by a DSC thermogram comprising an endothermic peak at about 89.8° C. In some embodiments, the crystalline piperazine salt of compound 2ac is characterized by a DSC thermogram substantially the same as the pattern shown in FIG. 4.

In some embodiments, the crystalline piperazine salt of compound 2ac is characterized by having at least two of the following:
a) an XRPD pattern comprising at least two peaks selected from the group consisting of 11.0, 12.6, and 19.0 °2θ±0.1 °2θ;
b) an endothermic peak at about 89.8° C. as measured by DSC; and
c) weight loss of about 4.93% from between about 27.6° C. to about 100° C. as measured by TG.

In some embodiments, the crystalline piperazine salt of compound 2ac is characterized by having at least two of the following:
a) an XRPD pattern substantially the same as the pattern shown in FIG. 3;
b) a TG thermogram substantially the same as the pattern shown in FIG. 4; and
c) a DSC thermogram substantially the same as the pattern shown in FIG. 4.

The disclosure also provides methods for preparing a crystalline piperazine salt of compound 2ac. In some embodiments, the method for preparing a crystalline piperazine salt of compound 2ac comprises:
a) dissolving free form compound 2ac in ethyl acetate to form a first mixture;
b) adding to the first mixture of step a) a solution of piperazine in ethyl acetate to form a second mixture;
c) stirring the second mixture of step b) at about 25° C.;
d) after the stirring of step c), seeding the second mixture with compound 2ac piperazine salt to form a third mixture;
e) stirring the third mixture of step d) at about 25° C. to form a suspension; and
f) isolating the precipitated solids from the suspension of step e) to afford the crystalline piperazine salt of compound 2ac.

In some embodiments of the method for preparing a crystalline piperazine salt of compound 2ac, the stirring of step c) occurs for about 1.5 hours. In some embodiments of the method for preparing a crystalline piperazine salt of compound 2ac, the stirring of step e) occurs for about 2 hours.

In some embodiments, the method for preparing a crystalline piperazine salt of compound 2ac comprises:
a) dissolving free form compound 2ac in acetone to form a first mixture;
b) adding to the first mixture of step a) a solution of piperazine in acetone to form a second mixture;
c) stirring the second mixture of step b) at about 25° C.;
d) after the stirring of step c), seeding the second mixture with compound 2ac piperazine salt to form a third mixture;
e) stirring the third mixture of step d) at about 25° C. to form a suspension; and
f) isolating the precipitated solids from the suspension of step e) to afford the crystalline piperazine salt of compound 2ac.
   In some embodiments of the method for preparing a crystalline piperazine salt of compound 2ac, the stirring of step c) occurs for about 1.5 hours. In some embodiments of the method for preparing a crystalline piperazine salt of compound 2ac, the stirring of step e) occurs for about 2 hours.

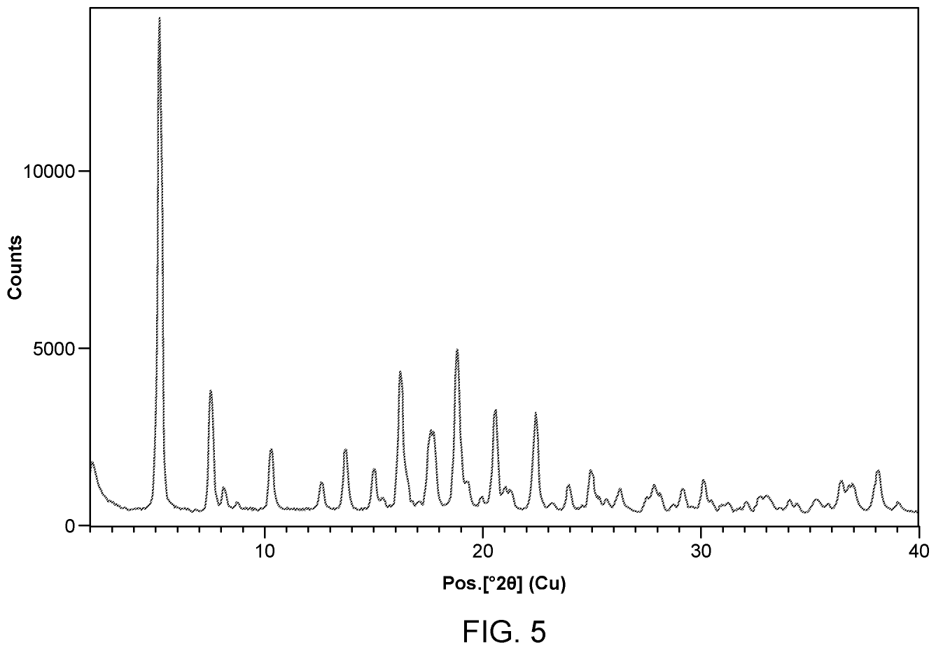

The disclosure provides a crystalline dicyclohexylamine salt of compound 2ac. In some embodiments, the crystalline dicyclohexylamine salt of compound 2ac is characterized by an XRPD pattern comprising at least one peak selected from the group consisting of 5.2, 7.5, 10.3, 13.7, 15.0, 16.2, 17.6, 20.5, 22.4, and 38.1 °2θ±0.1 °2θ. In some embodiments, the crystalline dicyclohexylamine salt of compound 2ac is characterized by an XRPD pattern comprising at least three peaks selected from the group consisting of 5.2, 7.5, 10.3, 13.7, 15.0, 16.2, 17.6, 20.5, 22.4, and 38.1 °20±0.1 °2θ. In some embodiments, the crystalline dicyclohexylamine salt of compound 2ac is characterized by an XRPD pattern comprising at least five peaks selected from the group consisting of 5.2, 7.5, 10.3, 13.7, 15.0, 16.2, 17.6, 20.5, 22.4, and 38.1 °2θ+0.1 °2θ. In some embodiments, the crystalline dicyclohexylamine salt of compound 2ac is characterized by an XRPD pattern having peaks at 5.2, 7.5, 10.3, 13.7, 15.0, 16.2, 17.6, 20.5, 22.4, and 38.1 °2θ±0.1 °2θ. In some embodiments, the crystalline dicyclohexylamine salt of compound 2ac is characterized by an XRPD pattern substantially the same as the pattern shown in FIG. 5. In some embodiments, the crystalline dicyclohexylamine salt of compound 2ac is characterized by one, two, three, four, five, six, seven, eight, nine, ten, eleven, twelve, thirteen, fourteen, fifteen, sixteen, seventeen, eighteen, nineteen, twenty, or all of the characteristic XRPD peaks as set forth in Table 9.

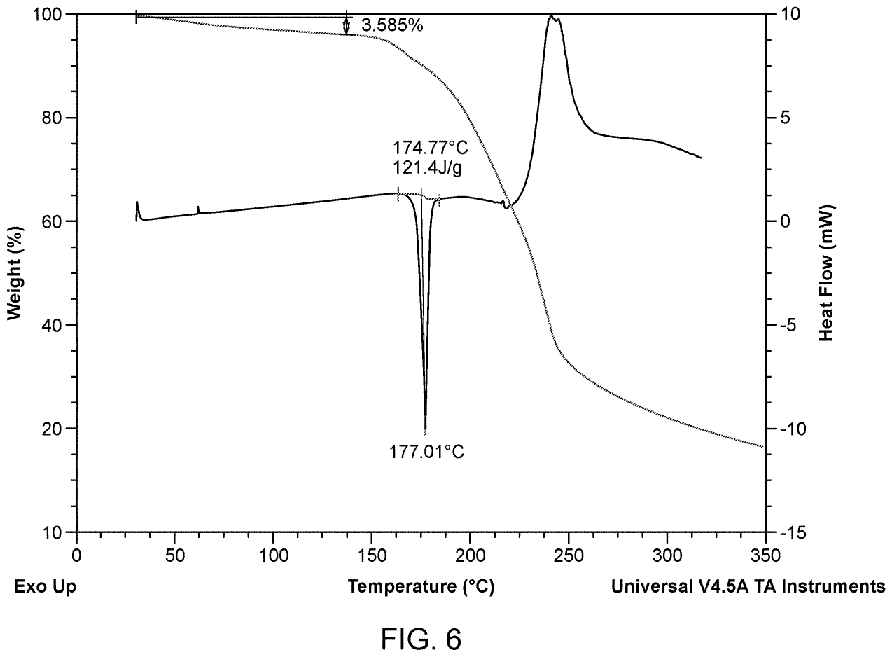

The crystalline dicyclohexylamine salt of compound 2ac may also be characterized by thermogravimetry (TG). In some embodiments, the crystalline dicyclohexylamine salt of compound 2ac is characterized by a TG thermogram indicating weight loss of about 3.58% from between about 25° C. to about 130° C. In some embodiments, the crystalline dicyclohexylamine salt of compound 2ac is characterized by a TG thermogram substantially the same as the pattern shown in FIG. 6.

The crystalline dicyclohexylamine salt of compound 2ac may also be characterized by differential scanning calorimetry (DSC). In some embodiments, the crystalline dicyclohexylamine salt of compound 2ac is characterized by a differential scanning calorimetry (DSC) thermogram comprising an endothermic peak between about 175° C. and about 179° C. In some embodiments, the crystalline dicyclohexylamine salt of compound 2ac is characterized by a DSC thermogram comprising an endothermic peak at about 177° C. In some embodiments, the crystalline dicyclohexylamine salt of compound 2ac is characterized by a DSC thermogram comprising an endothermic peak at about 177.01° C. In some embodiments, the crystalline dicyclohexylamine salt of compound 2ac is characterized by a DSC thermogram substantially the same as the pattern shown in FIG. 6.

In some embodiments, the crystalline dicyclohexylamine salt of compound 2ac is characterized by having at least two of the following:
a) an XRPD pattern comprising at least three peaks selected from the group consisting of 5.2, 7.5, 10.3, 13.7, 15.0, 16.2, 17.6, 20.5, 22.4, and 38.1 °20±0.1 °2θ;
b) an endothermic peak at about 177.01° C. as measured by DSC; and
c) weight loss of about 3.58% from between about 25° C. to about 130° C. as measured by TG.

In some embodiments, the crystalline dicyclohexylamine salt of compound 2ac is characterized by having at least two of the following:
a) an XRPD pattern substantially the same as the pattern shown in FIG. 5;
b) a TG thermogram substantially the same as the pattern shown in FIG. 6; and
c) a DSC thermogram substantially the same as the pattern shown in FIG. 6.

The disclosure also provides methods for preparing a crystalline dicyclohexylamine salt of compound 2ac. In some embodiments, the method for preparing a crystalline dicyclohexylamine salt of compound 2ac comprises:
a) dissolving free form compound 2ac in MTBE and MeOH to form a first mixture;
b) heating the first mixture of step a) to about 40° C. to about 50° C.;
c) after the heating of step b), adding dicyclohexylamine to the first mixture to form a second mixture;
d) stirring the second mixture of step c);
e) after the stirring of step d), seeding the second mixture with compound 2ac dicyclohexylamine salt to form a third mixture;
f) heating the third mixture of step e) at about 40° C. to about 50° C. to form a suspension;
g) cooling the suspension of step f) to about 15° C. to about 30° C.; and
h) isolating the precipitated solids from the suspension of step g) to afford the crystalline dicyclohexylamine salt of compound 2ac.

In some embodiments of the method for preparing a crystalline dicyclohexylamine salt of compound 2ac, the cooling of step g) is to about 25° C. In some embodiments of the method for preparing a crystalline dicyclohexylamine salt of compound 2ac, the cooling of step g) is to about 20° C. In some embodiments of the method for preparing a crystalline dicyclohexylamine salt of compound 2ac, the heating of step b) is to about 40° C. to about 45° C. In some embodiments of the method for preparing a crystalline dicyclohexylamine salt of compound 2ac, the heating of step b) is to about 45° C. In some embodiments of the method for preparing a crystalline dicyclohexylamine salt of compound 2ac, the heating of step f) is to about 40° C. to about 45° C. In some embodiments of the method for preparing a crystalline dicyclohexylamine salt of compound 2ac, the heating of step f) is to about 45° C. In some embodiments of the method for preparing a crystalline dicyclohexylamine salt of compound 2ac, the heating of step f) is for about one hour. In some embodiments of the method for preparing a crystalline dicyclohexylamine salt of compound 2ac, the heating of step f) is for about 30 minutes. In some embodiments of the method for preparing a crystalline dicyclohexylamine salt of compound 2ac, the stirring of step d) is for about 20 minutes. In some embodiments of the method for preparing a crystalline dicyclohexylamine salt of compound 2ac, the stirring of step d) is for about 30 minutes. In some embodiments of the method for preparing a crystalline dicyclohexylamine salt of compound 2ac, the cooling of step g) is for about one hour. In some embodiments of the method for preparing a crystalline dicyclohexylamine salt of compound 2ac, the adding of step c) occurs over about one hour. In some embodiments of the method for preparing a crystalline dicyclohexylamine salt of compound 2ac, additional dicyclohexylamine is added to the third mixture after step f) but before the cooling of step g). In certain such embodiments, the additional dicyclohexylamine is added to the third mixture over about four hours.

In some embodiments of the method for preparing a crystalline dicyclohexylamine salt of compound 2ac, the ratio of MTBE:MeOH in step a) is about 9:1, v/v. In some embodiments of the method for preparing a crystalline dicyclohexylamine salt of compound 2ac, the ratio of MTBE:MeOH in step a) is about 8:1, v/v. In some embodiments of the method for preparing a crystalline dicyclohexylamine salt of compound 2ac, the ratio of MTBE:MeOH in step a) is about 7:1, v/v. In some embodiments of the method for preparing a crystalline dicyclohexylamine salt of compound 2ac, the ratio of MTBE:MeOH in step a) is about 6:1, v/v. In some embodiments of the method for preparing a crystalline dicyclohexylamine salt of compound 2ac, the ratio of MTBE:MeOH in step a) is about 5:1, v/v. In some embodiments of the method for preparing a crystalline dicyclohexylamine salt of compound 2ac, the ratio of MTBE:MeOH in step a) is about 4:1, v/v. In some embodiments, the purity by HPLC (area percent (A %)) of the crystalline dicyclohexylamine salt of compound 2ac is at least 95%, at least 98%, at least 99%, or at least 99.5% pure.

The present disclosure provides salt forms of an enantiomerically enriched compound of Formula I, wherein the enantiomerically enriched compound of Formula I is compound 2ag or compound 2ah:

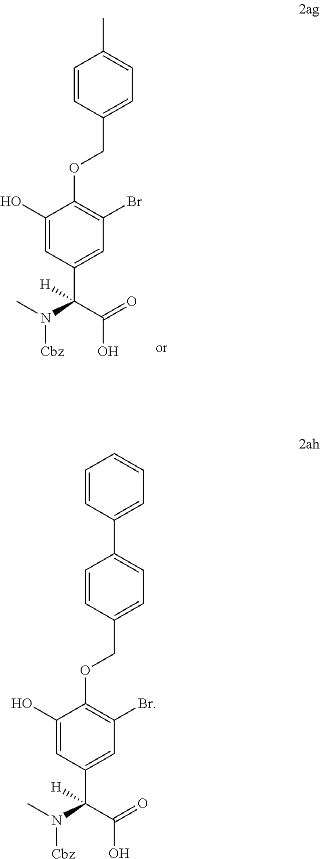 [SMILES: Cc1ccc(COc2c(O)cc([C@@H](C(=O)O)N(C)C(=O)OCc3ccccc3)cc2Br)cc1 ; CN(C(=O)OCc1ccccc1)[C@H](C(=O)O)c1cc(O)c(OCc2ccc(-c3ccccc3)cc2)c(Br)c1]

In some embodiments, the salt of compound 2ag or compound 2ah is a lysine, piperazine, or dicyclohexylamine salt. In some embodiments, the salt of compound 2ag or compound 2ah is a lysine salt. In some embodiments, the salt of compound 2ag or compound 2ah is a piperazine salt. In some embodiments, the salt of compound 2ag or compound 2ah is a dicyclohexylamine salt. In some embodiments, the disclosure provides a dicyclohexylamine salt of compound 2ag. In some embodiments, the disclosure provides a dicyclohexylamine salt of compound 2ah.

The salts of compounds 2ag or 2ah may exist as solid forms. In some embodiments, a lysine, piperazine, or dicyclohexylamine salt of compound 2ag or compound 2ah is a solid form. In some embodiments, a lysine salt of compound 2ag or compound 2ah is a solid form. In some embodiments, a piperazine salt of compound 2ag or compound 2ah is a solid form. In some embodiments, a dicyclohexylamine salt of compound 2ag or compound 2ah is a solid form.

The solid forms of the salts of compounds 2ag or 2ah may be crystalline forms. In some embodiments, a dicyclohexylamine salt of compound 2ag or compound 2ah is crystalline. In some embodiments, a lysine salt of compound 2ag or compound 2ah is crystalline. In some embodiments, a piperazine salt of compound 2ag or compound 2ah is crystalline.

The solid forms of the salts of compounds 2ag or 2ah may be amorphous solids. In some embodiments, a dicyclohexylamine salt of compound 2ag or compound 2ah is amorphous. In some embodiments, a lysine salt of compound 2ag or compound 2ah is amorphous. In some embodiments, a piperazine salt of compound 2ag or compound 2ah is amorphous.

The disclosure also provides methods for preparing a crystalline dicyclohexylamine salt of compound 2ag or compound 2ah. In some embodiments, the method for preparing a crystalline dicyclohexylamine salt of compound 2ag or compound 2ah comprises:
a) dissolving free form compound 2ag or compound 2ah in MTBE and MeOH to form a first mixture;
b) heating the first mixture of step a) to about 40° C. to about 50° C.;
c) after the heating of step b), adding dicyclohexylamine to the first mixture to form a second mixture;
d) stirring the second mixture of step c);
e) after the stirring of step d), seeding the second mixture with compound 2ag dicyclohexylamine salt or compound 2ah dicyclohexylamine salt to form a third mixture;
f) heating the third mixture of step e) at about 40° C. to about 50° C. to form a suspension;
g) cooling the suspension of step f) to about 15° C. to about 30° C.; and
h) isolating the precipitated solids from the suspension of step g) to afford the crystalline dicyclohexylamine salt of compound 2ag or compound 2ah.

In some embodiments of the method for preparing a crystalline dicyclohexylamine salt of compound 2ag or compound 2ah, the cooling of step g) is to about 25° C. In some embodiments of the method for preparing a crystalline dicyclohexylamine salt of compound 2ag or compound 2ah, the cooling of step g) is to about 20° C. In some embodiments of the method for preparing a crystalline dicyclohexylamine salt of compound 2ag or compound 2ah, the heating of step b) is to about 40° C. to about 45° C. In some embodiments of the method for preparing a crystalline dicyclohexylamine salt of compound 2ag or compound 2ah, the heating of step b) is to about 45° C. In some embodiments of the method for preparing a crystalline dicyclohexylamine salt of compound 2ag or compound 2ah, the heating of step f) is to about 40° C. to about 45° C. In some embodiments of the method for preparing a crystalline dicyclohexylamine salt of compound 2ag or compound 2ah, the heating of step f) is to about 45° C. In some embodiments of the method for preparing a crystalline dicyclohexylamine salt of compound 2ag or compound 2ah, the heating of step f) is for about one hour. In some embodiments of the method for preparing a crystalline dicyclohexylamine salt of compound 2ag or compound 2ah, the heating of step f) is for about 30 minutes. In some embodiments of the method for preparing a crystalline dicyclohexylamine salt of compound 2ag or compound 2ah, the stirring of step d) is for about 20 minutes. In some embodiments of the method for preparing a crystalline dicyclohexylamine salt of compound 2ag or compound 2ah, the stirring of step d) is for about 30 minutes. In some embodiments of the method for preparing a crystalline dicyclohexylamine salt of compound 2ag or compound 2ah, the cooling of step g) is for about one hour. In some embodiments of the method for preparing a crystalline dicyclohexylamine salt of compound 2ag or compound 2ah, the adding of step c) occurs over about one hour. In some embodiments of the method for preparing a crystalline dicyclohexylamine salt of compound 2ag or compound 2ah, additional dicyclohexylamine is added to the third mixture after step f) but before the cooling of step g). In certain such embodiments, the additional dicyclohexylamine is added to the third mixture over about four hours.

In some embodiments of the method for preparing a crystalline dicyclohexylamine salt of compound 2ag or compound 2ah, the ratio of MTBE:MeOH in step a) is about 9:1, v/v. In some embodiments of the method for preparing a crystalline dicyclohexylamine salt of compound 2ag or compound 2ah, the ratio of MTBE:MeOH in step a) is about 8:1, v/v. In some embodiments of the method for preparing a crystalline dicyclohexylamine salt of compound 2ag or compound 2ah, the ratio of MTBE:MeOH in step a) is about 7:1, v/v. In some embodiments of the method for preparing a crystalline dicyclohexylamine salt of compound 2ag or compound 2ah, the ratio of MTBE:MeOH in step a) is about 6:1, v/v. In some embodiments of the method for preparing a crystalline dicyclohexylamine salt of compound 2ag or compound 2ah, the ratio of MTBE:MeOH in step a) is about 5:1, v/v. In some embodiments of the method for preparing a crystalline dicyclohexylamine salt of compound 2ag or compound 2ah, the ratio of MTBE:MeOH in step a) is about 4:1, v/v. In some embodiments, the purity by HPLC (area percent (A %)) of the crystalline dicyclohexylamine salt of compound 2ag or compound 2ah is at least 95%, at least 98%, at least 99%, or at least 99.5% pure.

Compounds disclosed herein can be synthesized by synthetic routes that can include certain processes analogous to those well-known in the chemical arts, particularly in light of the description contained herein. Starting materials are generally available from commercial sources such as Aldrich Chemicals (Milwaukee, WI) or are readily prepared using methods well known to the skilled artisan (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Further, synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Specifically, enantiomeric mixtures of Formula I-i can be synthesized via methods generally described in Wang, W., Zheng, Z., Wang, X., Chen, J., Generation and Conjugate Additions of o-Quinone Methides Under Mild Base Conditions: Rapid Synthesis of N-Substituted Aryl Glycine Derivatives, Eur. J. Org. Chem., 8299-8306 (2013); Aurelio, L.; Box, J. S.; Brownlee, R. T. C.; Hughes, A. B.; Sleebs, M. M., An Efficient Synthesis of N-Methyl Amino Acids by Way of Intermediate 5-Oxazolidinones, J. Org. Chem. 68, 2652-2667 (2003); Emsermann, J.; Arduengo, A. J. III.; Opatz, T., Synthesis of Highly Substituted 2-¹³C-Imidazolium Salts and Metal NHC Complexes for the Investigation of Electronic Unsymmetry by NMR, Synthesis 2013, 45, 2251-2264; and Chittimalla, S. K., Kuppusamy, R., Bandi, C., A Detour Route for Meta Functionalization of Phenols, Synlett, 25, 1991-1996 (2014), each hereby incorporated by reference in its entirety.

### IV. Exemplary Embodiments

Some embodiments of the disclosure relate to Embodiment I as follows:

Embodiment I-1. A method of preparing an enantiomerically enriched compound of Formula I:

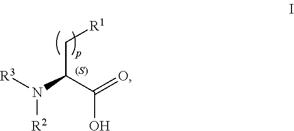

or a salt or solvate thereof,
wherein,
   R¹ is selected from the group consisting of: C₃₋₆ cycloalkyl; C₂₋₆ heterocycloalkyl having 1, 2, or 3 heteroatoms selected from O, S, and N; C₆₋₁₀ aryl; and 5- to 10-membered heteroaryl having 1, 2, or 3 heteroatoms selected from O, S, and N; wherein each can be unsubstituted or substituted with one, two, three, or four substituents independently selected from the group consisting of hydroxyl, —O-PG, —O—C₁₋₆ alkyl, halo, and C₁₋₆ alkyl,
      wherein PG is an oxygen protecting group, and
      wherein the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
         wherein R^{x} and R^{y} are each independently selected from the group consisting of hydrogen, C₁₋₆ alkyl, and a N-protecting group;
   R² is a carbamate N-protecting group;
   R³ is C₁₋₆ alkyl; and
   p is 1 or zero;
the method comprising:
   contacting in an aqueous solution an enantiomeric mixture of Formula I-i:

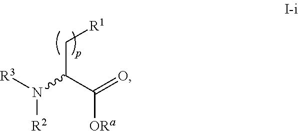

wherein,
   R¹, R², R³, and p are as defined in the compound of Formula I; and
   R^{a} is C₁₋₆ alkyl or —(CH₂CH₂O)_{q}—C₁₋₆ alkyl, wherein q is 1, 2, 3, 4, or 5;
with a carboxylic ester hydrolase to produce the enantiomerically enriched compound of Formula I, or a salt or solvate thereof.

Embodiment I-2. The method of Embodiment I-1, wherein R³ is methyl.

Embodiment I-3. The method of Embodiment I-1 or I-2, wherein R² is selected from the group consisting of Fmoc, Boc, and Cbz.

Embodiment I-4. The method of Embodiment I-3, wherein R² is Cbz.

Embodiment I-5. The method of any one of Embodiments I-1 to I-4, wherein R^{a} is C₁₋₆ alkyl.

Embodiment I-6. The method of Embodiment I-5, wherein R^{a} is n-butyl.

Embodiment I-7. The method of any one of Embodiments I-1 to I-4, wherein R^{a} is —(CH₂CH₂O)_{q}—C₁₋₆ alkyl.

Embodiment I-8. The method of Embodiment I-7, wherein R^{a} is —(CH₂CH₂O)_{q}—CH₃.

Embodiment I-9. The method of Embodiment I-7 or I-8, wherein q is 1, 2, or 3.

Embodiment I-10. The method of Embodiment I-9, wherein q is 3.

Embodiment I-11. The method of Embodiment I-9, wherein q is 2.

Embodiment I-12. The method of any one of Embodiments I-1 to I-11, wherein the carboxylic ester hydrolase is selected from the group consisting of Fluka, acylase from Streptomyces toyocaensis, 94734; Sorachim SA, cholesterol esterase from microorganism, COE-313; Toyobo Co., Ltd., lipoprotein lipase from Pseudomonas sp., LIP-301 (immobilized); Toyobo Co., Ltd., lipoprotein lipase from Pseudomonas sp., LPL-311; Sarochim SA, cholesterol esterase from Pseudomonas sp., COE-311; Amano Enzyme USA Co., Ltd., esterase from Escherichia coli, CES-E2; and Lipase 147, Evoxx Technologies GmbH, evo-1.3.147.S.

Embodiment I-13. The method of any one of Embodiments I-1 to I-12, wherein the carboxylic ester hydrolase is Lipase 147, Evoxx Technologies GmbH, evo-1.3.147.S.

Embodiment I-14. The method of any one of Embodiments I-1 to I-13, wherein the carboxylic ester hydrolase is present from about 1 wt % to about 50 wt %.

Embodiment I-15. The method of any one of Embodiments I-1 to I-14, wherein the carboxylic ester hydrolase is present from about 1 wt % to about 10 wt %.

Embodiment I-16. The method of any one of Embodiments I-1 to I-15, wherein the carboxylic ester hydrolase is present at about 5 wt %.

Embodiment I-17. The method of any one of Embodiments I-1 to I-16, wherein the aqueous solution comprises a PEG co-solvent.

Embodiment I-18. The method of Embodiment I-17, wherein the PEG co-solvent is PEG200, PEG250, PEG400, PEG4000, or PEG6000, or a combination of any of the foregoing.

Embodiment I-19. The method of Embodiment I-17 or I-18, wherein the PEG co-solvent is PEG400 or PEG200, or a combination thereof.

Embodiment I-20. The method of any one of Embodiments I-17 to I-19, wherein the PEG co-solvent is PEG400.

Embodiment I-21. The method of any one of Embodiments I-17 to I-19, wherein the PEG co-solvent is PEG200.

Embodiment I-22. The method of any one of Embodiments I-17 to I-21, wherein the PEG co-solvent is present from about 5% v/v to about 50% v/v.

Embodiment I-23. The method of Embodiment I-22, wherein the PEG co-solvent is present from about 10% v/v to about 30% v/v.

Embodiment I-24. The method of Embodiment I-22, wherein the PEG co-solvent is present at about 15% v/v to about 25% v/v.

Embodiment I-25. The method of any one of Embodiments I-1 to I-24, wherein the aqueous solution does not comprise an additional organic solvent.

Embodiment I-26. The method of any one of Embodiments I-1 to I-25, wherein the aqueous solution has a pH from about 8.0 to about 11.0.

Embodiment I-27. The method of Embodiment I-26, wherein the pH is about 8.5 to about 10.5.

Embodiment I-28. The method of any one of Embodiments I-1 to I-27, wherein the aqueous solution further comprises a buffer.

Embodiment I-29. The method of Embodiment I-28, wherein the buffer is present in an amount from about 0.1 M to about 1.5 M.

Embodiment I-30. The method of Embodiment I-29, wherein the buffer is present in an amount from about 0.5 M to about 1 M.

Embodiment I-31. The method of any one of Embodiments I-28 to I-30, wherein the buffer comprises glycine.

Embodiment I-32. The method of any one of Embodiments I-1 to I-31, wherein the contacting is at a temperature from about 30° C. to about 50° C.

Embodiment I-33. The method of Embodiment I-32, wherein the temperature is about 30° C. to about 40° C.

Embodiment I-34. The method of any one of Embodiments I-1 to I-33, wherein the concentration of the enantiomeric mixture of Formula I-i in the aqueous solution is about 10 mL/g to about 20 mL/g.

Embodiment I-35. The method of Embodiment I-34, wherein the concentration of the enantiomeric mixture of Formula I-i in the aqueous solution is about 15 mL/g.

Embodiment I-36. The method of any one of Embodiments I-1 to I-35, wherein the yield of the compound of Formula I, or a salt or solvate thereof, is at least 10%.

Embodiment I-37. The method of any one of Embodiments I-1 to I-36, wherein the yield of the compound of Formula I, or a salt or solvate thereof, is at least 20%.

Embodiment I-38. The method of any one of Embodiments I-1 to I-37, wherein the yield of the compound of Formula I, or a salt or solvate thereof, is at least 40%.

Embodiment I-39. The method of any one of Embodiments I-1 to I-38, wherein the yield of the compound of Formula I, or a salt or solvate thereof, is at least 60%.

Embodiment I-40. The method of any one of Embodiments I-1 to I-39, wherein the yield of the compound of Formula I, or a salt or solvate thereof, is at least 80%.

Embodiment I-41. The method of any one of Embodiments I-1 to I-40, wherein the yield of the compound of Formula I, or a salt or solvate thereof, is at least 90%.

Embodiment I-42. The method of any one of Embodiments I-1 to I-41, wherein the yield of the compound of Formula I, or a salt or solvate thereof, is at least 95%.

Embodiment I-43. The method of any one of Embodiments I-1 to I-42, wherein the yield of the compound of Formula I, or a salt or solvate thereof, is at least 98%.

Embodiment I-44. The method of any one of Embodiments I-1 to I-43, wherein the yield of the compound of Formula I, or a salt or solvate thereof, is at least 99%.

Embodiment I-45. The method of any one of Embodiments I-1 to I-44, wherein the compound of Formula I, or a salt or solvate thereof, has an enantiomeric excess of at least 80%.

Embodiment I-46. The method of any one of Embodiments I-1 to I-45, wherein the compound of Formula I, or a salt or solvate thereof, has an enantiomeric excess of at least 92%.

Embodiment I-47. The method of any one of Embodiments I-1 to I-46, wherein the compound of Formula I, or a salt or solvate thereof, has an enantiomeric excess of at least 94%.

Embodiment I-48. The method of any one of Embodiments I-1 to I-47, wherein the compound of Formula I, or a salt or solvate thereof, has an enantiomeric excess of at least 96%.

Embodiment I-49. The method of any one of Embodiments I-1 to I-48, wherein the compound of Formula I, or a salt or solvate thereof, has an enantiomeric excess of at least 97%.

Embodiment I-50. The method of any one of Embodiments I-1 to I-49, wherein the compound of Formula I, or a salt or solvate thereof, has an enantiomeric excess of at least 98%.

Embodiment I-51. The method of any one of Embodiments I-1 to I-50, wherein the method does not comprise chromatographic purification.

Embodiment I-52. The method of any one of Embodiments I-1 to I-51, wherein the enantiomeric mixture of Formula I-i is racemic.

Embodiment I-53. The method of any one of Embodiments I-1 to I-52, wherein the contacting is for a period of from about 1 hour to about 72 hours.

Embodiment I-54. The method of Embodiment I-53, wherein the contacting is for a period of about 24 hours to about 60 hours.

Embodiment I-55. The method of any one of Embodiments I-1 to I-54, wherein R¹ is:

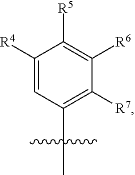

wherein, R⁴, R⁵, R⁶ and R⁷, are each independently selected from the group consisting of hydrogen, hydroxyl, —O-PG, —O—C₁₋₆ alkyl, halo, and C₁₋₆ alkyl,
   wherein PG is an oxygen protecting group, and
   wherein, the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
      wherein, R^{x} and R^{y} are each independently selected from the group consisting of hydrogen, C₁₋₆ alkyl, and a N-protecting group.

Embodiment I-56. The method of Embodiment I-55, wherein R⁴ is hydrogen or halo.

Embodiment I-57. The method of Embodiment I-56, wherein R⁴ is bromo.

Embodiment I-58. The method of Embodiment I-55, wherein R⁴ is —O-PG.

Embodiment I-59. The method of Embodiment I-58, wherein R⁴ is —O-Bn or —O-SEM.

Embodiment I-60. The method of Embodiment I-55, wherein R⁴ is unsubstituted or substituted —O—C₁₋₆ alkyl.

Embodiment I-61. The method of Embodiment I-60, wherein the —O—C₁₋₆ alkyl is substituted with hydroxyl and —NR^{x}R^{y}, wherein R^{x} is H and R^{y} is an N-protecting group.

Embodiment I-62. The method of Embodiment I-61, wherein the N-protecting group is Boc.

Embodiment I-63. The method of any one of Embodiments I-55 to I-62, wherein R⁵ is hydrogen or hydroxyl.

Embodiment I-64. The method of any one of Embodiments I-55 to I-62, wherein R⁵ is —O-PG.

Embodiment I-65. The method of Embodiment I-64, wherein R⁵ is —O-Bn or —O-SEM.

Embodiment I-66. The method of any one of Embodiments I-55 to I-65, wherein R⁶ is hydrogen, hydroxyl, halo or unsubstituted or substituted C₁₋₆ alkyl.

Embodiment I-67. The method of Embodiment I-66, wherein R⁶ is iodo, bromo, fluoro, or chloro.

Embodiment I-68. The method of Embodiment I-66, wherein R⁶ is methyl.

Embodiment I-69. The method of any one of Embodiments I-55 to I-65, wherein R⁶ is —O-PG.

Embodiment I-70. The method of Embodiment I-69, wherein R⁶ is —O-Bn or —O-SEM.

Embodiment I-71. The method of any one of Embodiments I-55 to I-70, wherein R⁷ is hydrogen, halo, or unsubstituted or substituted C₁₋₆ alkyl.

Embodiment I-72. The method of Embodiment I-71, wherein R⁷ is fluoro.

Embodiment I-73. The method of Embodiment I-71, wherein R⁷ is methyl.

Embodiment I-74. The method of Embodiment I-71, wherein R⁷ is hydrogen.

Embodiment I-75. The method of Embodiment I-55, wherein R⁴, R⁵, R⁶ and R⁷ are each hydrogen.

Embodiment I-76. The method of any one of Embodiments I-1 to I-54, wherein R¹ is an unsubstituted or substituted C₃₋₆ cycloalkyl.

Embodiment I-77. The method of Embodiment I-76, wherein R¹ is:

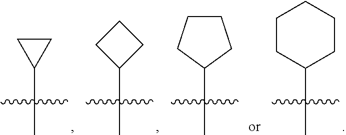

Embodiment I-78. The method of any one of Embodiments I-1 to I-54, wherein R¹ is an unsubstituted or substituted 5- to 10-membered heteroaryl having 1, 2, or 3 heteroatoms selected from O, S, and N.

Embodiment I-79. The method of Embodiment I-78, wherein R¹ is an unsubstituted or substituted pyridyl.

Embodiment I-80. The method of any one of Embodiments I-1 to I-79, wherein p is zero.

Embodiment I-81. The method of any one of Embodiments I-1 to I-79, wherein p is one.

Embodiment I-82. The method of Embodiment I-1, wherein the enantiomeric mixture of Formula I-i and the compound of Formula I, or a salt or solvate thereof, are:

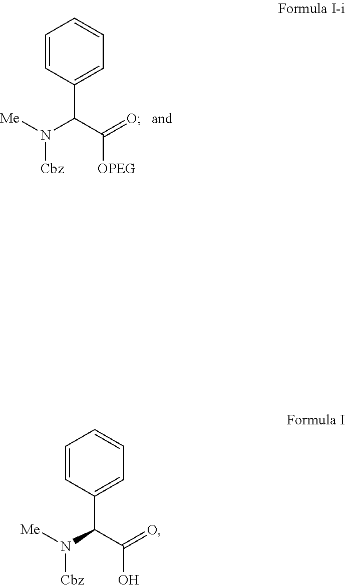

or a salt or solvate thereof; or,

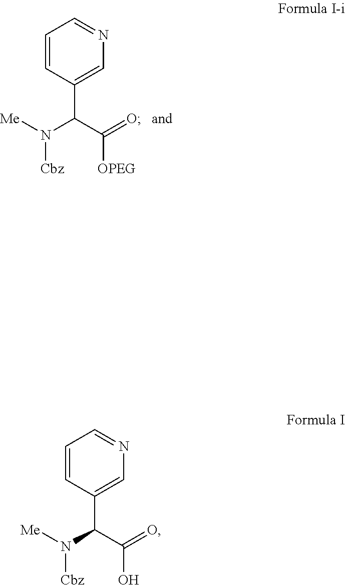

or a salt or solvate thereof; or,

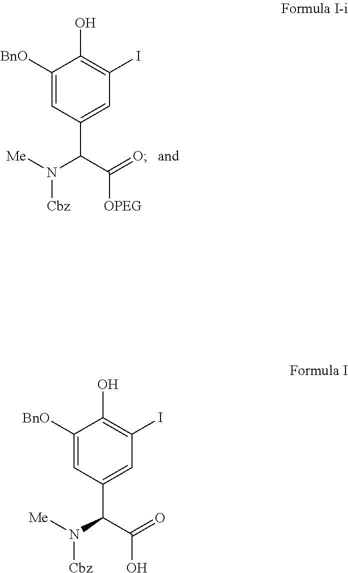

or a salt or solvate thereof; or,

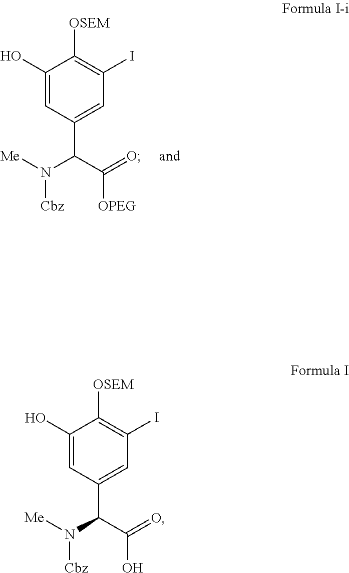

or a salt or solvate thereof; or,

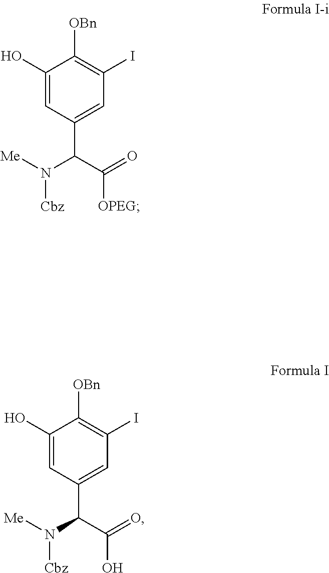

or a salt or solvate thereof; or,

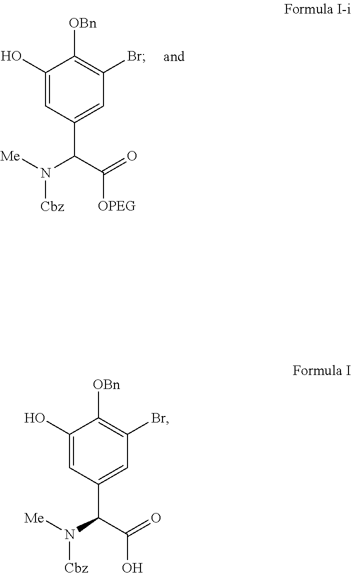

or a salt or solvate thereof; or,

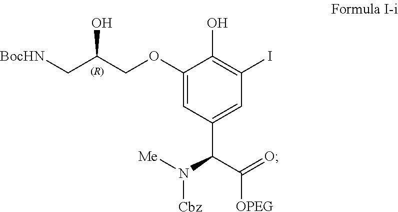

### and Formula I is

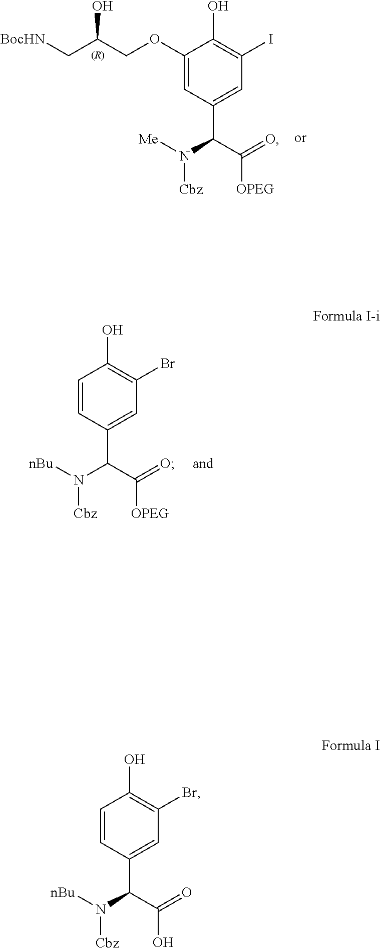

a salt or solvate thereof; or,
or a salt or solvate thereof; or,

### Formula I-i is

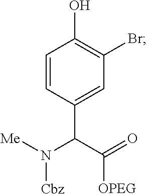

### and Formula I is

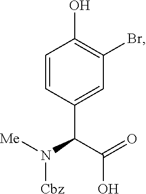 [SMILES: CN(C(=O)OCc1ccccc1)[C@H](C(=O)O)c1ccc(O)c(Br)c1]

or a salt or solvate thereof; or,

### Formula I-i is

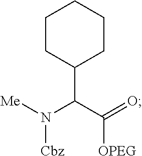

### and Formula I is

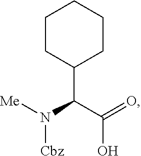 [SMILES: CN(C(=O)OCc1ccccc1)[C@H](C(=O)O)C1CCCCC1]

or a salt or solvate thereof; or,

### Formula I-i is

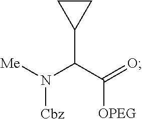

### and Formula I is

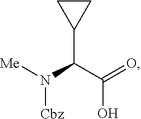 [SMILES: CN(C(=O)OCc1ccccc1)[C@H](C(=O)O)C1CC1]

or a salt or solvate thereof; or,

### Formula I-i is

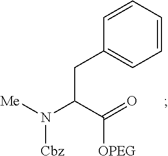

### and Formula I is

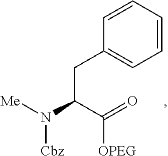

or a salt or solvate thereof; or,

### Formula I-i is

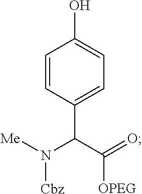

### and Formula I is

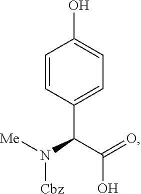 [SMILES: CN(C(=O)OCc1ccccc1)[C@H](C(=O)O)c1ccc(O)cc1]

or a salt or solvate thereof; or,

### Formula I-i is

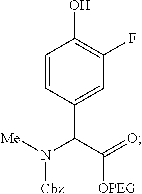

### and Formula I is

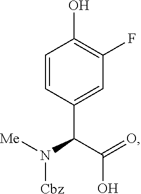 [SMILES: CN(C(=O)OCc1ccccc1)[C@H](C(=O)O)c1ccc(O)c(F)c1]

or a salt or solvate thereof; or

### Formula I-i is

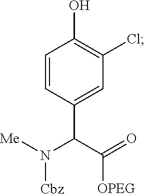

### and Formula I is

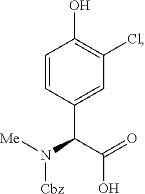 [SMILES: CN(C(=O)OCc1ccccc1)[C@H](C(=O)O)c1ccc(O)c(Cl)c1]

or a salt or solvate thereof; or,

### Formula I-i is

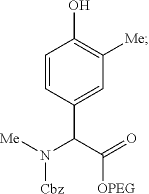

### and Formula I is

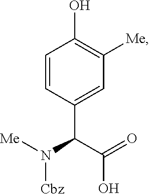 [SMILES: Cc1cc([C@@H](C(=O)O)N(C)C(=O)OCc2ccccc2)ccc1O]

or a salt or solvate thereof; or,

### Formula I-i is

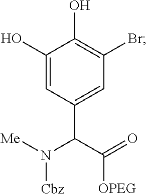

### and Formula I is

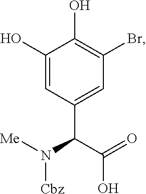 [SMILES: CN(C(=O)OCc1ccccc1)[C@H](C(=O)O)c1cc(O)c(O)c(Br)c1]

or a salt or solvate thereof; or,

### Formula I-i is

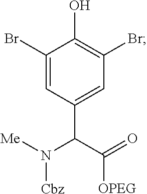

### and Formula I is

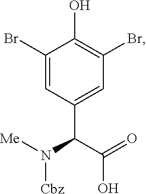 [SMILES: CN(C(=O)OCc1ccccc1)[C@H](C(=O)O)c1cc(Br)c(O)c(Br)c1]

or a salt or solvate thereof; or,

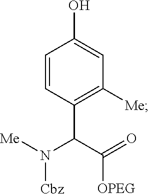

### Formula I-i is

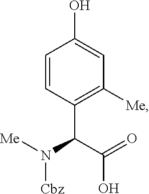 [SMILES: Cc1cc(O)ccc1[C@@H](C(=O)O)N(C)C(=O)OCc1ccccc1]

and Formula I is or a salt or solvate thereof; or,

### Formula I-i is

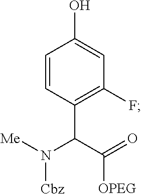

### and Formula I is

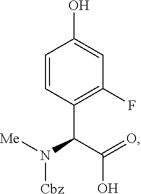 [SMILES: CN(C(=O)OCc1ccccc1)[C@H](C(=O)O)c1ccc(O)cc1F]

or a salt or solvate thereof; or,

### Formula I-i is

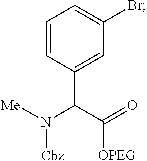

### and Formula I is

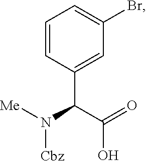 [SMILES: CN(C(=O)OCc1ccccc1)[C@H](C(=O)O)c1cccc(Br)c1]

or a salt or solvate thereof; or,

### Formula I-i is

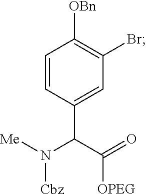

### and Formula I is

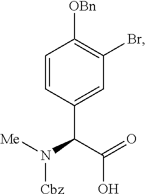 [SMILES: CN(C(=O)OCc1ccccc1)[C@H](C(=O)O)c1ccc(OCc2ccccc2)c(Br)c1]

or a salt or solvate thereof; or,

### Formula I-i is

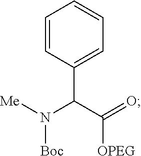

### and Formula I is

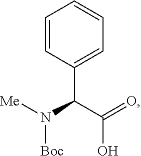 [SMILES: CN(C(=O)OC(C)(C)C)[C@H](C(=O)O)c1ccccc1]

or a salt or solvate thereof;
wherein, in each instance, PEG is —(CH₂CH₂—O)_{q}-Me, wherein q is 1, 2, or 3.

Embodiment I-83. The method of Embodiment I-82, wherein q is 1.

Embodiment I-84. The method of Embodiment I-82, wherein q is 2.

Embodiment I-85. The method of Embodiment I-82, wherein q is 3.

Embodiment I-86. The method of any one of Embodiments I-82 to I-85, wherein the enantiomeric mixture of Formula I-i and the compound of Formula I, or a salt or solvate thereof, are:

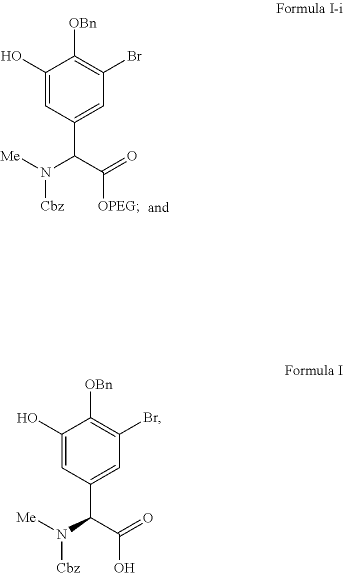

or a salt or solvate thereof.

Embodiment I-87. The method of Embodiment I-1, wherein the enantiomeric mixture of Formula I-i and the compound of Formula I, or a salt or solvate thereof, are:

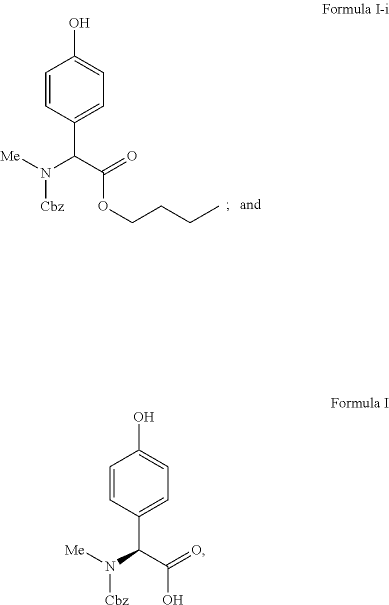 [SMILES: CCCCOC(=O)C(c1ccc(O)cc1)N(C)C(=O)OCc1ccccc1 ; CN(C(=O)OCc1ccccc1)[C@H](C(=O)O)c1ccc(O)cc1]

or a salt or solvate thereof; or,

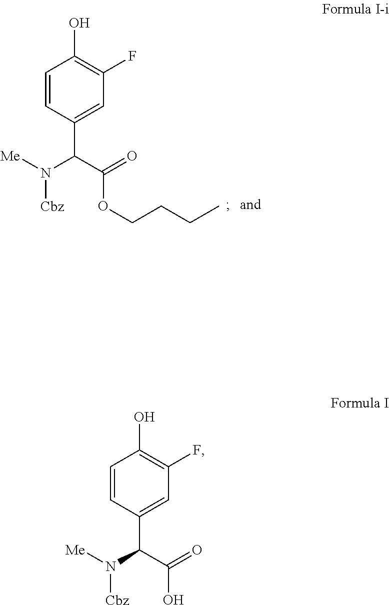 [SMILES: CCCCOC(=O)C(c1ccc(O)c(F)c1)N(C)C(=O)OCc1ccccc1 ; CN(C(=O)OCc1ccccc1)[C@H](C(=O)O)c1ccc(O)c(F)c1]

or a salt or solvate thereof; or,

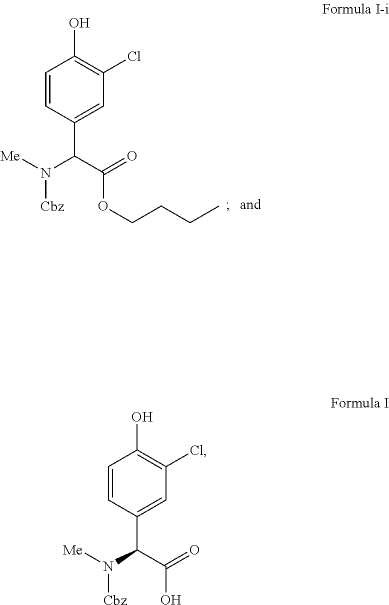 [SMILES: CCCCOC(=O)C(c1ccc(O)c(Cl)c1)N(C)C(=O)OCc1ccccc1 ; CN(C(=O)OCc1ccccc1)[C@H](C(=O)O)c1ccc(O)c(Cl)c1]

or a salt or solvate thereof; or,

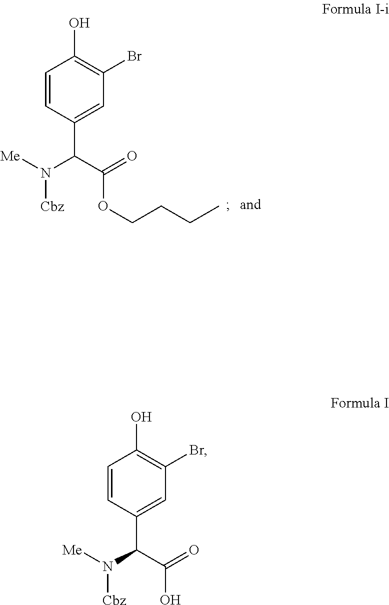 [SMILES: CCCCOC(=O)C(c1ccc(O)c(Br)c1)N(C)C(=O)OCc1ccccc1 ; CN(C(=O)OCc1ccccc1)[C@H](C(=O)O)c1ccc(O)c(Br)c1]

or a salt or solvate thereof; or,

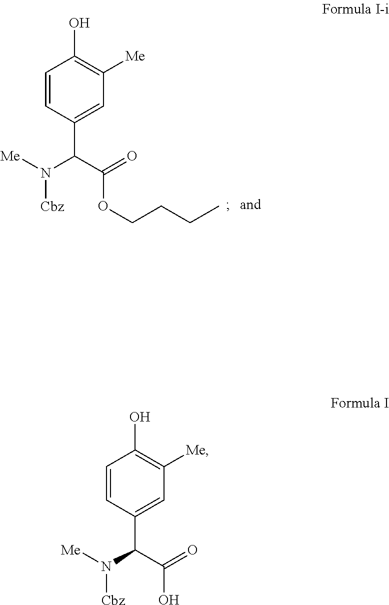 [SMILES: CCCCOC(=O)C(c1ccc(O)c(C)c1)N(C)C(=O)OCc1ccccc1 ; Cc1cc([C@@H](C(=O)O)N(C)C(=O)OCc2ccccc2)ccc1O]

or a salt or solvate thereof; or,

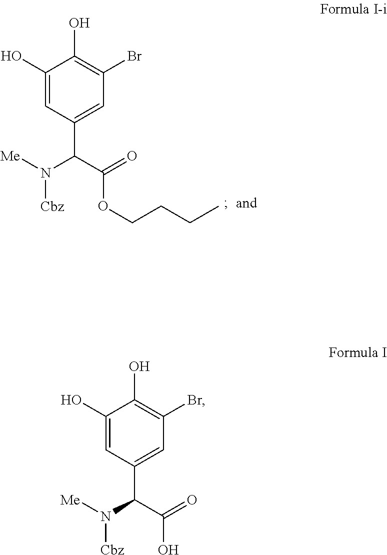 [SMILES: CCCCOC(=O)C(c1cc(O)c(O)c(Br)c1)N(C)C(=O)OCc1ccccc1 ; CN(C(=O)OCc1ccccc1)[C@H](C(=O)O)c1cc(O)c(O)c(Br)c1]

or a salt or solvate thereof; or,

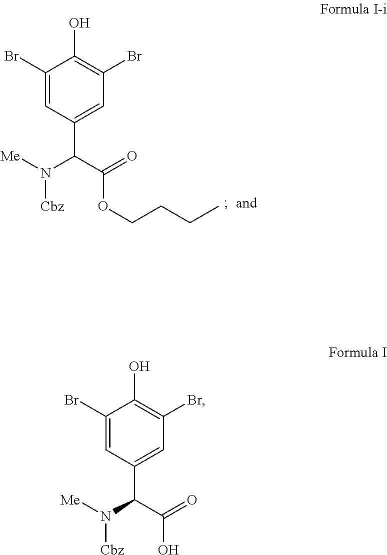 [SMILES: CCCCOC(=O)C(c1cc(Br)c(O)c(Br)c1)N(C)C(=O)OCc1ccccc1 ; CN(C(=O)OCc1ccccc1)[C@H](C(=O)O)c1cc(Br)c(O)c(Br)c1]

or a salt or solvate thereof; or,

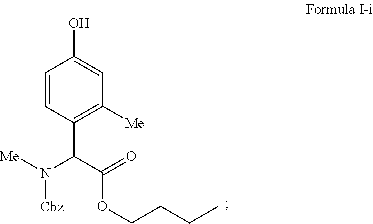 [SMILES: CCCCOC(=O)C(c1ccc(O)cc1C)N(C)C(=O)OCc1ccccc1]

### and Formula I is

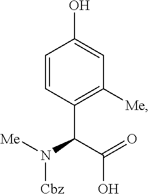 [SMILES: Cc1cc(O)ccc1[C@@H](C(=O)O)N(C)C(=O)OCc1ccccc1]

or a salt or solvate thereof; or,

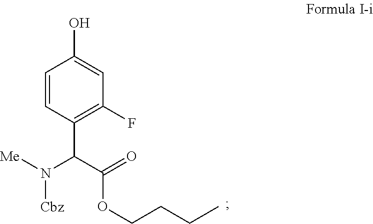 [SMILES: CCCCOC(=O)C(c1ccc(O)cc1F)N(C)C(=O)OCc1ccccc1]

### and Formula I is

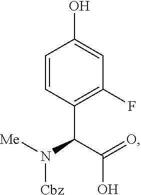 [SMILES: CN(C(=O)OCc1ccccc1)[C@H](C(=O)O)c1ccc(O)cc1F]

or a salt or solvate thereof; or,

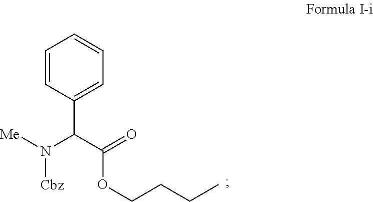 [SMILES: CCCCOC(=O)C(c1ccccc1)N(C)C(=O)OCc1ccccc1]

### and Formula I is

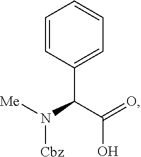 [SMILES: CN(C(=O)OCc1ccccc1)[C@H](C(=O)O)c1ccccc1]

or a salt or solvate thereof; or,

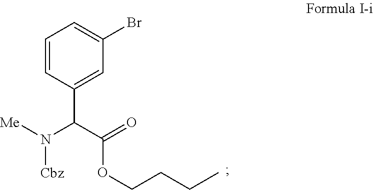 [SMILES: CCCCOC(=O)C(c1cccc(Br)c1)N(C)C(=O)OCc1ccccc1]

### and Formula I is

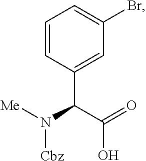 [SMILES: CN(C(=O)OCc1ccccc1)[C@H](C(=O)O)c1cccc(Br)c1]

or a salt or solvate thereof; or,

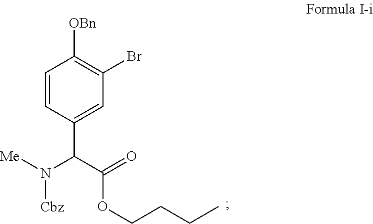 [SMILES: CCCCOC(=O)C(c1ccc(OCc2ccccc2)c(Br)c1)N(C)C(=O)OCc1ccccc1]

### and Formula I is

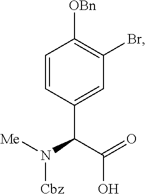 [SMILES: CN(C(=O)OCc1ccccc1)[C@H](C(=O)O)c1ccc(OCc2ccccc2)c(Br)c1]

or a salt or solvate thereof.

Embodiment I-88. The method of Embodiment I-1, wherein the enantiomerically enriched compound of Formula I is:

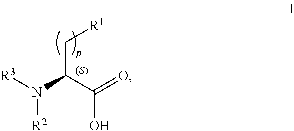

or a salt or solvate thereof,
wherein,
   R¹ is selected from the group consisting of: C₃₋₆ cycloalkyl and phenyl, wherein each can be unsubstituted or substituted with one, two, three, or four substituents independently selected from the group consisting of hydroxyl, fluoro, chloro, bromo, and methyl;
   R² is a carbamate N-protecting group;
   R³ is methyl; and
   p is 1 or zero;
      the method comprising:
contacting in an aqueous solution, wherein the aqueous solution comprises PEG400, at a temperature of about 45° C., an enantiomeric mixture of Formula I-i:

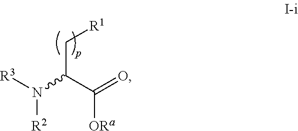

wherein,
   R¹, R², R³, and p are as defined in the compound of Formula I; and
   R^{a} is n-butyl or —(CH₂CH₂O)_{q}-Me, wherein q is 2 or 3;
with a solution of Lipase 147, Evoxx Technologies GmbH, evo-1.3.147.S dissolved in aqueous glycine buffer at a pH from about 9.0 to about 10.0, to produce the enantiomerically enriched compound of Formula I, or a salt or solvate thereof.

Embodiment I-89. The method of Embodiment I-88, wherein R^{a} is —(CH₂CH₂O)_{q}-Me.

Embodiment I-90. The method of Embodiment I-89, wherein q is 2.

Embodiment I-91. The method of Embodiment I-89, wherein q is 3.

Embodiment I-92. The method of Embodiment I-88, wherein R^{a} is n-butyl.

Embodiment I-93. The method of any one of Embodiments I-88 to I-92, wherein R² is Cbz.

Embodiment I-94. The method of any one of Embodiments I-88 to I-93, wherein R¹ is unsubstituted or substituted C₃₋₆ cycloalkyl.

Embodiment I-95. The method of any one of Embodiments I-88 to I-93, wherein R¹ is unsubstituted or substituted phenyl.

Embodiment I-96. The method of any one of Embodiments I-1 to I-95, wherein the contacting is under dynamic kinetic resolution conditions.

Embodiment I-97. The method of any one of Embodiments I-1 to I-96, further comprising:
a) adding a first organic solvent to an aqueous solution comprising the produced enantiomerically enriched compound of Formula I to form a first mixture;
b) separating the first mixture into a first aqueous phase and a first organic phase;
c) adjusting the pH of the first aqueous phase to about 7.0;
d) adding a second organic solvent to the first aqueous phase to form a second mixture;
e) adjusting the pH of the second mixture to about 2.0 to about 3.0;
f) separating the second mixture into a second aqueous phase and second organic phase;
and g) concentrating the second organic phase,
   to prepare the enantiomerically enriched compound of Formula I, or a salt or solvate thereof.

Embodiment I-98. The method of any one of Embodiments I-1 to I-96, further comprising:
a) adding a first organic solvent to an aqueous solution comprising the produced enantiomerically enriched compound of Formula I to form a first mixture;
b) separating the first mixture into a first aqueous phase and a first organic phase;
c) adding a second organic solvent to the first aqueous phase to form a second mixture;
d) adjusting the pH of the second mixture to below about 7.0;
e) separating the second mixture into a second aqueous phase and second organic phase; and
f) concentrating the second organic phase,
   to prepare the enantiomerically enriched compound of Formula I, or a salt or solvate thereof.

Embodiment I-99. The method of Embodiment I-97 or I-98, wherein the first organic solvent is isopropyl acetate, cyclohexanes, or MTBE, or a combination of any of the foregoing.

Embodiment I-100. The method of any one of Embodiments I-97 to I-99, wherein the second organic solvent is isopropyl acetate, cyclohexanes, or MTBE, or a combination of any of the foregoing.

Embodiment I-101. The method of any one of Embodiments I-97 to I-100, wherein step a) further comprises adding water to the aqueous solution.

Embodiment I-102. The method of any one of Embodiments I-1 to I-101, further comprising isolating the enantiomerically enriched compound of Formula I, or a salt or solvate thereof.

Embodiment I-103. The method of any one of Embodiments I-1 to I-102, wherein the enantiomerically enriched compound of Formula I is a salt.

Embodiment I-104. The method of Embodiment I-103, wherein the salt of the enantiomerically enriched compound of Formula I is a lysine, piperazine, or dicyclohexylamine salt.

Embodiment I-105. The method of Embodiment I-103 or I-104, wherein the salt of the enantiomerically enriched compound of Formula I is crystalline.

Embodiment I-106. A lysine salt of compound 2ac:

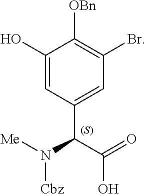

Embodiment I-107. The lysine salt of Embodiment I-106, wherein the lysine salt is crystalline.

Embodiment I-108. The lysine salt of Embodiment I-106 or I-107, characterized by an XRPD pattern comprising at least five peaks selected from the group consisting of 3.8, 6.9, 13.3, 13.9, 20.9, 22.0, 22.4, and 25.2 °2θ±0.1 °2θ.

Embodiment I-109. The lysine salt of any one of Embodiments I-106 to I-108, characterized by an XRPD pattern substantially the same as the pattern shown in FIG. 1.

Embodiment I-110. The lysine salt of any one of Embodiments I-106 to I-109, characterized by a thermogravimetric (TG) thermogram indicating continuous weight loss of about 4.09% from between about 18.9° C. to about 150° C.

Embodiment I-111. The lysine salt of any one of Embodiments I-106 to I-110, characterized by a TG thermogram substantially the same as the pattern shown in FIG. 2.

Embodiment I-112. The lysine salt of any one of Embodiments I-106 to I-111, characterized by a differential scanning calorimetry (DSC) thermogram comprising endothermic peak between about 133° C. and about 137° C. and an endothermic peak between about 192° C. and about 196° C.

Embodiment I-113. The lysine salt of any one of Embodiments I-106 to I-112, characterized by a DSC thermogram comprising an endothermic peak at about 134.6° C. and an endothermic peak at about 194.4° C.

Embodiment I-114. The lysine salt of any one of Embodiments I-106 to I-113, characterized by a DSC thermogram substantially the same as the pattern shown in FIG. 2.

Embodiment I-115. The lysine salt of any one of Embodiments I-106 to I-114 prepared by the method of any one of Embodiments I-1, I-82, I-86, or I-88.

Embodiment I-116. A piperazine salt of compound 2ac:

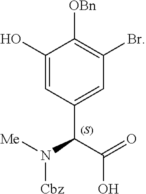

Embodiment I-117. The piperazine salt of Embodiment I-116, wherein the piperazine salt is crystalline.

Embodiment I-118. The piperazine salt of Embodiment I-116 or I-117, characterized by an XRPD pattern comprising at least two peaks selected from the group consisting of 11.0, 12.6, and 19.0 °2θ±0.1 °2θ.

Embodiment I-119. The piperazine salt of any one of Embodiments I-116 to I-118, characterized by an XRPD pattern substantially the same as the pattern shown in FIG. 3.

Embodiment I-120. The piperazine salt of any one of Embodiments I-116 to I-119, characterized by a thermogravimetric (TG) thermogram indicating continuous weight loss of about 4.93% from between about 27.6° C. to about 100° C.

Embodiment I-121. The piperazine salt of any one of Embodiments I-116 to I-120, characterized by a TG thermogram substantially the same as the pattern shown in FIG. 4.

Embodiment I-122. The piperazine salt of any one of Embodiments I-116 to I-121, characterized by a differential scanning calorimetry (DSC) thermogram comprising an endothermic peak between about 88° C. and about 92° C.

Embodiment I-123. The piperazine salt of any one of Embodiments I-116 to I-122, characterized by a DSC thermogram comprising an endothermic peak at about 89.8° C.

Embodiment I-124. The piperazine salt of any one of Embodiments I-116 to I-123, characterized by a DSC thermogram substantially the same as the pattern shown in FIG. 4.

Embodiment I-125. The piperazine salt of any one of Embodiments I-116 to I-124 prepared by the method of any one of Embodiments I-1, I-82, I-86, or I-88.

Embodiment I-126. A dicyclohexylamine salt of compound 2ac:

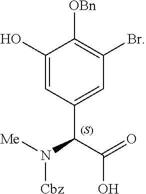

Embodiment I-127. The dicyclohexylamine salt of Embodiment I-126, wherein the dicyclohexylamine salt is crystalline.

Embodiment I-128. The dicyclohexylamine salt of Embodiment I-126 or I-127, characterized by an XRPD pattern comprising at least five peaks selected from the group consisting of 5.2, 7.5, 10.3, 13.7, 15.0, 16.2, 17.6, 20.5, 22.4, and 38.1 °20±0.1 °2θ.

Embodiment I-129. The dicyclohexylamine salt of any one of Embodiments I-126 to I-128, characterized by an XRPD pattern substantially the same as the pattern shown in FIG. 5.

Embodiment I-130. The dicyclohexylamine salt of any one of Embodiments I-126 to I-129, characterized by a thermogravimetric (TG) thermogram indicating continuous weight loss of about 3.58% between about 25° C. and about 130° C.

Embodiment I-131. The dicyclohexylamine salt of any one of Embodiments I-126 to I-130, characterized by a TG thermogram substantially the same as the pattern shown in FIG. 6.

Embodiment I-132. The dicyclohexylamine salt of any one of Embodiments I-126 to I-131, characterized by a differential scanning calorimetry (DSC) thermogram comprising an endothermic peak between about 175° C. and about 179° C.

Embodiment I-133. The dicyclohexylamine salt of any one of Embodiments I-126 to I-132, characterized by a DSC thermogram comprising an endothermic peak at about 177.01° C.

Embodiment I-134. The dicyclohexylamine salt of any one of Embodiments I-126 to I-133, characterized by a DSC thermogram substantially the same as the pattern shown in FIG. 6.

Embodiment I-135. The dicyclohexylamine salt of any one of Embodiments I-126 to I-134 prepared by the method of any one of Embodiments I-1, I-82, I-86, or I-88.

Some embodiments of the disclosure relate to Embodiment II as follows:

Embodiment II-1. A method of preparing an enantiomerically enriched compound of Formula I:

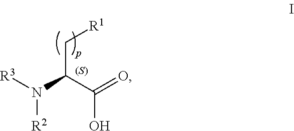

or a salt or solvate thereof,
wherein,
   R¹ is selected from the group consisting of: C₃₋₆ cycloalkyl; C₂₋₆ heterocycloalkyl having 1, 2, or 3 heteroatoms selected from O, S, and N; C₆₋₁₀ aryl; and 5- to 10-membered heteroaryl having 1, 2, or 3 heteroatoms selected from O, S, and N; wherein each can be unsubstituted or substituted with one, two, three, or four substituents independently selected from the group consisting of hydroxyl, —O-PG, —O—C₁₋₆ alkyl, halo, and C₁₋₆ alkyl,
      wherein PG is an oxygen protecting group, and
      wherein the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
      wherein R^{x} and R^{y} are each independently selected from the group consisting of hydrogen, C₁₋₆ alkyl, and a N-protecting group;
   R² is a carbamate N-protecting group;
   R³ is C₁₋₆ alkyl; and
   p is 1 or zero;
the method comprising:
   contacting in an aqueous solution an enantiomeric mixture of Formula I-i:

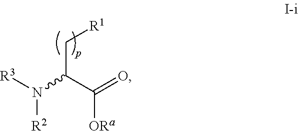

wherein,
   R¹, R², R³, and p are as defined in the compound of Formula I; and
   R^{a} is C₁₋₆ alkyl or —(CH₂CH₂O)_{q}—C₁₋₆ alkyl, wherein q is 1, 2, 3, 4, or 5;
with a carboxylic ester hydrolase to produce the enantiomerically enriched compound of Formula I, or a salt or solvate thereof.

Embodiment II-2. The method of Embodiment II-1, wherein R³ is methyl.

Embodiment II-3. The method of Embodiment II-1 or II-2, wherein R² is selected from the group consisting of Fmoc, Boc, and Cbz.

Embodiment II-4. The method of Embodiment II-3, wherein R² is Cbz.

Embodiment II-5. The method of any one of Embodiments II-1 to II-4, wherein R^{a} is C₁₋₆ alkyl.

Embodiment II-6. The method of Embodiment II-5, wherein R^{a} is n-butyl.

Embodiment II-7. The method of any one of Embodiments II-1 to II-4, wherein R^{a} is —(CH₂CH₂O)_{q}—C₁₋₆ alkyl.

Embodiment II-8. The method of Embodiment II-7, wherein R^{a} is —(CH₂CH₂O)_{q}—CH₃.

Embodiment II-9. The method of Embodiment II-7 or II-8, wherein q is 1, 2, or 3.

Embodiment II-10. The method of Embodiment II-9, wherein q is 3.

Embodiment II-11. The method of Embodiment II-9, wherein q is 2.

Embodiment II-12. The method of any one of Embodiments II-1 to II-11, wherein the carboxylic ester hydrolase is selected from the group consisting of Fluka, acylase from Streptomyces toyocaensis, 94734; Sorachim SA, cholesterol esterase from microorganism, COE-313; Toyobo Co., Ltd., lipoprotein lipase from Pseudomonas sp., LIP-301 (immobilized); Toyobo Co., Ltd., lipoprotein lipase from Pseudomonas sp., LPL-311; Sarochim SA, cholesterol esterase from Pseudomonas sp., COE-311; Amano Enzyme USA Co., Ltd., esterase from Escherichia coli, CES-E2; and Lipase 147, Evoxx Technologies GmbH, evo-1.3.147.S.

Embodiment II-13. The method of any one of Embodiments II-1 to II-12, wherein the carboxylic ester hydrolase is Lipase 147, Evoxx Technologies GmbH, evo-1.3.147.S.

Embodiment II-14. The method of any one of Embodiments II-1 to II-13, wherein the carboxylic ester hydrolase is present from about 1 wt % to about 50 wt %.

Embodiment II-15. The method of any one of Embodiments II-1 to II-14, wherein the carboxylic ester hydrolase is present from about 1 wt % to about 10 wt %.

Embodiment II-16. The method of any one of Embodiments II-1 to II-15, wherein the carboxylic ester hydrolase is present at about 5 wt %.

Embodiment II-17. The method of any one of Embodiments II-1 to II-16, wherein the aqueous solution comprises a PEG co-solvent.

Embodiment II-18. The method of Embodiment II-17, wherein the PEG co-solvent is PEG200, PEG250, PEG400, PEG4000, or PEG6000, or a combination of any of the foregoing.

Embodiment II-19. The method of Embodiment II-17 or II-18, wherein the PEG co-solvent is PEG400 or PEG200, or a combination thereof.

Embodiment II-20. The method of any one of Embodiments II-17 to II-19, wherein the PEG co-solvent is PEG400.

Embodiment II-21. The method of any one of Embodiments II-17 to II-19, wherein the PEG co-solvent is PEG200.

Embodiment II-22. The method of any one of Embodiments II-17 to II-21, wherein the PEG co-solvent is present from about 5% v/v to about 50% v/v.

Embodiment II-23. The method of Embodiment II-22, wherein the PEG co-solvent is present from about 10% v/v to about 30% v/v.

Embodiment II-24. The method of Embodiment II-22, wherein the PEG co-solvent is present at about 15% v/v to about 25% v/v.

Embodiment II-25. The method of any one of Embodiments II-1 to II-24, wherein the aqueous solution does not comprise an additional organic solvent.

Embodiment II-26. The method of any one of Embodiments II-1 to II-25, wherein the aqueous solution has a pH from about 8.0 to about 11.0.

Embodiment II-27. The method of Embodiment II-26, wherein the pH is about 8.5 to about 10.5.

Embodiment II-28. The method of any one of Embodiments II-1 to II-27, wherein the aqueous solution further comprises a buffer.

Embodiment II-29. The method of Embodiment II-28, wherein the buffer is present in an amount from about 0.1 M to about 1.5 M.

Embodiment II-30. The method of Embodiment II-29, wherein the buffer is present in an amount from about 0.5 M to about 1 M.

Embodiment II-31. The method of any one of Embodiments II-28 to II-30, wherein the buffer comprises glycine.

Embodiment II-32. The method of any one of Embodiments II-1 to II-31, wherein the contacting is at a temperature from about 30° C. to about 50° C.

Embodiment II-33. The method of Embodiment II-32, wherein the temperature is about 30° C. to about 40° C.

Embodiment II-34. The method of any one of Embodiments II-1 to II-33, wherein the concentration of the enantiomeric mixture of Formula I-i in the aqueous solution is about 10 mL/g to about 20 mL/g.

Embodiment II-35. The method of Embodiment II-34, wherein the concentration of the enantiomeric mixture of Formula I-i in the aqueous solution is about 15 mL/g.

Embodiment II-36. The method of any one of Embodiments II-1 to II-35, wherein the yield of the compound of Formula I, or a salt or solvate thereof, is at least 10%.

Embodiment II-37. The method of any one of Embodiments II-1 to II-36, wherein the yield of the compound of Formula I, or a salt or solvate thereof, is at least 20%.

Embodiment II-38. The method of any one of Embodiments II-1 to II-37, wherein the yield of the compound of Formula I, or a salt or solvate thereof, is at least 40%.

Embodiment II-39. The method of any one of Embodiments II-1 to II-38, wherein the yield of the compound of Formula I, or a salt or solvate thereof, is at least 60%.

Embodiment II-40. The method of any one of Embodiments II-1 to II-39, wherein the yield of the compound of Formula I, or a salt or solvate thereof, is at least 80%.

Embodiment II-41. The method of any one of Embodiments II-1 to II-40, wherein the yield of the compound of Formula I, or a salt or solvate thereof, is at least 90%.

Embodiment II-42. The method of any one of Embodiments II-1 to II-41, wherein the yield of the compound of Formula I, or a salt or solvate thereof, is at least 95%.

Embodiment II-43. The method of any one of Embodiments II-1 to II-42, wherein the yield of the compound of Formula I, or a salt or solvate thereof, is at least 98%.

Embodiment II-44. The method of any one of Embodiments II-1 to II-43, wherein the yield of the compound of Formula I, or a salt or solvate thereof, is at least 99%.

Embodiment II-45. The method of any one of Embodiments II-1 to II-44, wherein the compound of Formula I, or a salt or solvate thereof, has an enantiomeric excess of at least 80%.

Embodiment II-46. The method of any one of Embodiments II-1 to II-45, wherein the compound of Formula I, or a salt or solvate thereof, has an enantiomeric excess of at least 92%.

Embodiment II-47. The method of any one of Embodiments II-1 to II-46, wherein the compound of Formula I, or a salt or solvate thereof, has an enantiomeric excess of at least 94%.

Embodiment II-48. The method of any one of Embodiments II-1 to II-47, wherein the compound of Formula I, or a salt or solvate thereof, has an enantiomeric excess of at least 96%.

Embodiment II-49. The method of any one of Embodiments II-1 to II-48, wherein the compound of Formula I, or a salt or solvate thereof, has an enantiomeric excess of at least 97%.

Embodiment II-50. The method of any one of Embodiments II-1 to II-49, wherein the compound of Formula I, or a salt or solvate thereof, has an enantiomeric excess of at least 98%.

Embodiment II-51. The method of any one of Embodiments II-1 to II-50, wherein the method does not comprise chromatographic purification.

Embodiment II-52. The method of any one of Embodiments II-1 to II-51, wherein the enantiomeric mixture of Formula I-i is racemic.

Embodiment II-53. The method of any one of Embodiments II-1 to II-52, wherein the contacting is for a period of from about 1 hour to about 72 hours.

Embodiment II-54. The method of Embodiment II-53, wherein the contacting is for a period of about 24 hours to about 60 hours.

Embodiment II-55. The method of any one of Embodiments II-1 to II-54, wherein R¹ is:

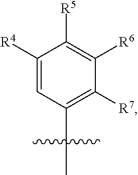

wherein, R⁴, R⁵, R⁶ and R⁷, are each independently selected from the group consisting of hydrogen, hydroxyl, —O-PG, —O—C₁₋₆ alkyl, halo, and C₁₋₆ alkyl,
   wherein PG is an oxygen protecting group, and
   wherein, the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
      wherein, R^{x} and R^{y} are each independently selected from the group consisting of hydrogen, C₁₋₆ alkyl, and a N-protecting group.

Embodiment II-56. The method of Embodiment II-55, wherein R⁴ is hydrogen or halo.

Embodiment II-57. The method of Embodiment II-56, wherein R⁴ is bromo.

Embodiment II-58. The method of Embodiment II-55, wherein R⁴ is —O-PG.

Embodiment II-59. The method of Embodiment II-58, wherein R⁴ is —O-Bn or —O-SEM.

Embodiment II-60. The method of Embodiment II-55, wherein R⁴ is unsubstituted or substituted —O—C₁₋₆ alkyl.

Embodiment II-61. The method of Embodiment II-60, wherein the —O—C₁₋₆ alkyl is substituted with hydroxyl and —NR^{x}R^{y}, wherein R^{x} is H and R^{y} is an N-protecting group.

Embodiment II-62. The method of Embodiment II-61, wherein the N-protecting group is Boc.

Embodiment II-63. The method of any one of Embodiments II-55 to II-62, wherein R⁵ is hydrogen or hydroxyl.

Embodiment II-64. The method of any one of Embodiments II-55 to II-62, wherein R⁵ is —O-PG.

Embodiment II-65. The method of Embodiment II-64, wherein R⁵ is —O-Bn or —O-SEM.

Embodiment II-66. The method of Embodiment II-64, wherein R⁵ is —O-p-methylbenzyl.

Embodiment II-67. The method of Embodiment II-64, wherein R⁵ is —O-p-phenylbenzyl.

Embodiment II-68. The method of any one of Embodiments II-55 to II-67, wherein R⁶ is hydrogen, hydroxyl, halo or unsubstituted or substituted C₁₋₆ alkyl.

Embodiment II-69. The method of Embodiment II-68, wherein R⁶ is iodo, bromo, fluoro, or chloro.

Embodiment II-70. The method of Embodiment II-68, wherein R⁶ is methyl.

Embodiment II-71. The method of any one of Embodiments II-55 to II-67, wherein R⁶ is —O-PG.

Embodiment II-72. The method of Embodiment II-71, wherein R⁶ is —O-Bn or —O-SEM.

Embodiment II-73. The method of any one of Embodiments II-55 to II-72, wherein

R⁷ is hydrogen, halo, or unsubstituted or substituted C₁₋₆ alkyl.

Embodiment II-74. The method of Embodiment II-73, wherein R⁷ is fluoro.

Embodiment II-75. The method of Embodiment II-73, wherein R⁷ is methyl.

Embodiment II-76. The method of Embodiment II-73, wherein R⁷ is hydrogen.

Embodiment II-77. The method of Embodiment II-55, wherein R⁴, R⁵, R⁶ and R⁷ are each hydrogen.

Embodiment II-78. The method of any one of Embodiments II-1 to II-54, wherein R¹ is an unsubstituted or substituted C₃₋₆ cycloalkyl.

Embodiment II-79. The method of Embodiment II-78, wherein R¹ is:

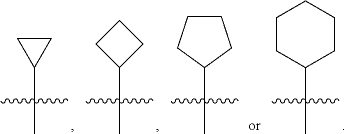

Embodiment II-80. The method of any one of Embodiments II-1 to II-54, wherein R¹ is an unsubstituted or substituted 5- to 10-membered heteroaryl having 1, 2, or 3 heteroatoms selected from O, S, and N.

Embodiment II-81. The method of Embodiment II-80, wherein R¹ is an unsubstituted or substituted pyridyl.

Embodiment II-82. The method of any one of Embodiments II-1 to II-81, wherein p is zero.

Embodiment II-83. The method of any one of Embodiments II-1 to II-81, wherein p is one.

Embodiment II-84. The method of Embodiment II-1, wherein the enantiomeric mixture of Formula I-i and the compound of Formula I, or a salt or solvate thereof, are:

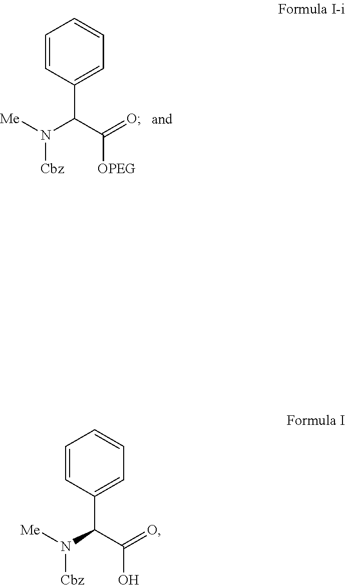

or a salt or solvate thereof; or,

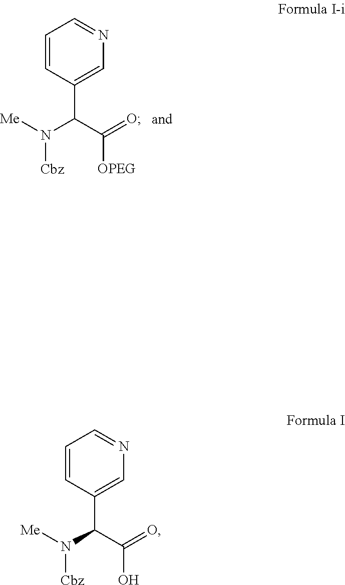

or a salt or solvate thereof; or,

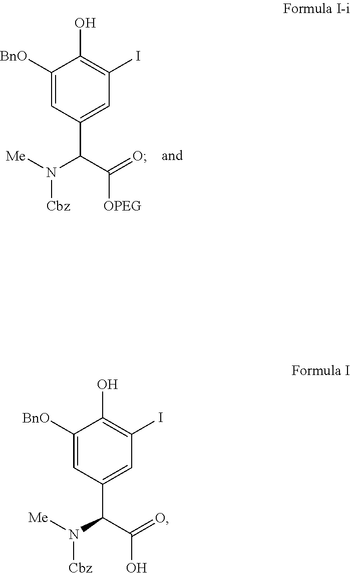

or a salt or solvate thereof; or,

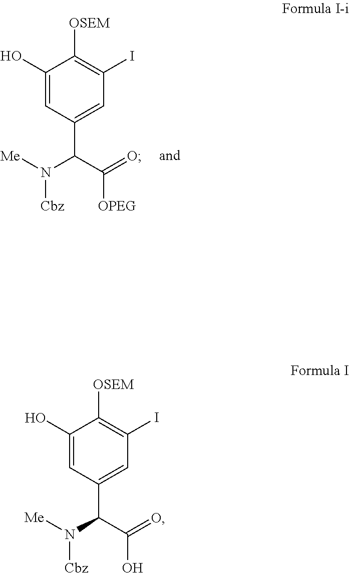

or a salt or solvate thereof; or,

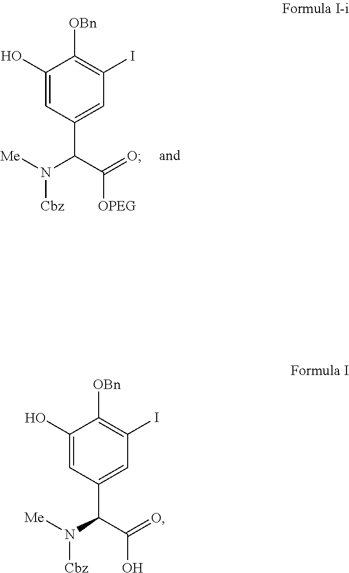

or a salt or solvate thereof; or,

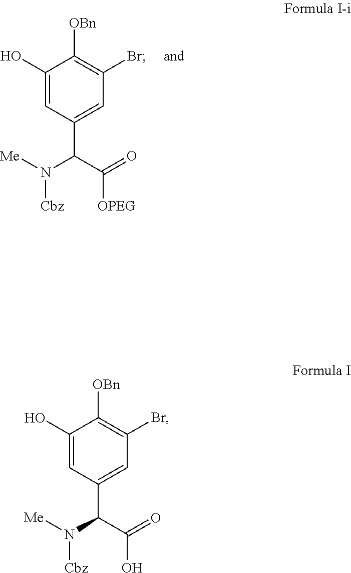

or a salt or solvate thereof; or,

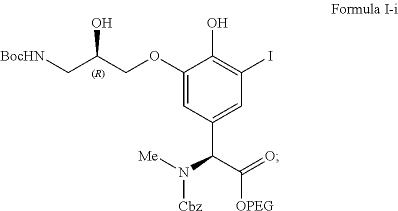

and Formula I is

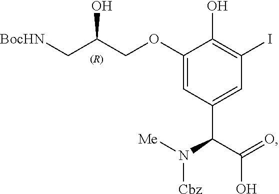

or a salt or solvate thereof; or,

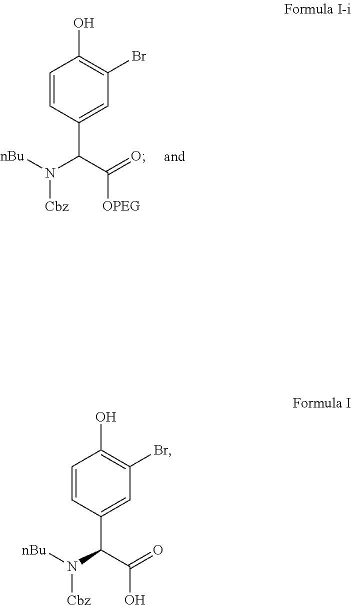

or a salt or solvate thereof; or,

### Formula I-i is

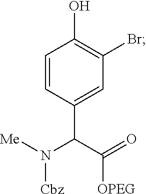

### and Formula I is

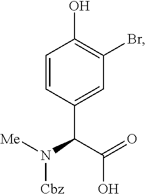 [SMILES: CN(C(=O)OCc1ccccc1)[C@H](C(=O)O)c1ccc(O)c(Br)c1]

or a salt or solvate thereof; or,

### Formula I-i is

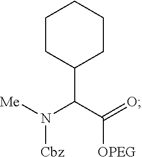

### and Formula I is

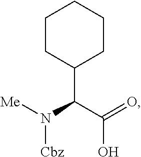 [SMILES: CN(C(=O)OCc1ccccc1)[C@H](C(=O)O)C1CCCCC1]

or a salt or solvate thereof; or,

### Formula I-i is

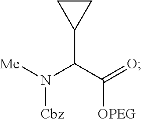

### and Formula I is

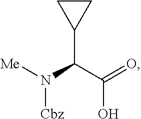 [SMILES: CN(C(=O)OCc1ccccc1)[C@H](C(=O)O)C1CC1]

or a salt or solvate thereof; or,

### Formula I-i is

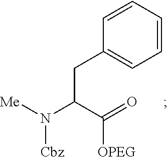

### and Formula I is

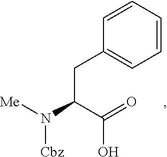 [SMILES: CN(C(=O)OCc1ccccc1)[C@@H](Cc1ccccc1)C(=O)O]

or a salt or solvate thereof; or,

### Formula I-i is

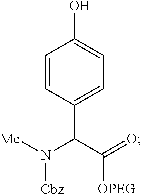

### and Formula I is

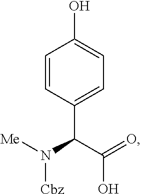 [SMILES: CN(C(=O)OCc1ccccc1)[C@H](C(=O)O)c1ccc(O)cc1]

or a salt or solvate thereof; or,

### Formula I-i is

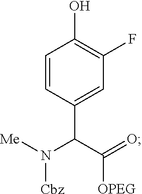

### and Formula I is

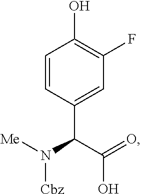 [SMILES: CN(C(=O)OCc1ccccc1)[C@H](C(=O)O)c1ccc(O)c(F)c1]

or a salt or solvate thereof; or,

### Formula I-i is

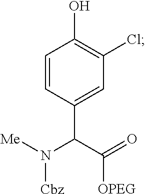

### and Formula I is

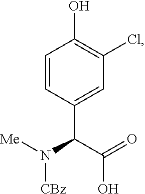

or a salt or solvate thereof; or,

### Formula I-i is

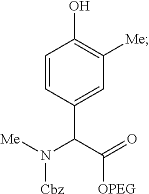

### and Formula I is

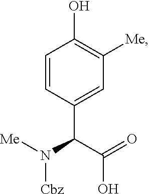 [SMILES: Cc1cc([C@@H](C(=O)O)N(C)C(=O)OCc2ccccc2)ccc1O]

or a salt or solvate thereof; or,

### Formula I-i is

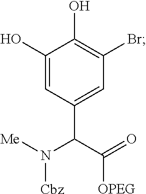

### and Formula I is

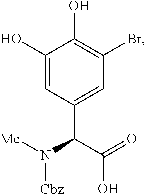 [SMILES: CN(C(=O)OCc1ccccc1)[C@H](C(=O)O)c1cc(O)c(O)c(Br)c1]

or a salt or solvate thereof; or,

### Formula I-i is

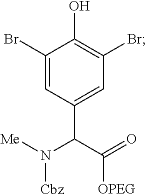

### and Formula I is

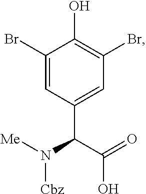 [SMILES: CN(C(=O)OCc1ccccc1)[C@H](C(=O)O)c1cc(Br)c(O)c(Br)c1]

or a salt or solvate thereof; or,

### Formula I-i is

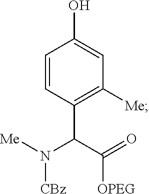

### and Formula I is

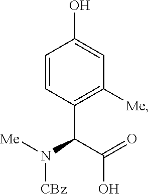

or a salt or solvate thereof; or,

### Formula I-i is

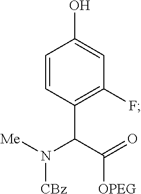

### and Formula I is

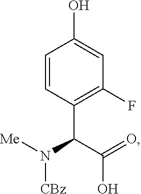

or a salt or solvate thereof; or,

### Formula I-i is

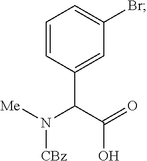

### and Formula I is

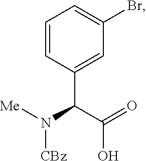

or a salt or solvate thereof; or,

### Formula I-i is

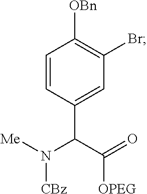

### and Formula I is

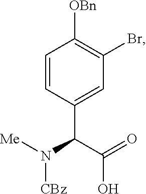

or a salt or solvate thereof; or,

### Formula I-i is

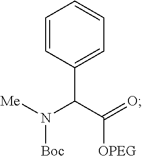

### and Formula I is

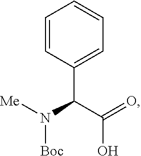 [SMILES: CN(C(=O)OC(C)(C)C)[C@H](C(=O)O)c1ccccc1]

or a salt or solvate thereof;
wherein, in each instance, PEG is —(CH₂CH₂—O)_{q}-Me, wherein q is 1, 2, or 3.

Embodiment II-85. The method of Embodiment II-84, wherein the enantiomeric mixture of Formula I-i and the compound of Formula I, or a salt or solvate thereof, are:

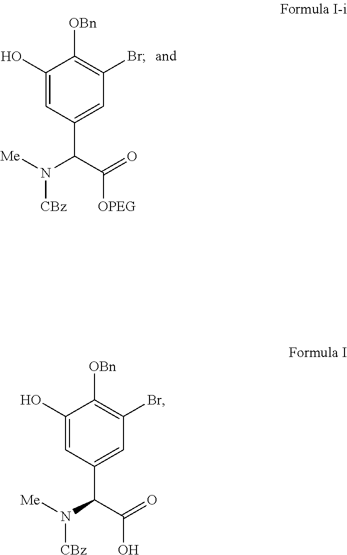

or a salt or solvate thereof;
wherein PEG is —(CH₂CH₂—O)_{q}—CH₃, wherein q is 1, 2, or 3.

Embodiment II-86. The method of Embodiment II-1, wherein the enantiomeric mixture of Formula I-i and the compound of Formula I, or a salt or solvate thereof, are:

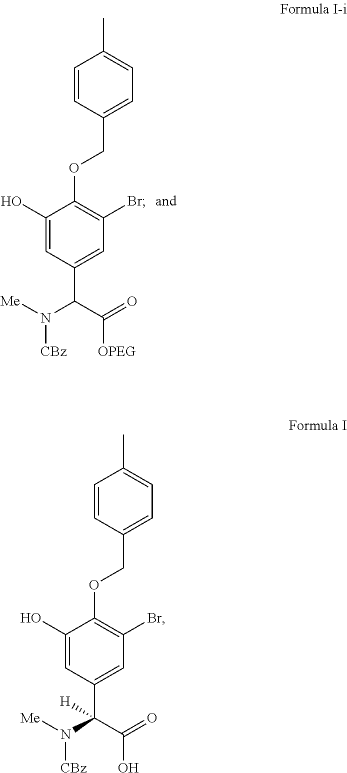

or a salt or solvate thereof;
wherein PEG is —(CH₂CH₂—O)_{q}—CH₃, wherein q is 1, 2, or 3.

Embodiment II-87. The method of Embodiment II-1, wherein the enantiomeric mixture of Formula I-i and the compound of Formula I, or a salt or solvate thereof, are:

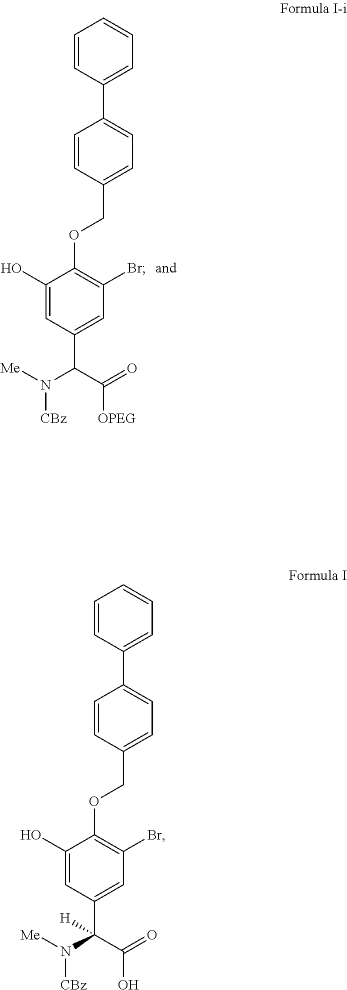

or a salt or solvate thereof;
wherein PEG is —(CH₂CH₂—O)_{q}—CH₃, wherein q is 1, 2, or 3.

Embodiment II-88. The method of any one of Embodiments II-84 to II-87, wherein q is 1.

Embodiment II-89. The method of any one of Embodiments II-84 to II-87, wherein q is 2.

Embodiment II-90. The method of any one of Embodiments II-84 to II-87, wherein q is 3.

Embodiment II-91. The method of Embodiment II-1, wherein the enantiomeric mixture of Formula I-i and the compound of Formula I, or a salt or solvate thereof, are:

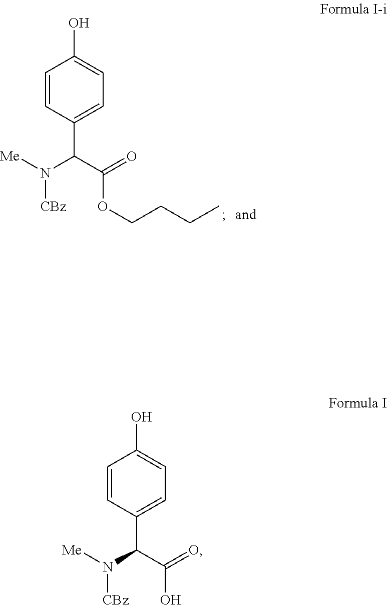

or a salt or solvate thereof; or,

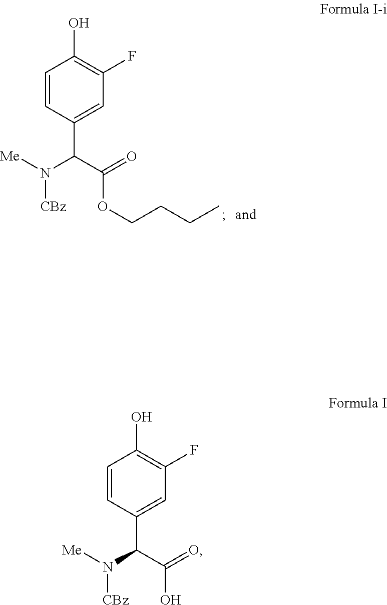

or a salt or solvate thereof; or,

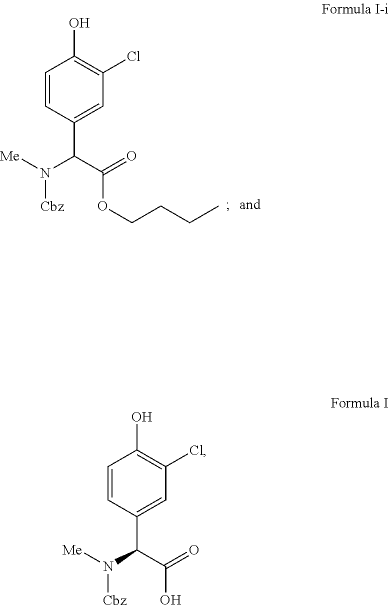 [SMILES: CCCCOC(=O)C(c1ccc(O)c(Cl)c1)N(C)C(=O)OCc1ccccc1 ; CN(C(=O)OCc1ccccc1)[C@H](C(=O)O)c1ccc(O)c(Cl)c1]

or a salt or solvate thereof; or,

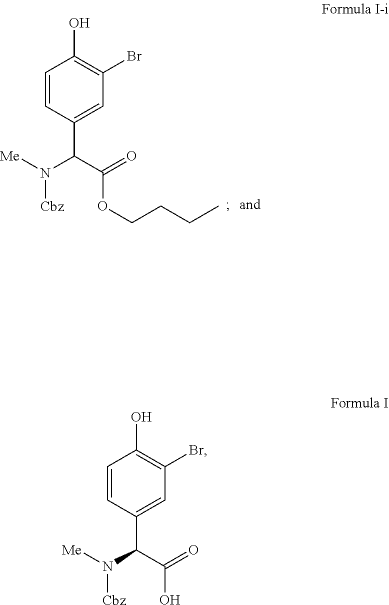 [SMILES: CCCCOC(=O)C(c1ccc(O)c(Br)c1)N(C)C(=O)OCc1ccccc1 ; CN(C(=O)OCc1ccccc1)[C@H](C(=O)O)c1ccc(O)c(Br)c1]

or a salt or solvate thereof; or,

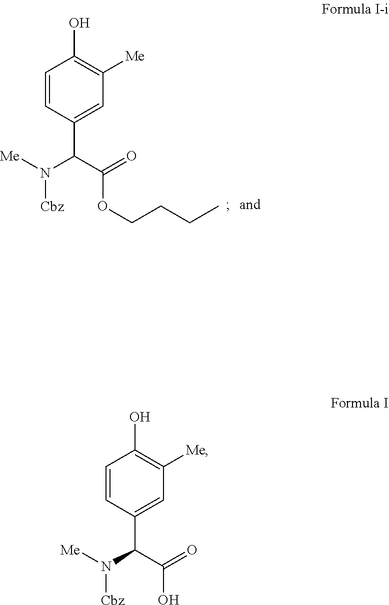 [SMILES: CCCCOC(=O)C(c1ccc(O)c(C)c1)N(C)C(=O)OCc1ccccc1 ; Cc1cc([C@@H](C(=O)O)N(C)C(=O)OCc2ccccc2)ccc1O]

or a salt or solvate thereof; or,

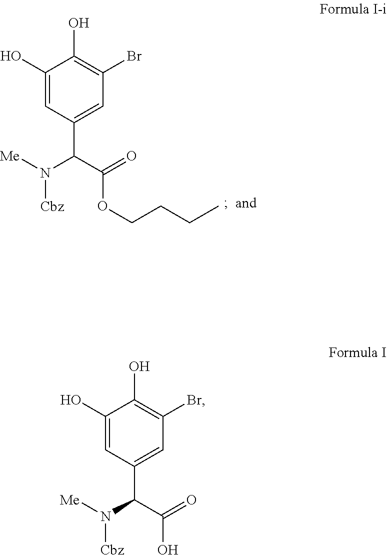 [SMILES: CCCCOC(=O)C(c1cc(O)c(O)c(Br)c1)N(C)C(=O)OCc1ccccc1 ; CN(C(=O)OCc1ccccc1)[C@H](C(=O)O)c1cc(O)c(O)c(Br)c1]

or a salt or solvate thereof; or,

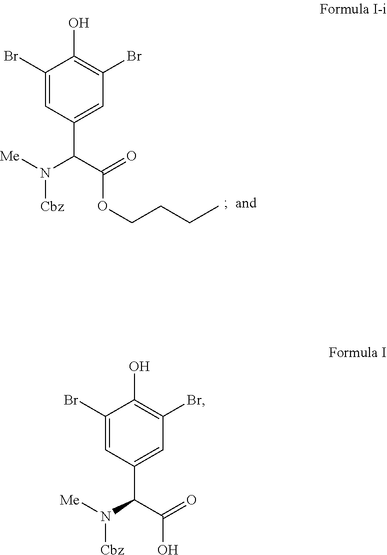 [SMILES: CCCCOC(=O)C(c1cc(Br)c(O)c(Br)c1)N(C)C(=O)OCc1ccccc1 ; CN(C(=O)OCc1ccccc1)[C@H](C(=O)O)c1cc(Br)c(O)c(Br)c1]

or a salt or solvate thereof; or,

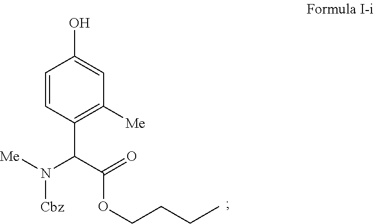 [SMILES: CCCCOC(=O)C(c1ccc(O)cc1C)N(C)C(=O)OCc1ccccc1]

### and Formula I is

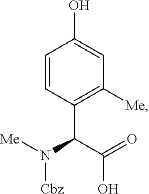 [SMILES: Cc1cc(O)ccc1[C@@H](C(=O)O)N(C)C(=O)OCc1ccccc1]

or a salt or solvate thereof; or,

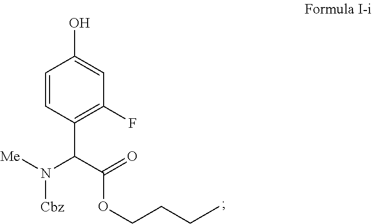 [SMILES: CCCCOC(=O)C(c1ccc(O)cc1F)N(C)C(=O)OCc1ccccc1]

### and Formula I is

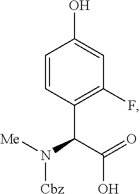 [SMILES: CN(C(=O)OCc1ccccc1)[C@H](C(=O)O)c1ccc(O)cc1F]

or a salt or solvate thereof; or,

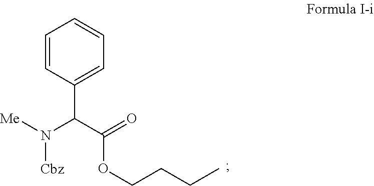 [SMILES: CCCCOC(=O)C(c1ccccc1)N(C)C(=O)OCc1ccccc1]

### and Formula I is

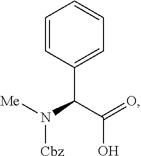 [SMILES: CN(C(=O)OCc1ccccc1)[C@H](C(=O)O)c1ccccc1]

or a salt or solvate thereof; or,

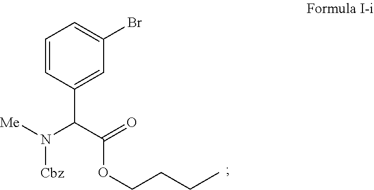 [SMILES: CCCCOC(=O)C(c1cccc(Br)c1)N(C)C(=O)OCc1ccccc1]

### and Formula I is

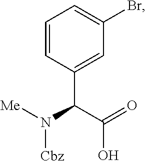 [SMILES: CN(C(=O)OCc1ccccc1)[C@H](C(=O)O)c1cccc(Br)c1]

or a salt or solvate thereof; or,

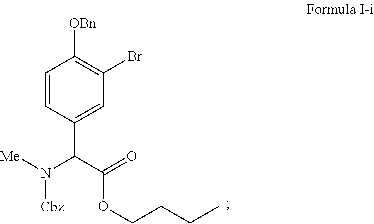 [SMILES: CCCCOC(=O)C(c1ccc(OCc2ccccc2)c(Br)c1)N(C)C(=O)OCc1ccccc1]

### and Formula I is

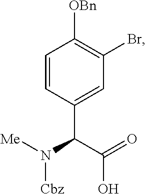 [SMILES: CN(C(=O)OCc1ccccc1)[C@H](C(=O)O)c1ccc(OCc2ccccc2)c(Br)c1]

or a salt or solvate thereof.

Embodiment II-92. The method of Embodiment II-1, wherein the enantiomerically enriched compound of Formula I is:

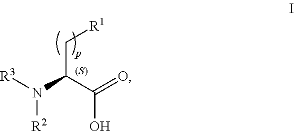

or a salt or solvate thereof,
wherein,
   R¹ is selected from the group consisting of: C₃₋₆ cycloalkyl and phenyl, wherein each can be unsubstituted or substituted with one, two, three, or four substituents independently selected from the group consisting of hydroxyl, fluoro, chloro, bromo, and methyl;
   R² is a carbamate N-protecting group;
   R³ is methyl; and
   p is 1 or zero;
the method comprising:
   contacting in an aqueous solution, wherein the aqueous solution comprises PEG400, at a temperature of about 45° C., an enantiomeric mixture of Formula I-i:

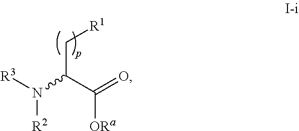

wherein,
   R¹, R², R³, and p are as defined in the compound of Formula I; and
   R^{a} is n-butyl or —(CH₂CH₂O)_{q}-Me, wherein q is 2 or 3;
with a solution of Lipase 147, Evoxx Technologies GmbH, evo-1.3.147.S dissolved in aqueous glycine buffer at a pH from about 9.0 to about 10.0, to produce the enantiomerically enriched compound of Formula I, or a salt or solvate thereof.

Embodiment II-93. The method of Embodiment II-92, wherein R^{a} is —(CH₂CH₂O)_{q}-Me.

Embodiment II-94. The method of Embodiment II-93, wherein q is 2.

Embodiment II-95. The method of Embodiment II-93, wherein q is 3.

Embodiment II-96. The method of Embodiment II-92, wherein R^{a} is n-butyl.

Embodiment II-97. The method of any one of Embodiments II-92 to II-96, wherein R² is Cbz.

Embodiment II-98. The method of any one of Embodiments II-92 to II-97, wherein R¹ is unsubstituted or substituted C₃₋₆ cycloalkyl.

Embodiment II-99. The method of any one of Embodiments II-92 to II-97, wherein R¹ is unsubstituted or substituted phenyl.

Embodiment II-100. The method of any one of Embodiments II-1 to II-99, wherein the contacting is under dynamic kinetic resolution conditions.

Embodiment II-101. The method of any one of Embodiments II-1 to II-100, further comprising:
a) adding a first organic solvent to an aqueous solution comprising the produced enantiomerically enriched compound of Formula I to form a first mixture;
b) separating the first mixture into a first aqueous phase and a first organic phase;
c) adjusting the pH of the first aqueous phase to about 7.0;
d) adding a second organic solvent to the first aqueous phase to form a second mixture;
e) adjusting the pH of the second mixture to about 2.0 to about 3.0;
f) separating the second mixture into a second aqueous phase and second organic phase; and
g) concentrating the second organic phase,
   to prepare the enantiomerically enriched compound of Formula I, or a salt or solvate thereof.

Embodiment II-102. The method of any one of Embodiments II-1 to II-100, further comprising:
a) adding a first organic solvent to an aqueous solution comprising the produced enantiomerically enriched compound of Formula I to form a first mixture;
b) separating the first mixture into a first aqueous phase and a first organic phase;
c) adding a second organic solvent to the first aqueous phase to form a second mixture;
d) adjusting the pH of the second mixture to below about 7.0;
e) separating the second mixture into a second aqueous phase and second organic phase; and
f) concentrating the second organic phase,
   to prepare the enantiomerically enriched compound of Formula I, or a salt or solvate thereof.

Embodiment II-103. The method of Embodiment II-101 or II-102, wherein the first organic solvent is isopropyl acetate, cyclohexanes, or MTBE, or a combination of any of the foregoing.

Embodiment II-104. The method of any one of Embodiments II-101 to II-103, wherein the second organic solvent is isopropyl acetate, cyclohexanes, or MTBE, or a combination of any of the foregoing.

Embodiment II-105. The method of any one of Embodiments II-101 to II-104, wherein step a) further comprises adding water to the aqueous solution.

Embodiment II-106. The method of any one of Embodiments II-1 to II-105, further comprising isolating the enantiomerically enriched compound of Formula I, or a salt or solvate thereof.

Embodiment II-107. The method of any one of Embodiments II-1 to II-106, wherein the enantiomerically enriched compound of Formula I is a salt.

Embodiment II-108. The method of Embodiment II-107, wherein the salt of the enantiomerically enriched compound of Formula I is a lysine, piperazine, or dicyclohexylamine salt.

Embodiment II-109. The method of Embodiment II-107 or II-108, wherein the salt of the enantiomerically enriched compound of Formula I is crystalline.

Embodiment II-110. A lysine salt of compound 2ac:

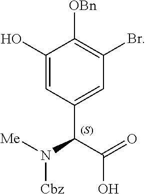

Embodiment II-111. The lysine salt of Embodiment II-110, wherein the lysine salt is crystalline.

Embodiment II-112. The lysine salt of Embodiment II-110 or II-111, characterized by an XRPD pattern comprising at least five peaks selected from the group consisting of 3.8, 6.9, 13.3, 13.9, 20.9, 22.0, 22.4, and 25.2 °2θ±0.1 °2θ.

Embodiment II-113. The lysine salt of any one of Embodiments II-110 to II-112, characterized by an XRPD pattern substantially the same as the pattern shown in FIG. 1.

Embodiment II-114. The lysine salt of any one of Embodiments II-110 to II-113, characterized by a thermogravimetric (TG) thermogram indicating continuous weight loss of about 4.09% from between about 18.9° C. to about 150° C.

Embodiment II-115. The lysine salt of any one of Embodiments II-110 to II-114, characterized by a TG thermogram substantially the same as the pattern shown in FIG. 2.

Embodiment II-116. The lysine salt of any one of Embodiments II-110 to II-115, characterized by a differential scanning calorimetry (DSC) thermogram comprising endothermic peak between about 133° C. and about 137° C. and an endothermic peak between about 192° C. and about 196° C.

Embodiment II-117. The lysine salt of any one of Embodiments II-110 to II-116, characterized by a DSC thermogram comprising an endothermic peak at about 134.6° C. and an endothermic peak at about 194.4° C.

Embodiment II-118. The lysine salt of any one of Embodiments II-110 to II-117, characterized by a DSC thermogram substantially the same as the pattern shown in FIG. 2.

Embodiment II-119. The lysine salt of any one of Embodiments II-110 to II-118 prepared by the method of any one of Embodiment II-1, 84, 85, or 92.

Embodiment II-120. A piperazine salt of compound 2ac:

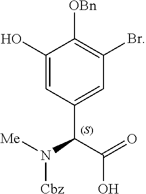

Embodiment II-121. The piperazine salt of Embodiment II-120, wherein the piperazine salt is crystalline.

Embodiment II-122. The piperazine salt of Embodiment II-120 or II-121, characterized by an XRPD pattern comprising at least two peaks selected from the group consisting of 11.0, 12.6, and 19.0 °2θ±0.1 °2θ.

Embodiment II-123. The piperazine salt of any one of Embodiments II-120 to II-122, characterized by an XRPD pattern substantially the same as the pattern shown in FIG. 3.

Embodiment II-124. The piperazine salt of any one of Embodiments II-120 to II-123, characterized by a thermogravimetric (TG) thermogram indicating continuous weight loss of about 4.93% from between about 27.6° C. to about 100° C.

Embodiment II-125. The piperazine salt of any one of Embodiments II-120 to II-124, characterized by a TG thermogram substantially the same as the pattern shown in FIG. 4.

Embodiment II-126. The piperazine salt of any one of Embodiments II-120 to II-125, characterized by a differential scanning calorimetry (DSC) thermogram comprising an endothermic peak between about 88° C. and about 92° C.

Embodiment II-127. The piperazine salt of any one of Embodiments II-120 to II-126, characterized by a DSC thermogram comprising an endothermic peak at about 89.8° C.

Embodiment II-128. The piperazine salt of any one of Embodiments II-120 to II-127, characterized by a DSC thermogram substantially the same as the pattern shown in FIG. 4.

Embodiment II-129. The piperazine salt of any one of Embodiments II-120 to II-128 prepared by the method of any one of Embodiments II-1, II-84, II-85, or II-92.

Embodiment II-130. A dicyclohexylamine salt of compound 2ac:

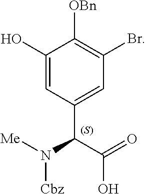

Embodiment II-131. The dicyclohexylamine salt of Embodiment II-130, wherein the dicyclohexylamine salt is crystalline.

Embodiment II-132. The dicyclohexylamine salt of Embodiment II-130 or II-131, characterized by an XRPD pattern comprising at least five peaks selected from the group consisting of 5.2, 7.5, 10.3, 13.7, 15.0, 16.2, 17.6, 20.5, 22.4, and 38.1 °20±0.1 °2θ.

Embodiment II-133. The dicyclohexylamine salt of any one of Embodiments II-130 to II-132, characterized by an XRPD pattern substantially the same as the pattern shown in FIG. 5.

Embodiment II-134. The dicyclohexylamine salt of any one of Embodiments II-130 to II-133, characterized by a thermogravimetric (TG) thermogram indicating continuous weight loss of about 3.58% between about 25° C. and about 130° C.

Embodiment II-135. The dicyclohexylamine salt of any one of Embodiments II-130 to II-134, characterized by a TG thermogram substantially the same as the pattern shown in FIG. 6.

Embodiment II-136. The dicyclohexylamine salt of any one of Embodiments II-130 to II-135, characterized by a differential scanning calorimetry (DSC) thermogram comprising an endothermic peak between about 175° C. and about 179° C.

Embodiment II-137. The dicyclohexylamine salt of any one of Embodiments II-130 to II-136, characterized by a DSC thermogram comprising an endothermic peak at about 177.01° C.

Embodiment II-138. The dicyclohexylamine salt of any one of Embodiments II-130 to II-137, characterized by a DSC thermogram substantially the same as the pattern shown in FIG. 6.

Embodiment II-139. The dicyclohexylamine salt of any one of Embodiments II-130 to II-138 prepared by the method of any one of Embodiments II-1, II-84, II-85, or II-92.

Embodiment II-140. A dicyclohexylamine salt of compound 2ag:

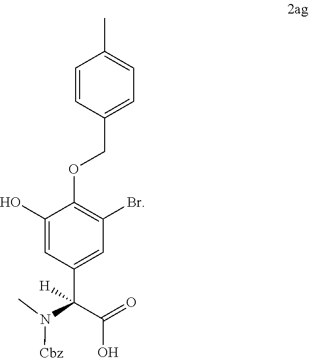 [SMILES: Cc1ccc(COc2c(O)cc([C@@H](C(=O)O)N(C)C(=O)OCc3ccccc3)cc2Br)cc1]

Embodiment II-141. The dicyclohexylamine salt of Embodiment II-140, wherein the dicyclohexylamine salt is crystalline.

Embodiment II-142. The dicyclohexylamine salt of Embodiment II-140 or II-141 prepared by the method of any one of Embodiments II-1, II-86, or II-92.

Embodiment II-143. A dicyclohexylamine salt of compound 2ah:

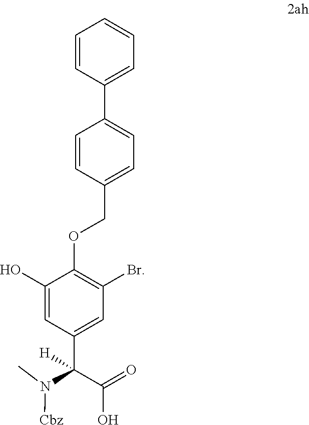 [SMILES: CN(C(=O)OCc1ccccc1)[C@H](C(=O)O)c1cc(O)c(OCc2ccc(-c3ccccc3)cc2)c(Br)c1]

Embodiment II-144. The dicyclohexylamine salt of Embodiment II-143, wherein the dicyclohexylamine salt is crystalline.

Embodiment II-145. The dicyclohexylamine salt of Embodiment II-143 or II-144 prepared by the method of any one of Embodiments II-1, II-87, or II-92.

### EXAMPLES

The Examples provide exemplary methods for preparing compounds. The skilled artisan will appreciate that other synthetic routes may be used to synthesize the compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to the skilled artisan.

### Example 1—Enzyme Screening

A preliminary microscale screen for carboxylic ester hydrolase activity was performed with a library containing 277 hydrolytic enzymes (lipases, esterases, proteases and acylases) on racemic N-methyl-hydroxyphenylglycine analog 1a. Treating an iPrOH solution of 1a with 100 wt % of each enzyme in a 7.5 mM Tris-buffered aqueous media at pH 8 for 2 days at room temperature gave six hits with significant HPLC conversions (9-51 A % HPLC) and high enantiomeric excess (Table 1). In subsequent validation experiments, 1a (2.5 mg, 5.9 μmol) in 50 μL of iPrOH was treated with 200 wt % of each enzyme (in order to ensure detection of activity, ~5 mg enzyme was added) in a 10 mM Tris-buffered aqueous media at pH 8 (950 μL) and the resulting mixture was stirred at room temperature for 2 days. Commercially available carboxylic ester hydrolase Lipase 147, Evoxx Technologies GmbH, Enzyme identifier: evo-1.3.147.S, gave the highest conversion of product 2a with the desired (S) selectivity (Table 1, entry 6).

Scheme 2 depicts the synthetic routes in Example 1.

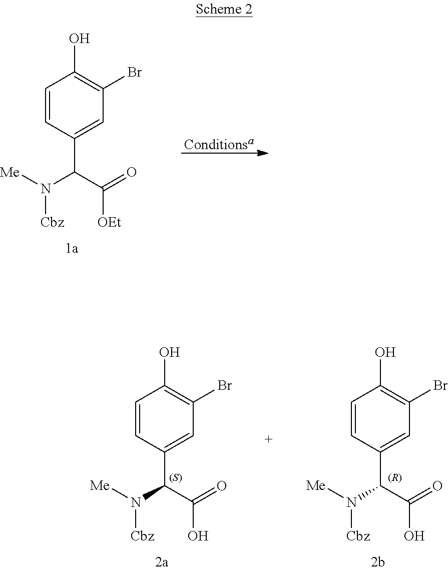

TABLE 1
Reaction Results and Data.

| | | Enzyme | Convn | ee | Product | |
|---|---|---|---|---|---|---|
| Entry | Supplier | Identifier | (%) | (%) | Selectivity | Product |
| 1 | Fluka | 94734 | 9 | 99.7 | R | 2b |
| 2 | Sorachim | COE-313 | 15 | 99.9 | R | 2b |
| 3 | Toyobo | LIP-301; | 19 | 99.9 | R | 2b |
| | | LPL-311 | | | | |
| 4 | Sorachim | COE-311 | 21 | 99.9 | R | 2b |
| 5 | Amano | CES-E2 | 24 | 99.8 | S | 2a |
| 6 | EVOXX | evo-1.3.147.S | 51 | 94.2 | S | 2a |

Conversions were determined by using the measured enantiomeric excess of the substrates (esters) and products (acids) according to the method described by Chen, C. S.; Fujimoto, Y.; Girdaukas, G; Sih, C. J. Quantitative Analyses of Biochemical Kinetic Resolutions of Enantiomers J. Am. Chem. Soc. 1982, 104, 7294-7299.

### Example 2: Determination of DKR Conditions

The potential for a dynamic kinetic resolution was evaluated. In the absence of any carboxylic ester hydrolase (or lipase), it was found that the experimental range for a dynamic kinetic resolution that would minimize the background hydrolysis reaction was between 35-45° C. and pH 8-9.5 (pH maintained by high buffer loading, high buffer capacity, or static pH). Conditions in agreement with the Hoffmann test were used to show that DKR conditions had been reached. Hirsch, R.; Hoffmann, R. W. Chiral Organometallic Reagents, V[1] A Test on the Configurational Stability of Chiral Organolithium Compounds Based on Kinetic Resolution; Scope and Limitations Chem. Ber. 1995, 125, 975-982.

A variety of N-methyl-hydroxyphenylglycine esters 1 were synthesized and their relative enzymatic hydrolysis activity with Lipase 147, Evoxx Technologies GmbH, evo-1.3.147.S was explored (Table 2a, entries 1-6). The stability to chemical hydrolysis of the esters was also tested for a 20 hour incubation period in different buffers (Table 2b).

In view of these results, dynamic kinetic resolution conditions were modified using n-butyl ester 1c due to its good enzymatic activity and high chemical stability to hydrolysis, as well as PEGylated ester 1f due to its high enzymatic activity.

Scheme 3a depicts synthetic routes used in Example 2 to assess the relative enzymatic hydrolysis activity

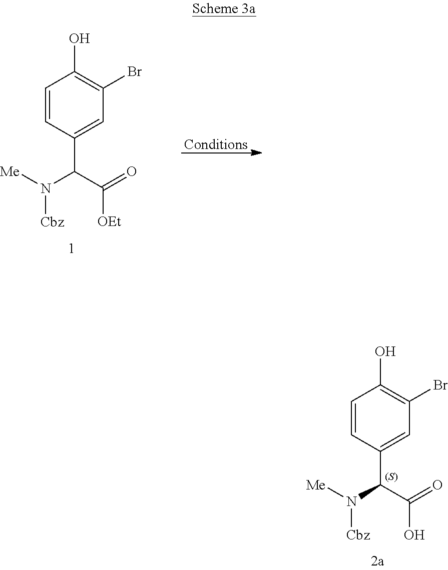

TABLE 2a
Reaction Results, Data and Conditions.

| | | | Relative | | | | | | | |
|---|---|---|---|---|---|---|---|---|---|---|
| | | | Enzymatic | | Lipase | | | | | |
| | | | Activity | Substrate 1 | L111 | | Initial | T | Reaction | Conv. |
| Entry | 1 | R | (%) | (mg) | (mg) | Buffer (mL) and additive | pH | (° C.) | Time (h) | (%) |
| 1 | 1b | Me | 72 | 5 | 10 | 0.1M Glycine (1 mL) | 9 | 56 | 17 | 46 |
| 2 | 1a | Et | 100 | 5 | 10 | 0.1M Glycine (1 mL) | 9 | 56 | 17 | 63 |
| | | | | 5 | 5 | 0.1M TRIS (1 mL), 10% PEG200 | 8.7 | 45 | 20 | 54 |
| 3 | 1c | n-Bu | 75 | 20 | 2 | 1.0M Glycine (0.4 mL), 10% PEG400 | 10.2 | 45 | 44 | 99 |
| | | | | 5 | 5 | 0.1M TRIS (1 mL), 10% PEG200 | 8.7 | 45 | 20 | 41 |
| | | | | 5 | 5 | 1.0M Glycine (1 mL) | 9.8 | 45 | 19 | 55 |
| 4 | 1d | n-Hex | 32 | 5 | 5 | 1.0M Glycine (1 mL) | 9.8 | 45 | 19 | 24 |
| 5 | 1e | n-Hept | 16 | 5 | 5 | 1.0M Glycine (1 mL) | 9.8 | 45 | 19 | 12 |
| 6 | 1f | (CH₂CH₂O)₃Me | >250 | 1250 | 25 | 1.0M Glycine (25 mL), 10% PEG400 | 9.2 | 45 | 44 | 100 |

[a] General Conditions: To a solution of lipase L111 in buffer an additive, was added substrate 1. The reaction was allowed to proceed at defined temperature, pH and time. Conversions were measured by HPLC.

Scheme 3b depicts synthetic routes used in Example 2 to assess the stability of the esters to chemical hydrolysis

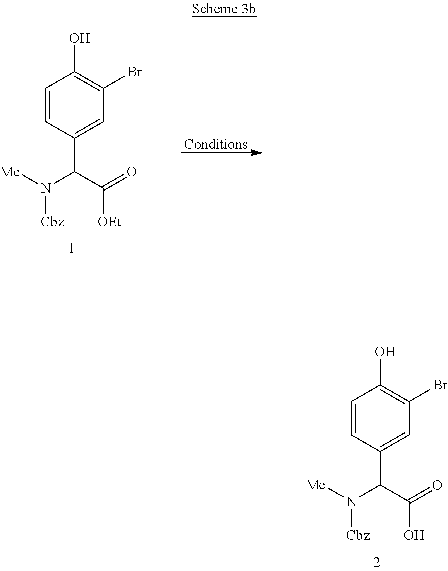 [SMILES: CCOC(=O)C(c1ccc(O)c(Br)c1)N(C)C(=O)OCc1ccccc1 ; "Conditions" ; CN(C(=O)OCc1ccccc1)C(C(=O)O)c1ccc(O)c(Br)c1]

TABLE 2b
Reaction Results, Data and Conditions.

| | | | Chemical | | | | | |
|---|---|---|---|---|---|---|---|---|
| | | | Hydrolysis | Substrate 1 | | | T | Reaction |
| Entry | 1 | R | (A %) | (mg) | Buffer (mL) and additive | Initial pH | (° C.) | Time (h) |
| 1 | 1b | Me | 1.7 | 5 | 0.1M TRIS (1 mL) | 8.8 | 45 | 20 |
| 2 | 1a | Et | 0.8 | 5 | 0.1M TRIS (1 mL) | 8.8 | 45 | 60 |
| 3 | 1c | n-Bu | 0 | 5 | 0.1M TRIS (1 mL) | 9.2 | 50 | 20 |
| 4 | 1f | (CH₂CH₂O)₃Me | 2.1 | 5 | 1.0M Glycine (1 mL) | 8.8 | 45 | 20 |

[a] General conditions: Substrate 1 was incubated in the absence of lipase L111 at defined buffer, pH, temperature and reaction time. Chemical hydrolysis is measure by HPLC.

### Example 3: Esters and Enzyme Loading Screen

Reaction conditions were modified, such as temperature (reactivity), buffers (pH), salts (salting in and out effects), solvents (substrate accessibility) and bases (racemization rate). 1 M aqueous glycine buffer, pH 9.6, at 45° C. and the addition of 10%-20% v/v of polyethyleneglycol (PEG200 or PEG400) provided improvements in the conversions and eventually allowed for reduction of the carboxylic ester hydrolase (or lipase) loading from 100 wt % to much lower loadings (Table 3). Additional bases and salts had no beneficial impact.

The observed solubilizing properties of the polyethyleneglycol (PEG200 or PEG400) as co-solvent led to design and synthesis of new PEGylated N-alkyl amino acid derivatives, integrating the hydrophilic PEG chains directly onto the substrates (Table 3, entries 8-10). The PEGylated (1f and 1h) N-alkyl amino acid esters exhibited improved aqueous solubility and enzymatic reactivity, which allowed for reduction of the enzyme loading to 5 wt % while maintaining >95% conversion and >98% ee (Table 3, entries 8-10).

Scheme 4 depicts the synthetic routes in Example 3.

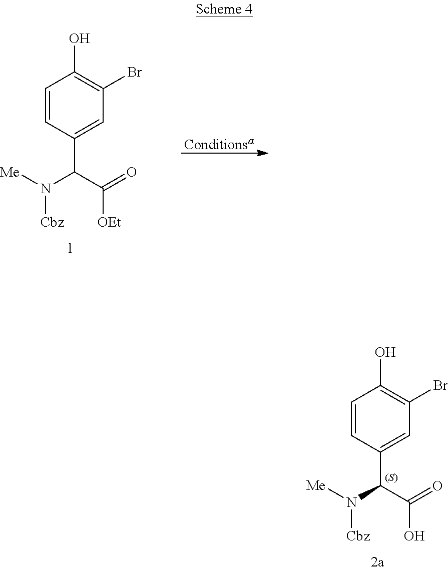

TABLE 3
Reaction Results, Data and Conditions

| | | | 2a | | |
|---|---|---|---|---|---|
| | 1 | | Enzyme | Convn^{e} | |
| Entry | 1 | R | (wt %) | (%) | ee^{e} |
| 1^{b} | 1b | Me | 100 | 35 | 93.0^{f} |
| 2^{c} | 1a | Et | 100 | 51 | 94.2 |
| 3^{c} | 1a | Et | 9 | 55 | 98.4 |
| 4^{c} | 1g | n-Pr | 9 | 66 | 99.6 |
| 5^{c} | 1c | n-Bu | 9 | >95 | 99.6 |
| 6^{d} | 1d | n-Hex | 9 | 45 | n.d.^{g} |
| 7^{d} | 1e | n-Hept | 9 | 27 | n.d.^{g} |
| 8^{c} | 1h | —(CH₂CH₂O)₂Me | 9 | >95 | 98.8 |
| 9^{c} | 1f | —(CH₂CH₂O)₃Me | 9 | >95 | 98.8 |
| 10^{c} | 1f | —(CH₂CH₂O)₃Me | 5 | >95 | 98.8 |

^{a}General Conditions: To a solution of lipase evo-1.3.147.S in aqueous glycine buffer (pH adjusted to 9.0) at 45° C. were added PEG 200 (10% v/v) and the substrate (1). Reaction time 16 h.
^{b}Condition A: general conditions with 0.1M glycine buffer at 56° C. and reaction time 40 h.
^{c}0.5M glycine buffer.
^{d}1M glycine buffer.
^{e}Determined by HPLC.
^{f}1b contaminated with ~2.8 A % HPLC of acid 2a and has low chemical stability.
^{g}not determined

### Example 4: Verification on Gram Scale

Reactions were repeated on gram scale at 20 mL/g (5 w/v %, 1.25 g) substrate loading with 1c and 1f, where the pH of the reaction was adjusted to pH 9.2 (pH stat using 1 M sodium carbonate solution) for 1c and stayed roughly at pH 8.7 for 1f. Because of the acidity of the phenol component of the substrates, adjustment of the pH of the initial reaction medium was found to be important to ensure adequate substrate racemization, while still maintaining enzyme activity. Therefore, complete conversion and 99% ee of 2a from 1c was observed after 65 h in 1 M glycine buffer and 10% (v/v) PEG 200 when using 13.3 wt % of Lipase 147, Evoxx Technologies GmbH, evo-1.3.147.S at 45° C. and pH 9.2. Complete conversion and 98.8% ee of 2a from 1f was observed after 21 h in 1 M glycine buffer and 10% (v/v) PEG 400 when using 10 wt % of Lipase 147, Evoxx Technologies GmbH, evo-1.3.147.S at 45° C. and pH 8.7. It was possible to further reduce the enzyme loading to 2 wt % for 1f and still achieve full conversion and 97.9% ee after 44 h.

### Example 5: Variability of Substrate

To establish the general scope and applicability of the method, the modified reaction conditions were applied to a series of aromatic and aliphatic PEGylated N-alkyl amino acid esters and n-butyl N-alkyl amino acid esters (Tables 4a and 4b). This method was demonstrated on 0.5 mmol scale and proved to be effective for the dynamic kinetic resolution of a variety of substrates (Tables 4a and 4b).

As many of the unnatural N-alkyl amino acids prepared herein were unknown, the absolute stereochemistry for 2v was determined to be (S) by comparing the sign of the optical rotation with literature data and assuming the same sense of induction for the other substrate. This accorded with the fact that Lipase 147, Evoxx Technologies GmbH, evo-1.3.147.S has demonstrated S selectivity. The absolute stereochemistry of 2v was determined by comparison with literature, see Hanessian, S.; Babonneau, V.; Boyer, N.; Mannoury 1a Cour, C.; Millan, M. J.; De Nanteuil, G. Design and synthesis of potential dual NK1/NK3 receptor antagonists Bioorg. Med. Chem. Lett. 2014, 24, 510-514. (S)-(Benzyloxycarbonyl-methyl-amino)-phenyl-acetic acid: +112.7 (c 1, CHCl₃); Experimental +126.6 (c 0.923, CHCl₃).

This methodology converted a series of PEGylated and N-alkylated-hydroxyphenylglycine esters with neutral, electron withdrawing or donating groups (Table 4a, entries 1-8 and 11-20 and Table 4b, entries 2-3), simple PEGylated N-methyl-phenylglycine esters (Table 4a, entries 21-24) and finally O-benzyl (or substituted O-benzyl) or O-SEM protected aromatic PEGylated amino acid esters (Table 4a, entries 25-28 and Table 4b, entries 1 and 4-7) to their corresponding enantiomerically enriched N-methyl-hydroxyphenylglycine and N-methyl-phenylglycine derivatives in high yields and high ee. Additionally, longer chains on the nitrogen such as N-butyl-hydroxyphenylglycine were well tolerated by the enzyme, producing 88% yield in 97% ee of the desired product 21 (Table 4a, entries 9-10). This method also allowed for preparation of aliphatic enantiomerically enriched unnatural N-alkyl amino acids, such as the ones reported in Tables 5a and 5b below.

Scheme 5 depicts the synthetic routes for the compounds of Table 4a and Table 4b.

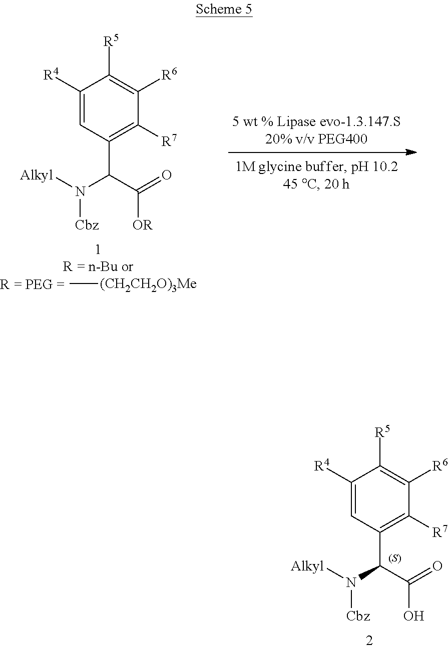

TABLE 4A
Reaction Results and Data.

| | 1 | Yield^{a} | ee^{b} | |
|---|---|---|---|---|
| Entry | R | (%) | (%) | 2 |
| 1 2 | PEG n-Bu | 84 69 | 98 96 | 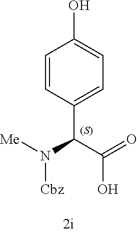 |
| 3 4 | PEG n-Bu | 98 92 | 98 99 | 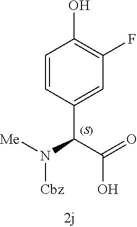 |
| 5 6 | PEG n-Bu | 75 78 | 97 99 | 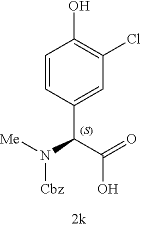 |
| 7 8 | PEG n-Bu | 91 84 | 98 99 | 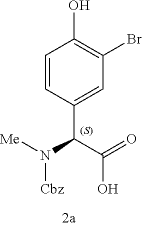 |
| 9 10 | PEG n-Bu | 88 n.a | 97 n.a | 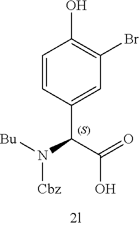 |
| 11 12 | PEG n-Bu | 67 65 | 97 99 | 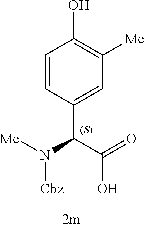 |
| 13 14 | PEG n-Bu | 83 16 | 92 n.a. | 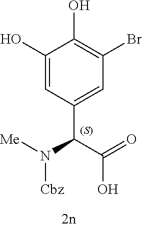 |
| 15 16 | PEG n-Bu | 87 2 | 93 n.a. | 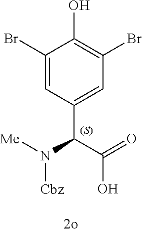 |
| 17 18 | PEG n-Bu | 62 12 | 85 n.a. | 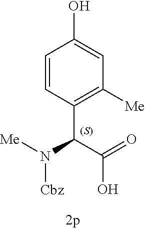 |
| 19 20 | PEG n-Bu | 77 33 | 96 98 | 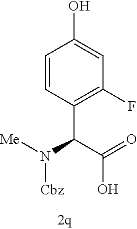 [SMILES: CN(C(=O)OCc1ccccc1)[C@H](C(=O)O)c1ccc(O)cc1F] |
| 21 22 | PEG n-Bu | 77 54 | 98 99 | 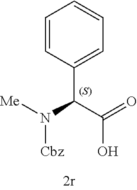 |
| 23 24 | PEG n-Bu | 74 41 | 89 91 | 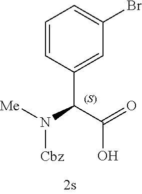 |
| 25 26 | PEG n-Bu | 89 17 | 98 98 | 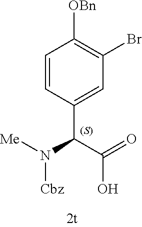 |
| 27 28 | PEG n-Bu | 97 n.a. | 97 n.a. | 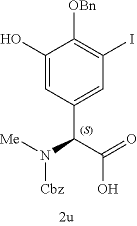 |

^{a}Isolated Yields.
^{b}Determined by HPLC analysis.
^{c}not available

TABLE 4b
Reaction Results and Data.

| | 1 | Conv^{b} | ee^{b} | |
|---|---|---|---|---|
| Entry | R | (A%) | (%) | 2 |
| 1 | PEG | 47 | 95 | 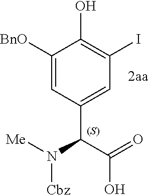 |
| 2 3 | PEG PEG | 25 97 | 99 92 | 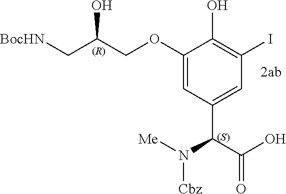 |
| 6 | PEG | 86 | 99 | 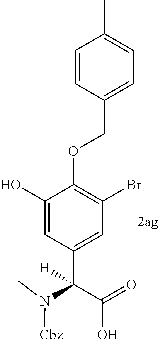 [SMILES: Cc1ccc(COc2c(O)cc([C@@H](C(=O)O)N(C)C(=O)OCc3ccccc3)cc2Br)cc1] |
| 4 | PEG | 85^{a} | 98 | 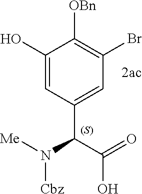 |
| 5 | PEG | 99 | 98 | 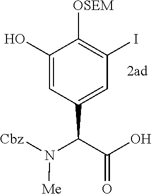 |
| 7 | PEG | 94 | 99 | 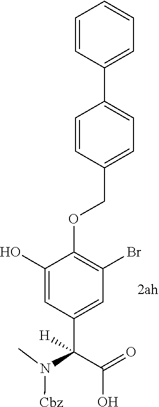 [SMILES: CN(C(=O)OCc1ccccc1)[C@H](C(=O)O)c1cc(O)c(OCc2ccc(-c3ccccc3)cc2)c(Br)c1] |

^{a}Isolated Yields.
^{b}Determined by HPLC analysis.

Scheme 6 depicts the synthetic routes for the compounds of Table 5a and Table 5b.

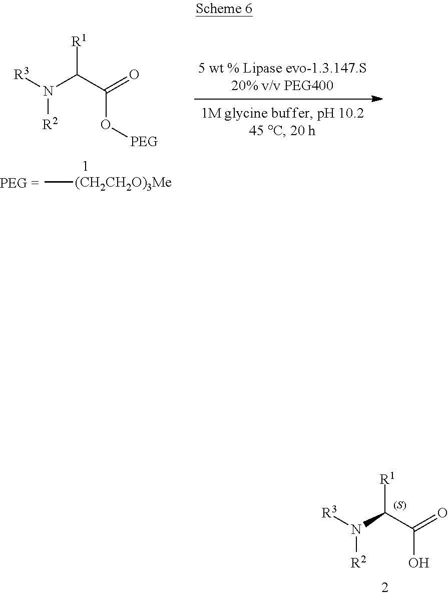

TABLE 5a
Reaction Results and Data.

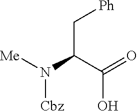 [SMILES: CN(C(=O)OCc1ccccc1)[C@@H](Cc1ccccc1)C(=O)O]

2v
74% yield, >95% ee^{a}

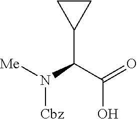 [SMILES: CN(C(=O)OCc1ccccc1)[C@H](C(=O)O)C1CC1]

2w
86 yield, 96% ee^{b}

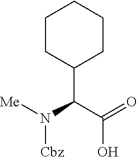 [SMILES: CN(C(=O)OCc1ccccc1)[C@H](C(=O)O)C1CCCCC1]

2x
41% yield, 81%ee^{b}

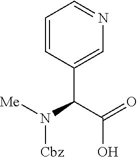 [SMILES: CN(C(=O)OCc1ccccc1)[C@H](C(=O)O)c1cccnc1]

2y
81% convn, 96% ee^{b}

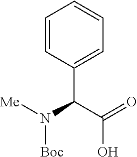 [SMILES: CN(C(=O)OC(C)(C)C)[C@H](C(=O)O)c1ccccc1]

2z
92% yield, 96% ee^{b}
^{a}Determined by comparing the optical rotation with literature.
^{b}Determined by HPLC.

TABLE 5b
Reaction Results and Data.

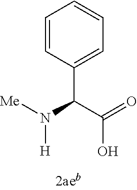 [SMILES: CN[C@H](C(=O)O)c1ccccc1]

100% conversion
13% ee^{a}
^{a}Determined by HPLC;

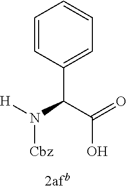 [SMILES: O=C(N[C@H](C(=O)O)c1ccccc1)OCc1ccccc1]

100% conversion
44% ee^{a}
R² may include H

### Example 6: Preparation of Enriched Amino Acid without Chromatographic Purification

This methodology detailed above allowed for the preparation of (S)-2-(((benzyloxy)carbonyl)(methyl)amino)-2-(3-bromo-4-hydroxyphenyl) acetic acid (2a) on a ~10 mmol scale. The methodology required low enzyme loading (5 wt %) and provided 2a in 94% yield and 98% ee without the need for chromatographic purification (eq 1). See Scheme 7.

To a 100 mL reaction vessel equipped with overhead stirring, temperature-controlled jacket, thermocouple, and pH probe, was added PEGylated amino acid ester 1f (5.0 g, 9.25 mmol, 1 equiv.) and PEG400 (7.5 mL, 1.5 mL/g) followed by 1 M aqueous glycine buffer (pH=9.6, 57.5 mL, 11.5 mL/g). Lipase 147, Evoxx Technologies GmbH, evo-1.3.147.S (250 mg, 5 wt %) was dissolved in 1 M aqueous glycine buffer (pH=9.6, 10 mL, 2 mL/g) by gentle agitation, then added to the reaction mixture. The reactor was heated to an internal temperature of 45° C. over 30 minutes and then stirred overnight. The reaction mixture was diluted with water (50 mL, 10 mL/g) and iPrOAc (100 mL, 20 mL/g), the layers were separated and the organic phase was discarded. Additional iPrOAc (100 mL, 20 mL/g) was added, the pH was adjusted to 4.0 with a solution of 20 wt % aqueous NaHSO₄ (11 mL) and the layers were separated. The organic layer was filtered through a 0.45 μM PTFE filter to remove residual precipitated protein, washed with 18 wt % NaCl (3×50 mL), and dried over Na₂SO₄, filtered and concentrated under vacuum to provide 2a (3.42 g, 94% yield, 98% ee). ¹H NMR (500 MHz, MeOD) δ: 7.47-7.23 (m, 6H), 7.15-7.10 (m, 1H), 6.87 (d, J=8.4 Hz, 1H), 5.75 (s (br), 1H), 5.17 (s (br), 2H), 2.75 (s, 3H); ¹³C NMR (126 MHz, MeOD) δ 175.5, 159.0, 153.7, 136.9, 133.8, 130.0, 129.6, 128.1, 127.5, 127.3, 115.6, 109.2, 67.0, 64.1, 30.5; HRMS HESI (m/z): [M+H]⁺ calcd for C₁₇H₁₇BrNO₅, 394.0290; found 394.0270. The enantiomeric excess was determined by chiral HPLC analysis, Tr (major) 5.54 min, Tr (minor) 5.88 min.

Scheme 7 depicts the synthetic route in Example 6.

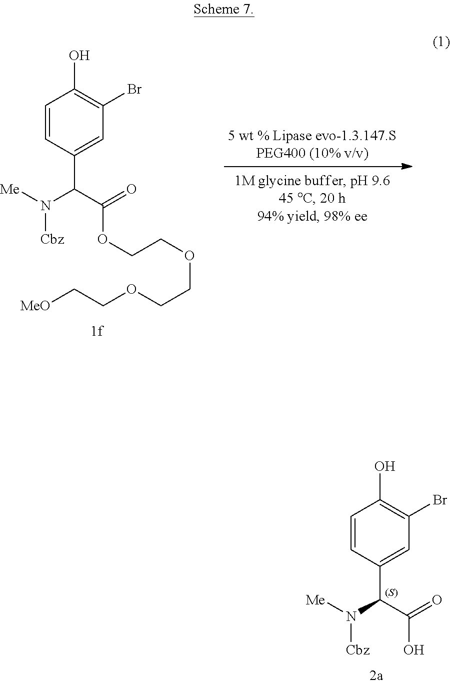

### Example 7: Optimized, Scaled Process of 2ac

After fine-tuning of the process conditions (pH 9.5 and 34° C.) for the dynamic kinetic resolution of the PEG-ester (Table 4b, entry 4), (2S)-2-(4-benzoxy-3-bromo-5-hydroxy-phenyl)-2-[carbobenzoxy(methyl)amino]acetic acid (2ac) was produced on ~2 mol scale in two batches.

### Process Description of a Single Batch:

A glycine buffer was prepared by mixing 10 L water and 244 g glycine and was pre-adjusted to pH 9.6 with 825 g of 8 w/v % sodium hydroxide solution (i.e., 8% NaOH weight in the sodium hydroxide solution). At 31° C. a substrate solution consisting of 622 g 2-(4-benzoxy-3-bromo-5-hydroxy-phenyl)-2-[carbobenzoxy(methyl)amino]acetic acid 2-[2-(2-methoxyethoxy) ethoxy]ethyl ester and 1.24 L 2-propanol was added into the glycine buffer. The pH dropped to 9.5 and the temperature increased to 34° C. by the addition of the substrate solution. The reaction started by the addition of 62 g (10 wt %) lipase 147, Evoxx Technologies GmbH, evo-1.3.147.S. The temperature was controlled to 34° C. and the pH was kept constant by the addition of 1 N NaOH until reaction completion (a total of 1.55 L in 25 h).

### Product Isolation Description of the Combined Two Batches:

The combined reaction solution was washed with an organic solution mixture consisting of 24 L cyclohexane and 1 L MTBE. The pH of separated water phase was adjusted to 7.0 by the addition of 542 mL 25 w/v % HCl (i.e., 25% HCl weight in the HCl solution). Subsequently 24 L of MTBE were added and the pH was further reduced to 2.5 by the addition of 720 mL 25 w/v % HCl and allowed to stir for another hour (complete precipitation of the lipase). The mixture was filtrated through a bed of 2 kg dicalite (lipase removal). The filter bed was washed twice with 5 L MTBE. The separated MTBE phase was washed twice with 20 L water.

As the product is a highly viscous gum, its MTBE solution was evaporated (40° C./450 mbar) and the remaining viscous oil re-dissolved in 10 L MTBE. The subsequent evaporation was proceeded until a final weight of the MTBE solution of 3166 g was achieved. 36.1 g of this solution were evaporated until 4 mbar were reached and kept for 8 hours. The remaining 11.1 g consisted of the target compound and remaining MTBE in a ratio of 1:0.28 molar (¹H NMR). The final product solution of 3166 g contained 925 g (96% yield) target compound (2ac) in a purity >99 area % and an ee of 99.0% (chiral HPLC).

Compounds 2ag and 2ah were scaled using the same methods provided for compound 2ac.

### Example 8: Production of Salts of 2ac, 2ag, and 2ah

### General Methods

### Ambient X-Ray Powder Diffractometry (XRPD)

XRPD patterns were collected using the PANalytical Empyrean powder X-ray diffractometer (PANalytical Inc., Lelyweg, Netherlands). The powder sample was packed in a zero-background silicon holder and run in reflection mode (Bragg Brentano configuration). The instrument was equipped a Cu Kc source with tube voltage and current of 45 kV and 40 mA respectively. Data was collected at ambient temperature from 3.0 to 40.0 °2θ using a step size of 0.0263°, with a revolution speed of 8 sec. The incident beam path was equipped with a 0.020 soller slit, a fixed 10 anti scatter slit, a fixed incident beam mask of 10 mm, and a programmable divergence slit in automatic mode. A beam knife for linear detectors was used. The diffracted beam was equipped with a 0.020 soller slit, a programmable anti scatter slit in automatic mode, and a nickel K-13 filter. A PIXcel 1D detector was used in the scanning line detector (1D) mode. Data was analyzed using commercial software (JADE®, version 9, Materials Data Inc., Livermore, Calif.).

| Parameters | Reflection mode |
|---|---|
| Model | Empyrean |
| Wavelength | Cu, kα; |
| | Kα₁ (Å): 1.540598, |
| | Kα₂ (Å): 1.544426 |
| | Kα₂/Kα₁ intensity ratio: 0.50 |
| X-Ray tube setting | 45 kV, 40 mA |
| Scan Mode | Continuous |
| Scan range (2 theta degree) | 3 to 40 |
| Divergence slit | ⅛° |
| Scan step time (s) | 46.665 |
| Step size (2 theta degree) | 0.0263 |
| Test time | 5 min 5 sec |

XRPD Method for Dicyclohexylamine Salt of compound 2ac: XRPD patterns were collected using a Rigaku MiniFlex 600 powder X-ray diffractometer (Rigaku Americas Corp., The Woodlands, TX). The powder sample was packed on a zero-background silicon holder and run in Bragg-Brentano reflection geometry. The instrument was equipped a Cu Kα source with tube voltage and current of 40 kV and 15 mA, respectively. Data were collected at ambient temperature from 2.0 to 40.0 °2θ using a step size of 0.02°, scan speed of 4°/min, and sample rotation during acquisition. The incident beam path was equipped with a 2.5° soller slit, a 10 mm incident beam mask, and a divergence slit 0.625°. A variable beam knife for linear detectors was used above the sample. The diffracted beam path utilized an 8 mm scattering slit, 13 mm receiving slit, 2.5° soller slit, Ni KB filter, and a D/teX Ultra detector in scanning line detector (1D) mode. Data were analyzed using HighScore 5.1 (Malvern Panalytical Inc., Westborough, MA).

| | |
|---|---|
| Parameters | Reflection mode |
| Model | Rigaku MiniFlex 600 |
| Wavelength | Cu Kα |
| | 1.5406 Å |
| X-Ray tube setting | 40 kV, 15 mA |
| Scan Mode | Continuous |
| Scan range (2 theta degree) | 2.0 to 40.0 °2θ |
| Divergence slit | 1.25° |
| Scan step time (s) | 4°/min |
| Step size (2 theta degree) | 0.02° |
| Test time | 9.5 min |

### Differential Scanning Calorimetry (DSC)

Approximately 3-8 mg of powder sample was analyzed using a DSC Q2000™ (TA instruments, New Castle, Del.) equipped with a refrigerated cooling accessory. Samples were packed in non-hermetically pans (Tzero™, aluminum pans) and typically heated from 0-200° C. at 10° C./min under dry nitrogen purge. The instrument was calibrated using sapphire (baseline) and indium (temperature and cell constant). The data was analyzed using commercial software (Universal Analysis 2000, version 4.7 A, TA Instruments).

DSC Method for Dicyclohexylamine Salt of compound 2ac: Approximately 2-5 mg of powder sample was analyzed using a DSC Q2000 (TA Instrument) equipped with a chiller. Samples were packed in non-hermetically pans (Tzero™, aluminum pans) and typically heated from 0-350° C. at 10° C./min under dry nitrogen purge. The instrument was calibrated using a aluminum pan (baseline) and indium (temperature and cell constant). The data were analyzed using commercial software, TA Universal Analysis.

### Thermogravimetry (TGA)

In a thermogravimetric analyzer (Discovery TGA, TA instruments), 3-5 mg of compound samples were heated in an open aluminum pan from room temperature to 350° C. at a heating rate of 10° C./min under dry nitrogen purge. Temperature calibration was performed using Alumel® and Nickel. Standard weights of 100 mg and 1 μm were used for weight calibration.

TGA Method for Dicyclohexylamine Salt of compound 2ac: In a thermogravimetric analyzer (Discovery TGA, TA instruments), 3-5 mg of compound samples were heated in an open aluminum pan from room temperature to 350° C. at a heating rate of 10° C./min under dry nitrogen purge. The data was analyzed using commercial software, TA Universal Analysis.

### Screen of Salts

Starting with free form 2ac, fifty-four salt screening experiments were performed using eighteen bases and three solvents. Table 6 details the results.

TABLE 6
Results of Salt Screen of 2ac

| | | Solvent | | |
|---|---|---|---|---|
| | Base | A | B | C |
| | Ratio base:Free form | IPA | Acetone | EtOAc |
| 0 | Blank | Oil | Oil | Oil |
| 1 | NaOH (2:1) | Amorphous | 3 Peaks | Amorphous |
| 2 | KOH (2:1) | Amorphous | Oil | Oil |
| 3 | Ca(OH)₂ (1:1) | Ca(OH)₂ | Ca(OH)₂ | Ca(OH)₂ |
| 4 | Mg(OH)₂ (1:1) | Mg(OH)₂ | Mg(OH)₂ | Mg(OH)₂ |
| 5 | Choline (2:1) | Oil | Oil | Oil |
| 6 | Ammonia (1:1) | Oil | Oil | Oil |
| 7 | Urea (2:1) | Urea | Urea + peaks | Oil |
| 8 | Meglumine (1:1) | Amorphous | Oil | Meglumine |
| 9 | L-Arginine (1:1) | Oil | Oil | Oil |
| 10 | L-Lysine (1:1) | Lysine salt | Oil | Amorphous |
| | | Type A | | |
| 11 | Tromethamine (1:1) | Oil | Oil | Tromethamine |
| 12 | Theophylline (1:1) | Theophylline | Theophylline | Theophylline |
| 13 | Deanol (1:1) | Oil | Oil | Oil |
| 14 | Diethylamine (1:1) | Oil | Oil | Oil |
| 15 | Diethanolamine (1:1) | Oil | Oil | Oil |
| 16 | Triethanolamine (1:1) | Oil | Oil | Oil |
| 17 | Piperazine (1:1) | Oil | Piperazine salt | Piperazine salt |
| | | | type A | type A |
| 18 | Diisopropylamine (1:1) | Oil | Oil | Oil |

Scale up of lysine salt: Weighed 100.0 mg free form 2ac into a 5-mL glass vial. Added 1 mL IPA to dissolve the sample. Weighed 29.2 mg L-lysine into a 3-mL glass vial and added 1.5 mL IPA to obtain lysine suspension. Stirred at room temperature (~800 rpm) and added the free form 2ac solution into the lysine suspension in 15 minutes. Then stirred at room temperature for 1 day and solids were obtained. Then added 2 mL IPA into the suspension and stirred at room temperature for 1 day. Isolated solids by centrifugation (10000 rpm, 2 min) and dried the solids under vacuum at room temperature for ~3 hrs and at 50° C. for ~2 hrs. Collected the solids and a total of 80 mg 2ac lysine salt was obtained (Yield: 61.9%). XRPD data for the 2ac lysine salt shown in FIG. 1 and in Table 7. TGA and DSC data for the 2ac lysine salt shown in FIG. 2. TGA showed a weight loss of 4.09% from between 18.9° C. to 150° C. DSC showed two endotherms at 134.6° C. and 194.4° C.

TABLE 7
Representative XRPD Peaks
for 2ac Lysine Salt

| 2-Theta | d |
|---|---|
| (°2θ) | (Å) |
| 3.8 | 23.02 |
| 6.9 | 12.74 |
| 7.6 | 11.60 |
| 8.1 | 10.90 |
| 8.9 | 9.93 |
| 10.3 | 8.57 |
| 11.3 | 7.79 |
| 13.3 | 6.66 |
| 13.9 | 6.39 |
| 15.2 | 5.85 |
| 16.6 | 5.34 |
| 17.3 | 5.13 |
| 18.2 | 4.88 |
| 20.9 | 4.24 |
| 22.0 | 4.04 |
| 22.4 | 3.97 |
| 24.1 | 3.70 |
| 25.2 | 3.54 |
| 26.3 | 3.39 |
| 27.4 | 3.25 |
| 31.7 | 2.82 |
| 32.8 | 2.73 |
| 34.7 | 2.58 |

Scale up of piperazine salt: Weighed 100.0 mg free form 2ac into a 5-mL glass vial. Added 1 mL EtOAc to dissolve the sample. Weighed 17.4 mg piperazine into a 3-mL glass vial and added 1.5 mL EtOAc to dissolve the base. Added the piperazine solution into the free form 2ac solution over 1.5 hrs. After stirring for 1.5 hrs, no solid was observed, so 1.0 mg of the 2ac piperazine salt was added into the solution as seeds. The mixture was then stirred at room temperature for ~2 hrs and solids were observed. The solids were isolated by centrifugation (10000 rpm, 2 min) and then dried at 50° C. under vacuum for ~2 hrs. Collected the solids for and a total of 56 mg 2ac piperazine salt was obtained (Yield: 47.7%). XRPD data for the 2ac piperazine salt shown in FIG. 3 and Table 8. TGA and DSC for the 2ac piperazine salt shown in FIG. 4. TGA showed a weight loss of 4.93% from between 27.6° C. to 100° C. DSC showed an endotherm at 89.8° C.

TABLE 8
Representative XRPD Peaks
for 2ac Piperazine Salt

| 2-Theta | d |
|---|---|
| (°2θ) | (Å) |
| 11.0 | 8.06 |
| 12.6 | 7.01 |
| 13.4 | 6.62 |
| 14.2 | 6.25 |
| 16.8 | 5.28 |
| 17.5 | 5.08 |
| 18.1 | 4.89 |
| 19.0 | 4.67 |
| 19.7 | 4.50 |
| 20.4 | 4.35 |
| 21.2 | 4.20 |
| 22.0 | 4.03 |
| 22.6 | 3.94 |
| 23.6 | 3.77 |
| 24.6 | 3.62 |
| 25.3 | 3.52 |
| 26.5 | 3.36 |
| 26.9 | 3.31 |
| 27.4 | 3.26 |
| 28.2 | 3.17 |
| 30.6 | 2.92 |
| 31.9 | 2.81 |
| 36.8 | 2.44 |

Scale up of dicyclohexylamine salt: To a solution of 100 g of free form 2ac (solution in MTBE approximately 29.5%-w/w) in a 500 mL reactor was added 10 mL MeOH. The solution was then diluted with 100 mL MTBE/MeOH 9:1 v/v. The reaction mixture was heated to 40° C. to 45° C. and dicyclohexylamine (1.1 equiv) was added over 1 hour. After 20 minutes, the mixture was seeded with 2ac dicyclohexylamine salt and the suspension was heated for about 1 hour at 40° C. to 45° C. The suspension was cooled over about 1 hour to 20° C. to 30° C. (target: 25° C.). The suspension was then filtered and the cake washed with 50 mL MTBE/MeOH 9:1 v/v. The solid was dried at 40° C. under vacuo to obtain 35.6 g of 2ac dicyclohexylamine salt as a white solid 88.6% yield, purity: 99.7 A %, chiral purity: >99:1 er.

Alternatively, the dicyclohexylamine salt was prepared as follows: The crude free form 2ac solution (1366 g, approximately 30%-w/w solution in MTBE, 805 mmol, 1.0 eq) was charged in a six-liter reactor. MTBE (960 mL) and methanol (240 mL) were added, and the solution was heated up to 45° C. Dicyclohexylamine (155 g, 846 mmol, 1.05 eq) was diluted in MTBE (240 mL). A 20% aliquot of the dicyclohexylamine solution was added at 45° C. and the turbid solution was seeded with 2ac dicyclohexylamine salt (0.5 g). The light suspension was aged for 0.5 h. The remaining 80% of the dicyclohexylamine solution was dosed over 4 h at 45° C. The suspension was cooled down to 20° C. over 1 h and aged for 1 h. The solid was filtered off and the filter cake was washed with a mixture of MTBE (1200 mL) and methanol (120 mL). The loss of product in the crystallization liquor was 3.1% yield of theory. The wet 2ac dicyclohexylamine salt was dried at 40° C. in the vacuum tray dryer. 486 g 2ac dicyclohexylamine salt was obtained in 88% yield, purity: 99.3 A %, chiral purity: >99:1 er.

Dicyclohexylamine salts of compounds 2ag and 2ah were prepared by the methods detailed above for the 2ac dicyclohexylamine salt. Compound characterization data for the dicyclohexylamine salts of compounds 2ag and 2ah are shown below.

XRPD data for the 2ac dicyclohexylamine salt shown in FIG. 5 and Table 9. TGA and DSC for the 2ac dicyclohexylamine salt shown in FIG. 6. TGA showed a weight loss of 3.58% from between 25° C. to 130° C. DSC showed an endotherm at 177.01° C.

TABLE 9
Representative XRPD Peaks for
2ac Dicyclohexylamine Salt

| 2-Theta | d |
|---|---|
| (°2θ) | (Å) |
| 5.2 | 17.07 |
| 7.5 | 11.73 |
| 8.1 | 10.88 |
| 8.7 | 10.12 |
| 10.3 | 8.59 |
| 12.6 | 7.03 |
| 13.7 | 6.46 |
| 15.0 | 5.90 |
| 15.4 | 5.75 |
| 16.2 | 5.46 |
| 16.5 | 5.36 |
| 17.6 | 5.02 |
| 17.7 | 5.00 |
| 18.8 | 4.71 |
| 19.2 | 4.61 |
| 19.9 | 4.45 |
| 20.5 | 4.32 |
| 21.0 | 4.23 |
| 21.3 | 4.17 |
| 22.4 | 3.97 |
| 23.1 | 3.84 |
| 23.9 | 3.72 |
| 25.0 | 3.56 |
| 25.7 | 3.47 |
| 26.3 | 3.39 |
| 27.5 | 3.24 |
| 27.8 | 3.20 |
| 28.1 | 3.17 |
| 28.7 | 3.11 |
| 29.1 | 3.06 |
| 30.1 | 2.97 |
| 30.5 | 2.93 |
| 31.1 | 2.87 |
| 32.1 | 2.79 |
| 32.6 | 2.74 |
| 33.0 | 2.71 |
| 34.0 | 2.63 |
| 34.4 | 2.61 |
| 35.3 | 2.54 |
| 35.8 | 2.50 |
| 36.4 | 2.47 |
| 36.9 | 2.43 |
| 38.1 | 2.36 |
| 39.0 | 2.30 |

### General Conditions and Data

### Enantiomeric Excess was Determined by HPLC Using One of the Methods Below:

HPLC conditions A

| Column | Chiracel OZ-3R, 4.6 × 150 mm, | |
|---|---|---|
| | 3 μm | |
| Column temperature | 40° C. | |
| Flow rate | 1 mL/min | |
| Injection volume | 3 μL | |
| Detection wavelength | 210 nm | |
| Mobile Phase | A : 0.1% Formic acid in water | |
| | B: 0.1% Formic acid in ACN | |
| | Time | % B |
| Gradient | (min) | Mobile Phase |
| | 0 | 30 |
| | 2 | 30 |
| | 7 | 85 |
| | 10 | 85 |
| | 10.10 | 30 |
| | 14.00 | 30 |

HPLC conditions B

| Column | Chiracel OZ-3R, 4.6 × 150 mm, 3 μm | |
|---|---|---|
| Column temperature | 40° C. | |
| Flow rate | 1 mL/min | |
| Injection volume | 3 μL | |
| Detection wavelength | 210 nm | |
| Mobile Phase | A : 0.1% Formic Acid in water | |
| | B: 0.1% Formic Acid in Acetonitrile | |
| | Time | % B |
| Gradient | (min) | Mobile Phase |
| | 0 | 40 |
| | 5 | 40 |
| | 10 | 55 |
| | 11 | 55 |
| | 11.1 | 40 |
| | 14.00 | 40 |

HPLC conditions C

| Column | Chiracel OZ-3R, 4.6 × 150 mm, 3 μm | |
|---|---|---|
| Column temperature | 40° C. | |
| Flow rate | 0.5 mL/min | |
| Injection volume | 1 μL | |
| Detection wavelength | 218 nm | |
| Mobile Phase | A: 0.1% Formic Acid in water | |
| | B: 0.1% Formic Acid in Acetonitrile | |
| | Time | % B |
| Gradient | (min) | Mobile Phase |
| | 0 | 48 |
| | 10 | 48 |
| | 20 | 70 |
| | 22 | 48 |

HPLC conditions D

| Column | Chiracel OZ-3R, 4.6 × 150 mm, 3 μm | |
|---|---|---|
| Column temperature | 40° C. | |
| Flow rate | 1 mL/min | |
| Injection volume | 1 μL | |
| Detection wavelength | 218 nm | |
| Mobile Phase | A 0.1% Formic Acid in water | |
| | B: 0.1% Formic Acid in Acetonitrile | |
| | Time | % B |
| Gradient | (min) | Mobile Phase |
| | 0 | 65 |

HPLC conditions E

| Column | Chiracel OZ-3R, 4.6 × 150 mm, 3 μm | |
|---|---|---|
| Column temperature | 40° C. | |
| Flow rate | 1 mL/min | |
| Injection volume | 1 μL | |
| Detection wavelength | 260 nm | |
| Mobile Phase | A: 0.1% Formic Acid in water | |
| | B: 0.1% Formic Acid in Acetonitrile | |
| | Time | % B |
| Gradient | (min) | Mobile Phase |
| | 0 | 20 |
| | 4 | 20 |
| | 20 | 30 |

HPLC conditions F

| Column | Chiracel OZ-3R, 4.6 × 150 mm, 3 μm | |
|---|---|---|
| Column temperature | 40° C. | |
| Flow rate | 1 mL/min | |
| Injection volume | 1 μL | |
| Detection wavelength | 260 nm | |
| Mobile Phase | A: 0.1% Formic Acid in water | |
| | B: 0.1% Formic Acid in Acetonitrile | |
| | Time | % B |
| Gradient | (min) | Mobile Phase |
| | 0 | 65 |

HPLC conditions G

| Column | Chiracel OZ-3R, 4.6 × 150 mm, 3 μm | |
|---|---|---|
| Column temperature | 40° C. | |
| Flow rate | 1 mL/min | |
| Injection volume | 1 μL | |
| Detection wavelength | 260 nm | |
| Mobile Phase | A: 0.1% Formic Acid in water | |
| | B: 0.1% Formic Acid in Acetonitrile | |
| | Time | % B |
| Gradient | (min) | Mobile Phase |
| | 0 | 40 |
| | 5.0 | 70 |

HPLC conditions H

| Column | Chiracel OZ-3R, 4.6 × 150 mm, 3 μm | |
|---|---|---|
| Column temperature | 40° C. | |
| Flow rate | 1 mL/min | |
| Injection volume | 1 μL | |
| Detection wavelength | 260 nm | |
| Mobile Phase | A: 0.1% Formic Acid in water | |
| | B: 0.1% Formic Acid in Acetonitrile | |
| | Time | % B |
| Gradient | (min) | Mobile Phase |
| | 0 | 50 |
| | 6.5 | 50 |
| | 8 | 60 |

HPLC conditions I

| Column | Chiracel OZ-3R, 4.6 × 150 mm, 3 μm | |
|---|---|---|
| Column temperature | 40° C. | |
| Flow rate | 1 mL/min | |
| Injection volume | 1 μL | |
| Detection wavelength | 260 nm | |
| Mobile Phase | A: 0.1% Formic Acid in water | |
| | B: 0.1% Formic Acid in Acetonitrile | |
| | Time | % B |
| Gradient | (min) | Mobile Phase |
| | 0 | 48 |
| | 10 | 48 |
| | 20 | 70 |
| | 22 | 90 |
| | 24 | 48 |

HPLC conditions J

| Column | Diacel Chiralpak Chiracel OZ-RH, | |
|---|---|---|
| | 4.6 × 150 mm, 5 μm | |
| Column temperature | 35° C. | |
| Flow rate | 1 mL/min | |
| Injection volume | 1 μL | |
| Detection wavelength | 210 nm | |
| Mobile Phase | A: 0.1% Phosphoric acid in water | |
| | B: Acetonitrile | |
| | Time | % B |
| Gradient | (min) | Mobile Phase |
| | 0 | 55 |
| | 20 | 55 |

### Compound Characterization

¹H and ¹³C NMR spectra were recorded in deuterated methanol (MeOD), unless otherwise noted, on a 400 or 500 MHz instrument. Chemical shifts of ¹H NMR spectra are reported in parts per million (ppm) on the δ scale. Data are reported as follows: chemical shift, integration, multiplicity (s=singlet, d=doublet, t=triplet, q=quartet, and m=multiplet) and coupling constant in hertz (Hz). Chemical shifts of ¹³C NMR spectra are reported in ppm on the δ scale.

HRMS data were collected on a Thermo Orbitrap FUSION equipped with an HESI source in positive ionization mode. The samples were introduced into the mass spectrometer using an Ultimate 3000 HPLC with an Agilent Extended-C18 (2.1-50 mm, 1.8 μm) column and a gradient from 5% to 95% acetonitrile in water with 0.1% formic acid in both channels at a flow rate of 0.6 mL/min.

| Compound Name | Structure | Data |
|---|---|---|
| (S)-2-(((benzyloxy)carbonyl) (methyl)amino)-2-(4-hydroxy phenyl)acetic acid (2i) | 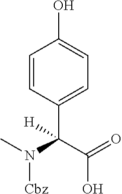 [SMILES: CN(C(=O)OCc1ccccc1)[C@H](C(=O)O)c1ccc(O)cc1] | ¹H NMR (500 MHz, MeOD) δ 7.42-7.25 (m, 5H), 7.16-7.01 (m, 2H), 6.79 (d, J = 7.0 Hz, 2H), 5.8¹ (s(br), 1H), 5.17 (s, 2H), 2.69 (s, 3H) ¹³C NMR (126 MHz, MeOD) δ 172.8, 157.5, 157¹, 136.6, 130.3, 128.1, 127.7, 127.4, 115.1, 67.3, |
| | | 62.2, 29.8¹ |
| | | HRMS HESI (m/z): |
| | | [M + H]⁺ calcd for |
| | | C₁₇H₁₈NO₅, 316.1185; |
| | | found 316.1168. |
| | | The enantiomeric |
| | | excess was determined |
| | | by HPLC conditions |
| | | A, Tr (major) 4.81 |
| | | min, Tr(minor) 5.11 |
| | | min. |
| (S)-2-(((benzyloxy)carbonyl) (methyl)amino)-2-(3-fluoro-4- hydroxyphenyl)acetic acid (2j) | 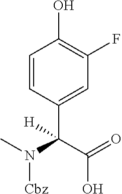 [SMILES: CN(C(=O)OCc1ccccc1)[C@H](C(=O)O)c1ccc(O)c(F)c1] | ¹H NMR (500 MHz, MeOD) δ 7.40-7.25 (m, 5H), 7.00 (m(br), 1H), 6.92 (m(br), 2H), 5.80 (s(br), 1H), 5.18 (s, 2H), 2.73 (s, 3H) ¹³C NMR (126 MHz, MeOD) δ 172.0, 156.7¹, 152.2, 150.3, 145.1 (d, J = 5 Hz), 136.5, 128.2, 127.7, 127.4, 125.8¹, 125.2 |
| | | (d, J = 3.78 Hz), 117.4 |
| | | (d, J = 3.78 Hz), 116.5 |
| | | (d, J = 25 Hz), 67.3, |
| | | 61.9, 30.4¹ |
| | | HRMS HESI (m/z): |
| | | [M + H]⁺ calcd for |
| | | C₁₇H₁₇FNO₅, |
| | | 334.1091; found |
| | | 334.1074. |
| | | The enantiomeric |
| | | excess was determined |
| | | by HPLC conditions |
| | | A, Tr (major) 4.87 |
| | | min, Tr(minor) 5.23 |
| | | min. |
| (S)-2-(((benzyloxy)carbonyl) (methyl)amino)-2-(3-chloro-4- hydroxyphenyl)acetic acid (2k) | 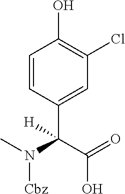 [SMILES: CN(C(=O)OCc1ccccc1)[C@H](C(=O)O)c1ccc(O)c(Cl)c1] | ¹H NMR (500 MHz, MeOD) δ 7.40-7.26 (m, 5H), 7.23 (s (br), 1H), 7.06 (d, J = 8.5 Hz, 1H), 6.92 (d, J = 8.4 Hz, 1H), 5.80 (s (br), 1H), 5.18 (s, 2H), 2.73 (s, 3H) ¹³C NMR (126 MHz, MeOD) δ 172.0, 156.8¹, 153.2, 136.5, 130.4, 128.6, 128.2, |
| | | 127.7, 127.4, 126.4, |
| | | 120.4, 116.3, 67.4, |
| | | 61.8, 30.1¹ |
| | | HRMS HESI (m/z): |
| | | [M + H]⁺ calcd for |
| | | C₁₇H₁₇ClNO₅, |
| | | 350.0795; found |
| | | 350.0778. |
| | | The enantiomeric |
| | | excess was determined |
| | | by HPLC conditions |
| | | A, Tr (major) 5.38 |
| | | min, Tr(minor) 5.73 |
| | | min. |
| (S)-2-(((benzyloxy)carbonyl) (methyl)amino)-2-(3-bromo- 4-hydroxyphenyl)acetic acid (2a) | 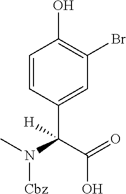 [SMILES: CN(C(=O)OCc1ccccc1)[C@H](C(=O)O)c1ccc(O)c(Br)c1] | ¹H NMR (500 MHz, MeOD) δ: 7.47-7.23 (m, 6H), 7.15-7.10 (m, 1H), 6.87 (d, J = 8.4 Hz, 1H), 5.75 (s (br), 1H), 5.17 (s (br), 2H), 2.75 (s, 3H) ¹³C NMR (126 MHz, MeOD) δ 175.5, 159.0¹, 153.7, 136.9, 133.8, 130.0, 129.6, 128.1, 127.5, 127.3, |
| | | 115.6, 109.2, 67.0, |
| | | 64.1, 30.51 |
| | | HRMS HESI (m/z): |
| | | [M + H]⁺ calcd for |
| | | C₁₇H₁₇BrNO₅, |
| | | 394.0290; found |
| | | 394.0270. |
| | | The enantiomeric |
| | | excess was determined |
| | | HPLC conditions B, Tr |
| | | (major) 3.70 min, |
| | | Tr(minor) 4.40 min. |
| (S)-2-(((benzyloxy)carbonyl) (butyl)amino)-2-(3-bromo-4- hydroxyphenyl)acetic acid (2l) | 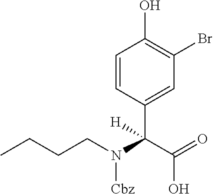 [SMILES: CCCCN(C(=O)OCc1ccccc1)[C@H](C(=O)O)c1ccc(O)c(Br)c1] | ¹H NMR (500 MHz, DMSO_{d6}) δ: 7.45-7.21 (m, 6H), 7.15-7.10 (m, 1H), 6.93 (d, J = 8.4 Hz, 1H), 5.43 (s (br), 1H), 5.11 (s, 2H), 3.29-3.18 (m, 1H), 3.07-2.95 (m, 1H), 1.41-1.30 (m, 1H), 1.12-0.97 (m, 3H), 0.76-0.62 (m, 3H); ¹³C NMR (126 MHz, |
| | | DMSO_{d6}) δ 171.8¹, |
| | | 156.1¹, 154.4, 136.9, |
| | | 133.9, 130.9, 128.7, |
| | | 128.2, 127.7, 116.4, |
| | | 109.4, 66.9, 62.5¹, |
| | | 46.1¹, 30.9¹, 19.7, |
| | | 13.8; |
| | | HRMS ESI (m/z): |
| | | [M + H]⁺ calcd for |
| | | C₂₀H₂₃BrNO₅, |
| | | 436.0754; found |
| | | 436.0751. |
| | | The enantiomeric |
| | | excess was determined |
| | | HPLC conditions C, Tr |
| | | (major) 7.32 min, |
| | | Tr(minor) 8.49 min. |
| (S)-2-(((benzyloxy)carbonyl) (methyl)amino)-2-(4-hydroxy- 3-methylphenyl)acetic acid (2m) | 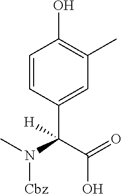 | ¹H NMR (500 MHz, MeOD) δ 7.40-7.26 (m, 5H), 6.97 (s, 1H), 6.91 (d, J = 8.2 Hz, 1H), 6.74 (d, J = 8.2 Hz, 1H), 5.77¹ (s(br), 5.17 (s, 2H), 2.69 (s, 3H), 2.17 (s, 3H) ¹³C NMR (126 MHz, MeOD) δ 172.9, 157.4, 155.6, 136.6, 131.4, 128.1, 127.7, |
| | | 127.5, 127.4, 124.8, |
| | | 114.2, 67.2, 62.2, 29.8, |
| | | 14.8 |
| | | HRMS HESI (m/z): |
| | | [M + H]⁺ calcd for |
| | | C₁₈H₂₀NO₅, 330.1341; |
| | | found 330.1325. |
| | | The enantiomeric |
| | | excess was determined |
| | | by HPLC conditions |
| | | A, Tr (major) 5.38 |
| | | min, Tr(minor) 5.71 |
| | | min. |
| (S)-2-(((benzyloxy)carbonyl) (methyl)amino)-2-(3-bromo- 4,5-dihydroxyphenyl)acetic acid (2n) | 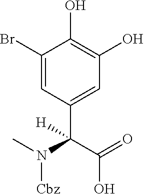 [SMILES: CN(C(=O)OCc1ccccc1)[C@H](C(=O)O)c1cc(O)c(O)c(Br)c1] | ¹H NMR (500 MHz, MeOD) δ 7.40-7.27 (m, 5H), 6.87 (s (br), 1H), 6.71 (s (br), 1H), 5.73¹ (s(br), 1H), 5.18 (s, 2H), 2.73 (s, 3H) ¹³C NMR (126 MHz, MeOD) δ 172.1, 156.7¹, 146.1, 143.3, 136.5, 128.2, 127.7, 127.4, 126.4, 123.7, 114.8, 109.2, 67.3, |
| | | 61.8, 30.0¹ |
| | | HRMS HESI (m/z): |
| | | [M + H]⁺ calcd for |
| | | C₁₇H₁₇BrNO₆, |
| | | 410.0239; found |
| | | 410.0219. |
| | | The enantiomeric |
| | | excess was determined |
| | | by HPLC conditions |
| | | A, Tr (major) 4.71 |
| | | min, Tr(minor) 5.03 |
| | | min. |
| (S)-2-(((benzyloxy)carbonyl) (methyl)amino)-2-(3,5- dibromo-4-hydroxyphenyl) acetic acid (2o) | 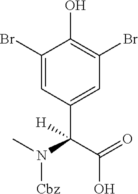 | ¹H NMR (500 MHz, MeOD) δ 7.52-7.17 (m, 7H), 5.77 (s (br), 1H), 5.18 (s, 2H), 2.78 (s, 3H) ¹³C NMR (126 MHz, MeOD) δ 171.2, 156.7¹, 151.1, 136.4, 132.7, 128.6, 128.2, 127.8, 127.5, 110.7, 67.5, 61.4, 30.61 HRMS HESI (m/z): [M + H]⁺ calcd for |
| | | C₁₇H₁₆Br₂NO₅, |
| | | 471.9395; found |
| | | 471.9372. |
| | | The enantiomeric |
| | | excess was determined |
| | | by HPLC conditions |
| | | A, Tr (major) 6.34 |
| | | min, Tr(minor) 6.59 |
| | | min. |
| (S)-2-(((benzyloxy)carbonyl) (methyl)amino)-2-(4-hydroxy- 2-methylphenyl)acetic acid (2p) | 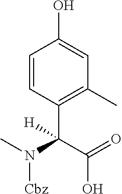 | ¹H NMR (500 MHz, MeOD) δ 7.53-7.17 (m, 5H), 6.96 (d, J = 8.4 Hz, 1H), 6.68 (s, 1H), 6.62 (dd, J = 8.4, 2.5 Hz, 1H), 5.89¹ (s(br), 1H), 5.18 (s(br), 2H), 2.61 (s(br), 3H)¹, 2.13 (s(br), 3H)1 ¹³C NMR (126 MHz, MeOD) δ 173.5, 157.6, 156.9¹, 139.4, |
| | | 136.71, 130.0¹, 128.1, |
| | | 127.7, 127.5, 127.3, |
| | | 123.0¹, 117.2, 112.3, |
| | | 67.3¹, 67.2, 59.8, |
| | | 29.3¹, 17.8 |
| | | HRMS HESI (m/z): |
| | | [M + H]⁺ calcd for |
| | | C₁₈H₂₀NO₅, 330.1341; |
| | | found 330.1327. |
| | | The enantiomeric |
| | | excess was determined |
| | | by HPLC conditions |
| | | A, Tr (major) 5.23 |
| | | min, Tr(minor) 5.61 |
| | | min. |
| (S)-2-(((benzyloxy)carbonyl) (methyl)amino)-2-(2-fluoro-4- hydroxyphenyl)acetic acid (2q) | 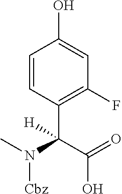 [SMILES: CN(C(=O)OCc1ccccc1)[C@H](C(=O)O)c1ccc(O)cc1F] | ¹H NMR (500 MHz, MeOD) δ 7.45-7.24 (m, 5H), 7.16-7.05 (m, 1H), 6.65 (s(br), 1H), 6.59 (dd, J = 12.0, 2.4 Hz), 5.99¹ (s(br), 1H), 5.19 (s(br), 2H), 2.77 (s(br), 3H) ¹³C NMR (126 MHz, MeOD) δ 172.0, 162.9, 160.9, 159.8¹ 156.7¹, 136.5, 131.3¹, |
| | | 128.1, 127.7, 127.3, |
| | | 112.2¹, 111.2¹, 102.5¹, |
| | | 67.3, 56.8¹, 29.9¹ |
| | | HRMS HESI (m/z): |
| | | [M + H]⁺ calcd for |
| | | C₁₇H₁₇FNO₅, |
| | | 334.1090; found |
| | | 334.1075. |
| | | The enantiomeric |
| | | excess was determined |
| | | by HPLC conditions |
| | | A, Tr (major) 4.86 |
| | | min, Tr(minor) 5.27 |
| | | min. |
| (S)-2-(((benzyloxy)carbonyl) (methyl)amino)-2-phenyl acetic acid (2r) | 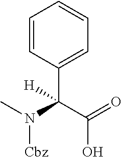 [SMILES: CN(C(=O)OCc1ccccc1)[C@H](C(=O)O)c1ccccc1] | ¹H NMR (500 MHz, MeOD) δ 7.56-7.12 (m, 10H), 5.92¹ (s(br), 1H), 5.19 (s, 2H), 2.71 (s, 3H) ¹³C NMR (126 MHz, MeOD) δ 172.2, 157.4¹, 136.5, 134.5¹, 128.9, 128.4, 128.2, 127.7, 127.4, 67.4, |
| | | 62.6, 30.1 |
| | | HRMS HESI (m/z): |
| | | [M + H]⁺ calcd for |
| | | C₁₇H₁₈NO₄, 300.1235; |
| | | found 300.1222. |
| | | The enantiomeric |
| | | excess was determined |
| | | by HPLC conditions |
| | | A, Tr (major) 6.38 |
| | | min, Tr(minor) 6.78 |
| | | min. |
| (S)-2-(((benzyloxy)carbonyl) (methyl)amino)-2-(3-bromo phenyl)acetic acid (2s) | 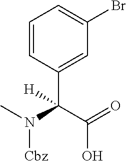 [SMILES: CN(C(=O)OCc1ccccc1)[C@H](C(=O)O)c1cccc(Br)c1] | ¹H NMR (500 MHz, MeOD) δ 7.64-7.14 (m, 9H), 5.87 (s(br), 1H), 5.19 (s, 2H), 2.76 (s, 3H) ¹³C NMR (126 MHz, MeOD) δ 171.3, 156.9¹, 136.8¹, 131.7, 131.2, 130.1, 128.2, 127.8, 127.6, 127.5, |
| | | 122.1, 67.5, 62.2, |
| | | 30.5¹ |
| | | HRMS HESI (m/z): |
| | | [M + H]⁺ calcd for |
| | | C₁₇H₁₇BrNO₄, |
| | | 378.0341; found |
| | | 378.0320. |
| | | The enantiomeric |
| | | excess was determined |
| | | by HPLC conditions |
| | | A, Tr (major) 6.88 |
| | | min, Tr(minor) 7.27 |
| | | min. |
| (S)-2-(4-(benzyloxy)-3- bromophenyl)-2-(((benzyloxy) carbonyl)(methyl)amino) acetic acid (2t) | 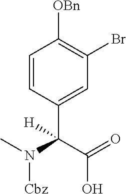 [SMILES: CN(C(=O)OCc1ccccc1)[C@H](C(=O)O)c1ccc(OCc2ccccc2)c(Br)c1] | ¹H NMR (500 MHz, MeOD) δ 7.49 (s(br), 1H), 7.47 (d, J = 6.5 Hz, 2H), 7.40-7.26 (m, 8H), 7.26-7.16 (m, 1H), 7.09 (d, J = 8.5 Hz, 1H), 5.811 (s(br), 1H), 5.17 (s, 2H), 2.74 (s, 3H) ¹³C NMR (126 MHz, MeOD) δ 171.7, 157.3¹, 155.1, 136.47, |
| | | 136.54, 133.6, 129.3, |
| | | 128.4, 128.1¹, 127.8, |
| | | 127.6, 127.4, 126.9, |
| | | 113.5, 111.8, 70.5, |
| | | 67.4, 61.7, 29.4 |
| | | HRMS HESI (m/z): |
| | | [M + H]⁺ calcd for |
| | | C₂₄H₂₃BrNO₅, |
| | | 484.0759; found |
| | | 484.0741. |
| | | The enantiomeric |
| | | excess was determined |
| | | by HPLC conditions |
| | | A, Tr (major) 7.95 |
| | | min, Tr(minor) 8.32 |
| | | min. |
| (S)-2-(4-(benzyloxy)-3- hydroxy-5-iodophenyl)-2- (((benzyloxy)carbonyl) (methyl)amino)acetic acid (2u) | 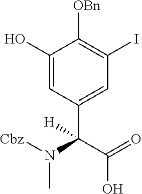 [SMILES: CN(C(=O)OCc1ccccc1)[C@H](C(=O)O)c1cc(O)c(OCc2ccccc2)c(I)c1] | ¹H NMR (500 MHz, CDCl₃) δ: 7.51-7.47 (m (br), 2H), 7.44-7.27 (m, 9H), 6.92-6.82 (m (br), 1H), 5.98 (s (br), 1H), 5.20 (s, 2H), 5.03 (s, 2H), 2.81 (s, 3H) HRMS ESI (m/z): [M + NH₄]⁺ calcd for C₂₄H₂₆IN₂O₆, 565.0835; found 565.0833. The |
| | | enantiomeric excess |
| | | was determined HPLC |
| | | conditions D, Tr |
| | | (major) 2.61 min, |
| | | Tr(minor) 2.88 min. |
| N-((benzyloxy)carbonyl)-N- methyl-L-phenylalanine (2v)² | 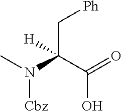 [SMILES: CN(C(=O)OCc1ccccc1)[C@@H](Cc1ccccc1)C(=O)O] | ¹H NMR (500 MHz, MeOD) δ 7.39-7.09 (m, 10H), 5.12-4.82 (m, 3H), 3.38-3.27 (m, 1H), 3.13-2.99 (m, 1H), 2.79¹ (s,(br), 3H) ¹³C NMR (126 MHz, |
| | | MeOD) δ 172.6¹, |
| | | 156.7¹, 137.5¹, 136.5¹, |
| | | 128.5¹, 128.15¹, |
| | | 128.09, 127.6¹, 127.3¹ |
| | | 126.3¹, 66.9¹, 60.7¹, |
| | | 34.5¹, 31.3¹ |
| | | HRMS HESI (m/z): |
| | | [M + H]⁺ calcd for |
| | | C₁₈H₂₀NO₄, 314.1392; |
| | | found 314.1377. The |
| | | enantiomeric excess |
| | | was determined by to |
| | | be >95% ee by |
| | | comparison of α_{D} with |
| | | literature.2 [α]_{D literature}: |
| | | +112.7 (c 1, CHCl₃); |
| | | [a]_{D Experimental} +126.6 |
| | | (c 0.923, CHCl₃). |
| ((S)-2-(((benzyloxy)carbonyl) (methyl)amino)-2- cyclopropylacetic acid (2w) | 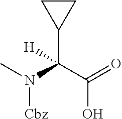 [SMILES: CN(C(=O)OCc1ccccc1)[C@H](C(=O)O)C1CC1] | ¹H NMR (500 MHz, MeOD) δ 7.23-6.94 (m, 5H), 4.891 (s(br), 1H), 3.53 (dd, J = 9.8 Hz, 1H), 2.83 (s, 3H), 1.08-0.96 (m, 1H), 0.61-0.50 (m, 1H), 0.41-0.28 (m, 2H), |
| | | 0.11-0.04 (m, 1H) |
| | | ¹³C NMR (126 MHz, |
| | | MeOD) δ 172.3, |
| | | 155.9¹, 135.7¹, 137.2, |
| | | 126.8, 126.5, 66.2, |
| | | 63.3¹, 29.5¹, 10.0¹, 4.4, |
| | | 1.5¹ |
| | | HRMS HESI (m/z): |
| | | [M + H]⁺ calcd for |
| | | C₁₄H₁₈NO₄, 264.1230; |
| | | found 264.1230. The |
| | | enantiomeric excess |
| | | was determined HPLC |
| | | conditions A, Tr |
| | | (major) 5.91 min, |
| | | Tr(minor) 6.40 min. |
| (S)-2-(((benzyloxy)carbonyl) (methyl)amino)-2- cyclohexylacetic acid (2x) | 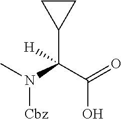 [SMILES: CN(C(=O)OCc1ccccc1)[C@H](C(=O)O)C1CC1] | ¹H NMR (500 MHz, MeOD) δ 7.41-7.27 (m, 5H), 5.19-5.12 (m, 2H), 4.891 (s(br), 1H), 4.451 (d, J = 10.5 Hz, 1H), 2.921 (s (br), 3H), 1.96-1.54 (m, 6H), 1.39-1.15 (m, 3H), |
| | | 1.15-0.86 (m, 2H) |
| | | ¹³C NMR (126 MHz, |
| | | MeOD) δ 172.3¹, |
| | | 157.2¹, 136.6¹, 128.1¹, |
| | | 127.7, 127.4¹, 67.2, |
| | | 63.2¹, 36.5¹, 30.1¹, |
| | | 29.4¹, 25.9¹, 25.4¹ |
| | | HRMS HESI (m/z): |
| | | [M + H]⁺ calcd for |
| | | C₁₇H₂₄NO₄, 306.1705; |
| | | found 306.1691. The |
| | | enantiomeric excess |
| | | was determined HPLC |
| | | conditions A, Tr |
| | | (major) 7.07 min, |
| | | Tr(minor) 7.49 min. |
| (S)-2-(((benzyloxy)carbonyl) (methyl)amino)-2-(pyridin-3- yl)acetic acid (2y) | 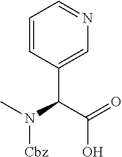 [SMILES: CN(C(=O)OCc1ccccc1)[C@H](C(=O)O)c1cccnc1] | ¹H NMR (500 MHz, D₂O) δ 8.45-8.36 (m, 1H), 7.76-7.68 (m, 2H), 7.48-7.29 (m, 5H), 5.65 (s, 1H), 5.15 (s, 2H), 2.77 (s, 3H) ¹³C NMR (126 MHz, D₂O) δ 175.2, 157.6, 149.2, 147.9, 137.9, 136.1, 133.4, 128.6, |
| | | 128.2, 127.7, 124.1, |
| | | 67.8, 63.61, 31.1¹ |
| | | HRMS ESI (m/z): |
| | | [M + H]⁺ calcd for |
| | | C₁₆H₁₇N₂O₄, |
| | | 301.1183; found |
| | | 301.1182. The |
| | | enantiomeric excess |
| | | was determined HPLC |
| | | conditions E, Tr |
| | | (major) 3.01 min, |
| | | Tr(minor) 3.42 min. |
| (S)-2-((tert- butoxycarbonyl)(methyl) amino)-2-phenylacetic acid (2z) | 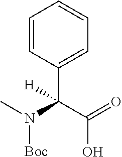 [SMILES: CN(C(=O)OC(C)(C)C)[C@H](C(=O)O)c1ccccc1] | ¹H NMR (MeOD) δ: 7.33-7.17 (m, 5H), 5.701 (s(br), 1H), 2.54 (s, 3H), 1.40 (s, 9H) ¹³C NMR (126 MHz, MeOD) δ 172.2¹, 156.3¹, 134.9, 128.8, 128.3, 128.0, 80.7¹, 70.0¹, 30.4¹, 27.3¹ The enantiomeric |
| | | excess was determined |
| | | by HPLC conditions |
| | | A, Tr (major) 5.95 |
| | | min, Tr(minor) 6.20 |
| | | min. |
| (S)-2-(3-(benzyloxy)-4- hydroxy-5-iodophenyl)-2- (((benzyloxy)carbonyl)(methyl) amino)acetic acid (2aa) | 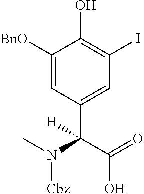 [SMILES: CN(C(=O)OCc1ccccc1)[C@H](C(=O)O)c1cc(I)c(O)c(OCc2ccccc2)c1] | HRMS ESI (m/z): [M + H]⁺ calcd for C₂₄H₂₃INO₆, 548.0565; found 548.0561. The enantiomeric excess was determined HPLC conditions F, Tr (major) 2.59 min, Tr(minor) 3.20 min. |
| (S)-2- (((benzyloxy)carbonyl)(methyl) amino)-2-(3-((R)-3-((tert- butoxycarbonyl)amino)-2- hydroxypropoxy)-4-hydroxy- 5-iodophenyl)acetic acid (2ab) | 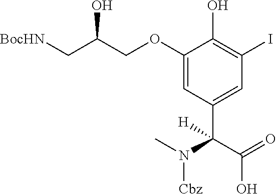 [SMILES: CN(C(=O)OCc1ccccc1)[C@H](C(=O)O)c1cc(I)c(O)c(OC[C@H](O)CNC(=O)OC(C)(C)C)c1] | HRMS ESI (m/z): [M + H]⁺ calcd for C₂₅H₃₁IN₂O₉, 631.1147; found 631.1151. The enantiomeric excess was determined HPLC conditions G, Tr (major) 1.39 min, Tr(minor) 4.30min. |
| (S)-2-(4-(benzyloxy)-3- bromo-5-hydroxyphenyl)-2- (((benzyloxy)carbonyl)(methyl) amino)acetic acid (2ac) | 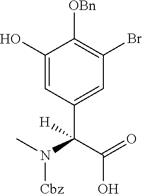 [SMILES: CN(C(=O)OCc1ccccc1)[C@H](C(=O)O)c1cc(O)c(OCc2ccccc2)c(Br)c1] | ¹H NMR (400 MHz, CD₃OD) δ: 13.25 (s, 1H), 10.18 (s, 1H), 7.55-7.50 (m, 2H), 7.42-7.29 (m, 8H), 6.96-6.92 (m, 1H), 6.89-6.85 (m, 1H), 5.66 (s, 1H), 5.14 (s, 2H), 5.00 (s, 2H), 2.70 (s, 3H) HRMS ESI (m/z): [M + H]⁺ calcd for |
| | | C₂₄H₂₃BrNO₆, |
| | | 500.0703; found |
| | | 500.0700. The |
| | | enantiomeric excess |
| | | was determined HPLC |
| | | conditions H, Tr |
| | | (major) 5.04 min, |
| | | Tr(minor) 6.08min. |
| (S)-2- (((benzyloxy)carbonyl)(methyl) amino)-2-(3-bromo-5- hydroxy-4-((2- (trimethylsilyl)ethoxy)methoxy) phenyl)acetic acid (2ad) | 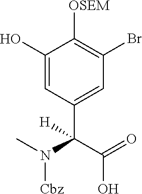 | HRMS ESI (m/z): [M − SEM + H]⁺ calcd for C₁₇H₁₇INO₆, 458.0095; found 458.0089. The enantiomeric excess was determined HPLC conditions I, Tr (major) 17.38 min, Tr(minor) 18.69 min. |
| Dicyclohexylamine (S)-2- (((benzyloxy)carbonyl)(methyl) amino)-2-(3-bromo-5- hydroxy-4-((4- methylbenzyl)oxy)phenyl) acetate (2ag) | 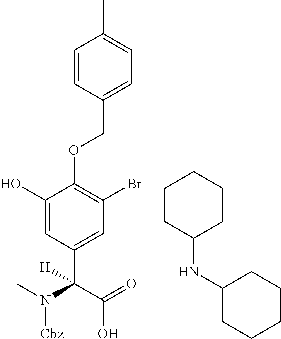 | ¹H NMR (500 MHz, MeOD) δ 7.43-7.25 (m, 7H), 7.17-7.11 (m, 2H), 6.98 (s, 1H), 6.82 (s, 1H), 5.68¹ (s(br), 1H), 5.23-5.11 (m, 2H), 4.94-4.82 (m, 2H), 3.19-3.07 (m, 2H), 2.75 (s, 3H), 2.32 (s, 3H), 2.05-1.97 (m, 4H), 1.89-1.80 (m, 4H), 1.73-1.69 (m, 2H), 1.42-1.12 (m, 10H) ¹³C NMR (126 MHz, MeOD) δ 174.74, 151.38, 142.95, 137.56, 136.80, 135.40, 133.90, 128.57, 128.41, 128.10, 127.56, |
| | | 127.29, 124.14, |
| | | 117.00, 73.94, 66.98, |
| | | 64.12, 53.00, 30.36,¹ |
| | | 29.17, 24.73, 24.05, |
| | | 19.87. The |
| | | enantiomeric excess |
| | | was determined by |
| | | HPLC conditions J, Tr |
| | | (major) 4.62 min, |
| | | Tr(minor) 5.46 min. |
| Dicyclohexylamine (S)-2-(4- ([1,1′-biphenyl]-4- ylmethoxy)-3-bromo-5- hydroxyphenyl)-2- (((benzyloxy)carbonyl) (methyl)amino)acetate (2ah) | 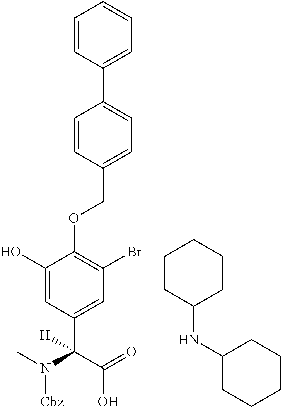 | ¹H NMR (500 MHz, DMSO_{d6}) δ 7.71-7.65 (, 4H), 7.64-7.58 (m, 2H), 7.51-7.44 (m, 2H), 7.41-7.25 (m, 6H), 6.98 (s, 1H), 6.84 (s, 1H), 5.44¹ (s(br), 1H), 5.18-5.06 (m, 2H), 5.04-4.93 (m, 2H), 3.07-2.92 (m, 2H), 2.751 (s(br), 3H), 2.02-1.87 (m, 4H), 1.77-1.64 (m, 4H), 1.63-1.52 (m, 2H), 1.31-1.15 (m, 8H), 1.15-0.97 (m, 2H) ¹³C NMR (126 MHz, DMSO_{d6}) δ 171.85, 152.12, 142.64, 140.32, 140.13, 137.60, 137.18, 136.41, 129.42, 129.20, 128.84, 128.10, 127.93, 127.82, 127.53, |
| | | 127.11, 126.96, |
| | | 123.14, 118.14, |
| | | 118.62, 73.51, 66.57, |
| | | 63.97, 52.45, 31.36,¹ |
| | | 29.46, 25.34, 24.44. |
| | | The enantiomeric |
| | | excess was determined |
| | | by HPLC conditions J, |
| | | Tr (major) 8.31 min, |
| | | Tr(minor) 9.81 min. |

¹Average Rotamer Value;
²The absolute stereochemistry of 2v was determined by comparison with literature, see Hanessian, S .; Babonneau, V .; Boyer, N .; Mannoury la Cour, C .; Millan, M. J.; De Nanteuil, G. Design and synthesis of potential dual NK1/NK3 receptor antagonists Bioorg. Med. Chem. Lett. 2014, 24, 510-514. (S)-(Benzyloxycarbonyl-methyl-amino)-phenyl-acetic acid: +112.7 (c 1, CHCl₃).

1. A method of preparing an enantiomerically enriched compound of Formula I: 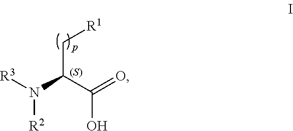
or a salt or solvate thereof,
wherein,
R¹ is selected from the group consisting of: C₃₋₆ cycloalkyl; C₂₋₆ heterocycloalkyl having 1, 2, or 3 heteroatoms selected from O, S, and N; C₆₋₁₀ aryl; and 5- to 10-membered heteroaryl having 1, 2, or 3 heteroatoms selected from O, S, and N; wherein each can be unsubstituted or substituted with one, two, three, or four substituents independently selected from the group consisting of hydroxyl, —O-PG, —O—C₁₋₆ alkyl, halo, and C₁₋₆ alkyl,
wherein PG is an oxygen protecting group, and
wherein the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
wherein R^{x} and R^{y} are each independently selected from the group consisting of hydrogen, C₁₋₆ alkyl, and a N-protecting group;
R² is a carbamate N-protecting group;
R³ is C₁₋₆ alkyl; and
p is 1 or zero;
the method comprising:
contacting in an aqueous solution an enantiomeric mixture of Formula I-i: 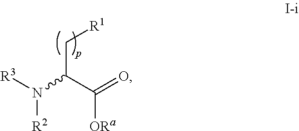
wherein,
R¹, R², R³, and p are as defined in the compound of Formula I; and
R^{a} is C₁₋₆ alkyl or —(CH₂CH₂O)_{q}—C₁₋₆ alkyl, wherein q is 1, 2, 3, 4, or 5;
with a carboxylic ester hydrolase to produce the enantiomerically enriched compound of Formula I, or a salt or solvate thereof.

2. The method of claim 1, wherein R³ is methyl.

3. The method of claim 1, wherein R² is selected from the group consisting of Fmoc, Boc, and Cbz.

4. The method of claim 3, wherein R² is Cbz.

5-7. (canceled)

8. The method of claim 1, wherein R^{a} is —(CH₂CH₂O)_{q}—CH₃.

9-11. (canceled)

12. The method of claim 1, wherein the carboxylic ester hydrolase is selected from the group consisting of Fluka, acylase from Streptomyces toyocaensis, 94734; Sorachim SA, cholesterol esterase from microorganism, COE-313; Toyobo Co., Ltd., lipoprotein lipase from Pseudomonas sp., LIP-301 (immobilized); Toyobo Co., Ltd., lipoprotein lipase from Pseudomonas sp., LPL-311; Sarochim SA, cholesterol esterase from Pseudomonas sp., COE-311; Amano Enzyme USA Co., Ltd., esterase from Escherichia coli, CES-E2; and Lipase 147, Evoxx Technologies GmbH, evo-1.3.147.S.

13. The method of claim 12, wherein the carboxylic ester hydrolase is Lipase 147, Evoxx Technologies GmbH, evo-1.3.147.S.

14-25. (canceled)

26. The method of claim 1, wherein the aqueous solution has a pH from about 8.0 to about 11.0.

27-44. (canceled)

45. The method of claim 1, wherein the compound of Formula I, or a salt or solvate thereof, has an enantiomeric excess of at least 80%.

46-54. (canceled)

55. The method of claim 1, wherein R¹ is: 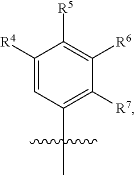
wherein, R⁴, R⁵, R⁶ and R⁷, are each independently selected from the group consisting of hydrogen, hydroxyl, —O-PG, —O—C₁₋₆ alkyl, halo, and C₁₋₆ alkyl,
wherein PG is an oxygen protecting group, and
wherein, the C₁₋₆ alkyl and —O—C₁₋₆ alkyl, in each instance, can be unsubstituted or substituted with one, two, or three substituents independently selected from the group consisting of hydroxy, halo, and —NR^{x}R^{y},
wherein, R^{x} and R^{y} are each independently selected from the group consisting of hydrogen, C₁₋₆ alkyl, and a N-protecting group.

56-83. (canceled)

84. The method of claim 1, wherein the enantiomeric mixture of Formula I-i and the compound of Formula I, or a salt or solvate thereof, are: 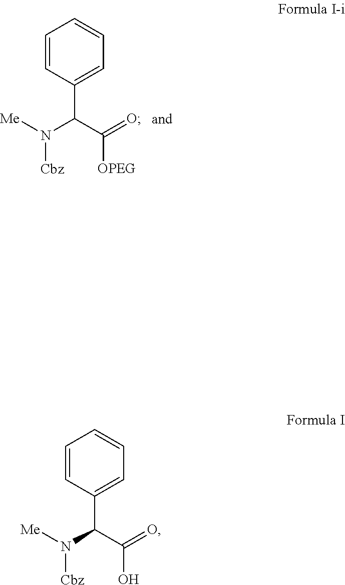
or a salt or solvate thereof; or, 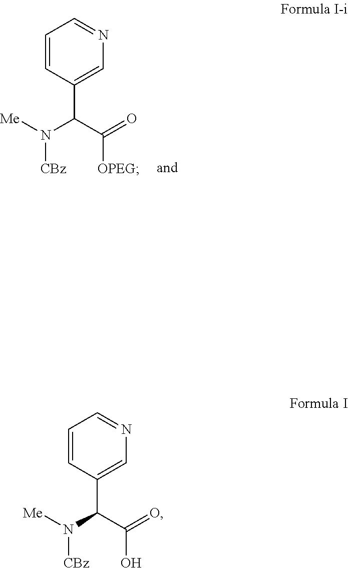
or a salt or solvate thereof; or, 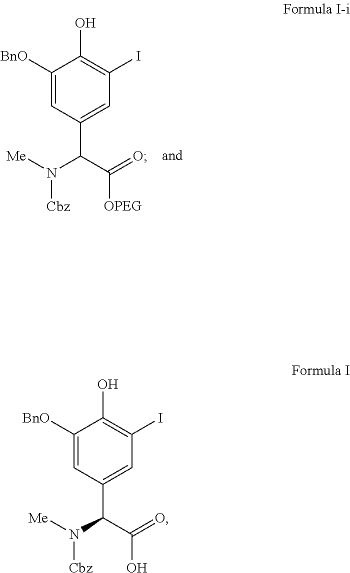
or a salt or solvate thereof; or, 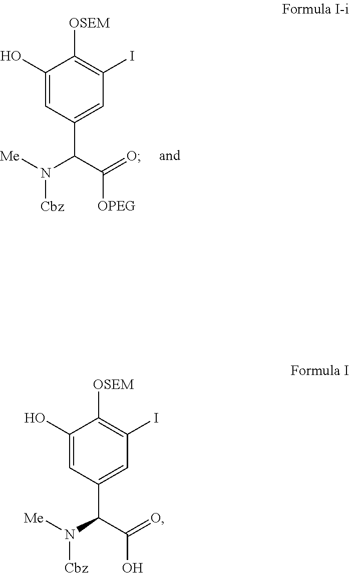
or a salt or solvate thereof; or, 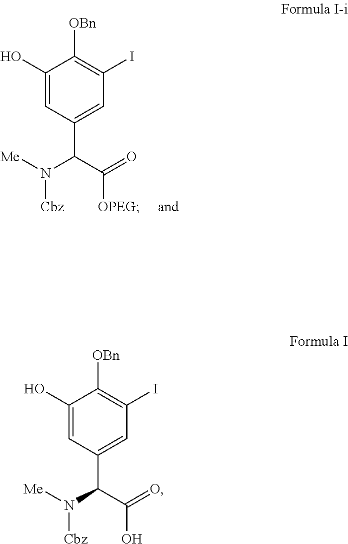
or a salt or solvate thereof; or, 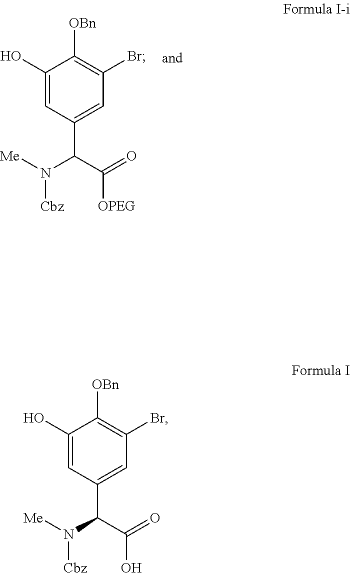
or a salt or solvate thereof; or, 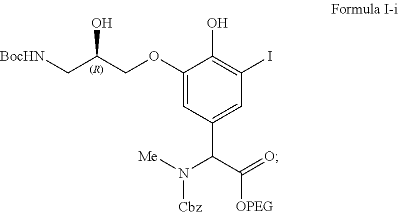
and Formula I is 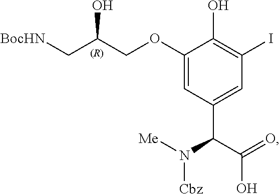
or a salt or solvate thereof; or, 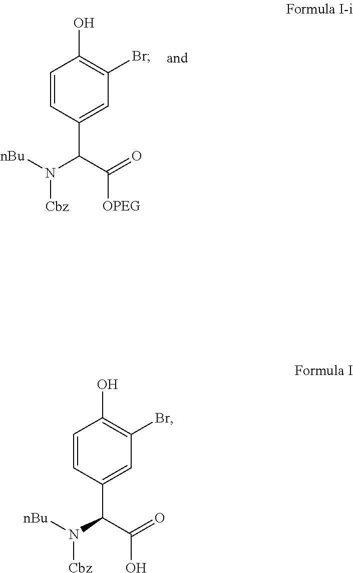
or a salt or solvate thereof; or, Formula I-i is 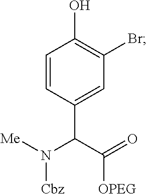
and Formula I is 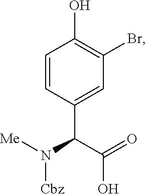 [SMILES: CN(C(=O)OCc1ccccc1)[C@H](C(=O)O)c1ccc(O)c(Br)c1]
or a salt or solvate thereof; or,
Formula I-i is 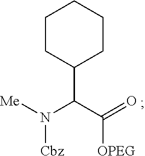
and Formula I is 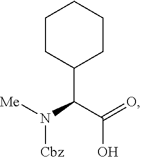 [SMILES: CN(C(=O)OCc1ccccc1)[C@H](C(=O)O)C1CCCCC1]
or a salt or solvate thereof; or,
Formula I-i is 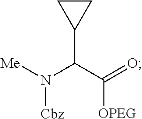
and Formula I is 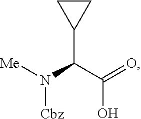 [SMILES: CN(C(=O)OCc1ccccc1)[C@H](C(=O)O)C1CC1]
or a salt or solvate thereof; or,
Formula I-i is 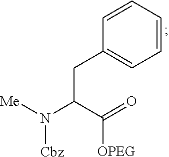
and Formula I is 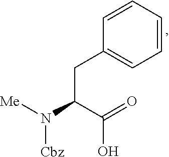 [SMILES: CN(C(=O)OCc1ccccc1)[C@@H](Cc1ccccc1)C(=O)O]
or a salt or solvate thereof; or,
Formula I-i is 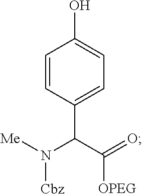
and Formula I is 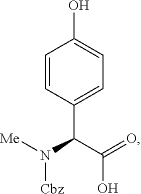 [SMILES: CN(C(=O)OCc1ccccc1)[C@H](C(=O)O)c1ccc(O)cc1]
or a salt or solvate thereof; or,
Formula I-i is 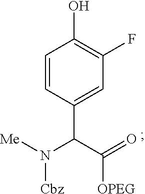
and Formula I is 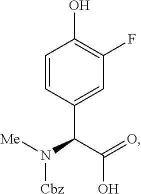 [SMILES: CN(C(=O)OCc1ccccc1)[C@H](C(=O)O)c1ccc(O)c(F)c1]
or a salt or solvate thereof; or,
Formula I-i is 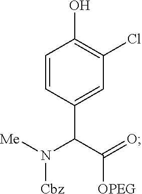
and Formula I is 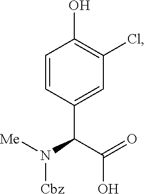 [SMILES: CN(C(=O)OCc1ccccc1)[C@H](C(=O)O)c1ccc(O)c(Cl)c1]
or a salt or solvate thereof; or,
Formula I-i is 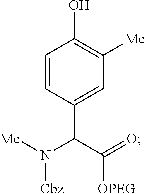
and Formula I is 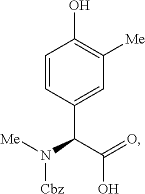 [SMILES: Cc1cc([C@@H](C(=O)O)N(C)C(=O)OCc2ccccc2)ccc1O]
or a salt or solvate thereof; or,
Formula I-i is 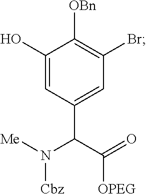
and Formula I is 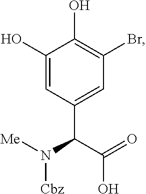 [SMILES: CN(C(=O)OCc1ccccc1)[C@H](C(=O)O)c1cc(O)c(O)c(Br)c1]
or a salt or solvate thereof; or,
Formula I-i is 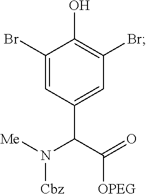
and Formula I is 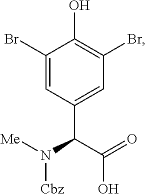 [SMILES: CN(C(=O)OCc1ccccc1)[C@H](C(=O)O)c1cc(Br)c(O)c(Br)c1]
or a salt or solvate thereof; or,
Formula I-i is 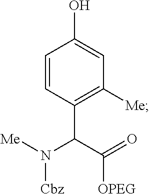
and Formula I is 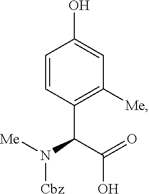 [SMILES: Cc1cc(O)ccc1[C@@H](C(=O)O)N(C)C(=O)OCc1ccccc1]
or a salt or solvate thereof; or,
Formula I-i is 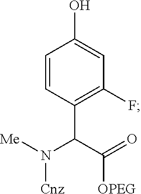
and Formula I is 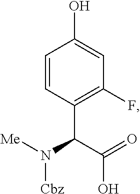 [SMILES: CN(C(=O)OCc1ccccc1)[C@H](C(=O)O)c1ccc(O)cc1F]
or a salt or solvate thereof; or,
Formula I-i is 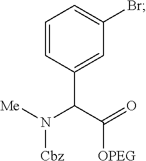
and Formula I is 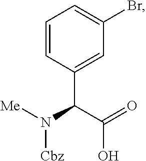 [SMILES: CN(C(=O)OCc1ccccc1)[C@H](C(=O)O)c1cccc(Br)c1]
or a salt or solvate thereof; or,
Formula I-i is 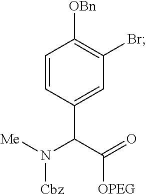
and Formula I is 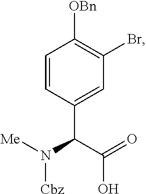 [SMILES: CN(C(=O)OCc1ccccc1)[C@H](C(=O)O)c1ccc(OCc2ccccc2)c(Br)c1]
or a salt or solvate thereof; or,
Formula I-i is 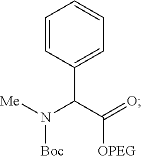
and Formula I is 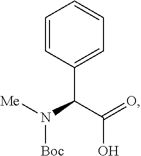 [SMILES: CN(C(=O)OC(C)(C)C)[C@H](C(=O)O)c1ccccc1]
or a salt or solvate thereof;
wherein, in each instance, PEG is —(CH₂CH₂—O)_{q}-Me, wherein q is 1, 2, or 3.

85. (canceled)

86. The method of claim 1, wherein the enantiomeric mixture of Formula I-i and the compound of Formula I, or a salt or solvate thereof, are: 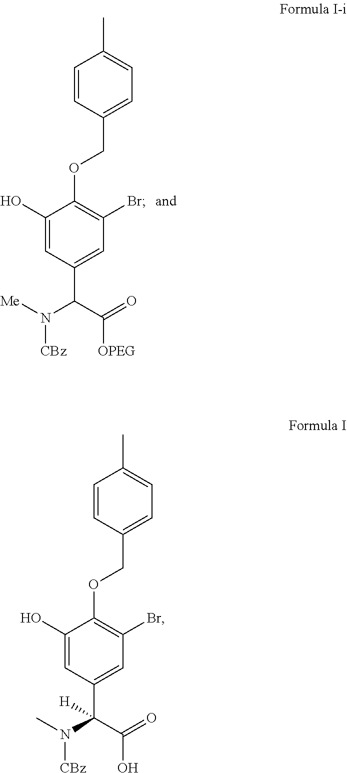
or a salt or solvate thereof;
wherein PEG is —(CH₂CH₂—O)_{q}—CH₃, wherein q is 1, 2, or 3.

87. The method of claim 1, wherein the enantiomeric mixture of Formula I-i and the compound of Formula I, or a salt or solvate thereof, are: 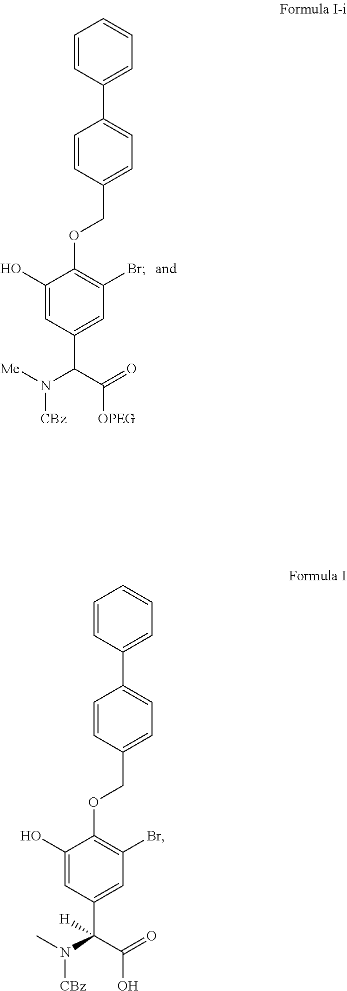
or a salt or solvate thereof;
wherein PEG is —(CH₂CH₂—O)_{q}—CH₃, wherein q is 1, 2, or 3.

88-90. (canceled)

91. The method of claim 1, wherein the enantiomeric mixture of Formula I-i and the compound of Formula I, or a salt or solvate thereof, are: 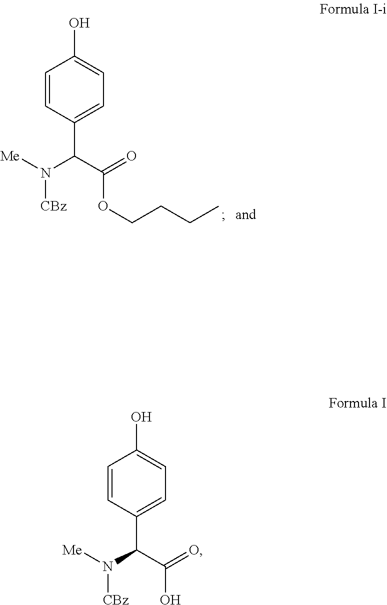
or a salt or solvate thereof; or, 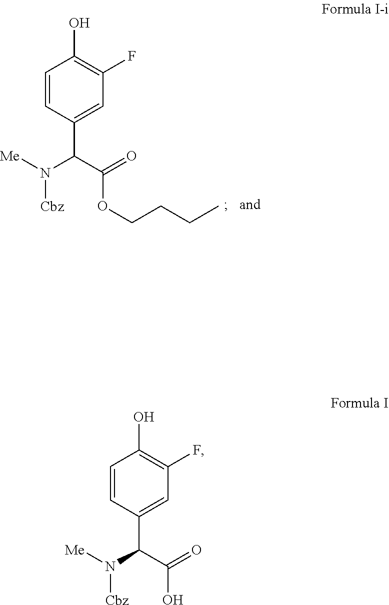 [SMILES: CCCCOC(=O)C(c1ccc(O)c(F)c1)N(C)C(=O)OCc1ccccc1 ; CN(C(=O)OCc1ccccc1)[C@H](C(=O)O)c1ccc(O)c(F)c1]
or a salt or solvate thereof; or, 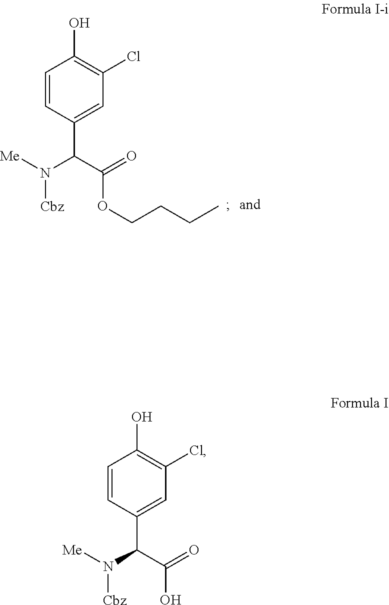 [SMILES: CCCCOC(=O)C(c1ccc(O)c(Cl)c1)N(C)C(=O)OCc1ccccc1 ; CN(C(=O)OCc1ccccc1)[C@H](C(=O)O)c1ccc(O)c(Cl)c1]
or a salt or solvate thereof; or, 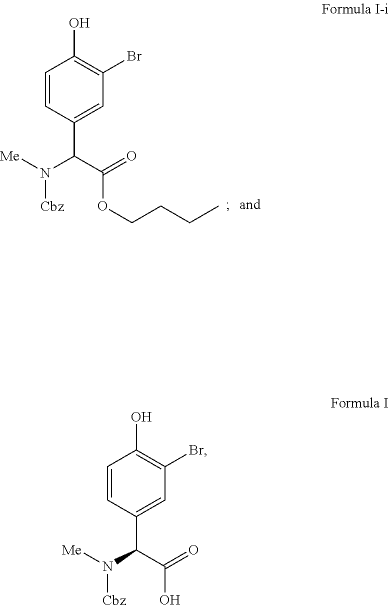 [SMILES: CCCCOC(=O)C(c1ccc(O)c(Br)c1)N(C)C(=O)OCc1ccccc1 ; CN(C(=O)OCc1ccccc1)[C@H](C(=O)O)c1ccc(O)c(Br)c1]
or a salt or solvate thereof; or, 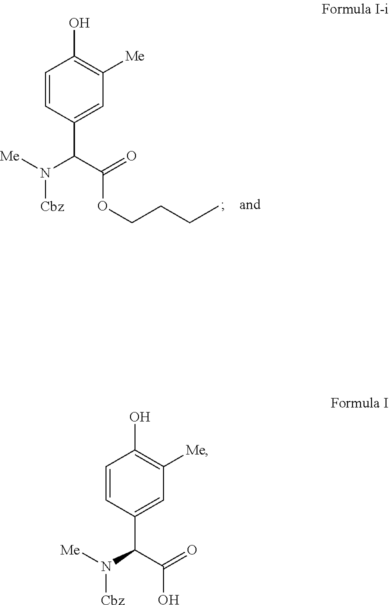 [SMILES: CCCCOC(=O)C(c1ccc(O)c(C)c1)N(C)C(=O)OCc1ccccc1 ; Cc1cc([C@@H](C(=O)O)N(C)C(=O)OCc2ccccc2)ccc1O]
or a salt or solvate thereof; or, 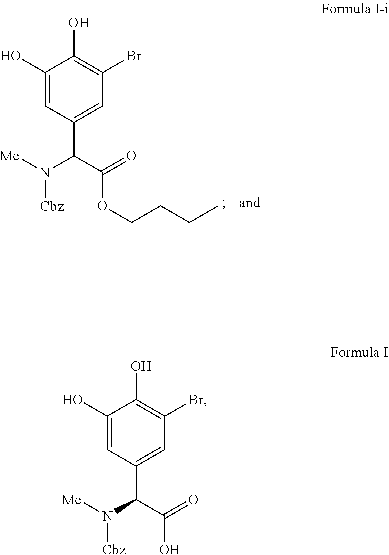 [SMILES: CCCCOC(=O)C(c1cc(O)c(O)c(Br)c1)N(C)C(=O)OCc1ccccc1 ; CN(C(=O)OCc1ccccc1)[C@H](C(=O)O)c1cc(O)c(O)c(Br)c1]
or a salt or solvate thereof; or, 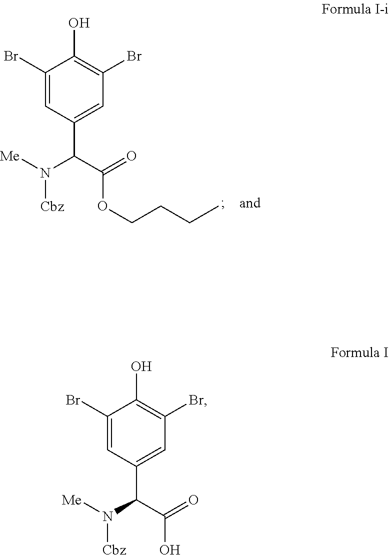 [SMILES: CCCCOC(=O)C(c1cc(Br)c(O)c(Br)c1)N(C)C(=O)OCc1ccccc1 ; CN(C(=O)OCc1ccccc1)[C@H](C(=O)O)c1cc(Br)c(O)c(Br)c1]
or a salt or solvate thereof; or, 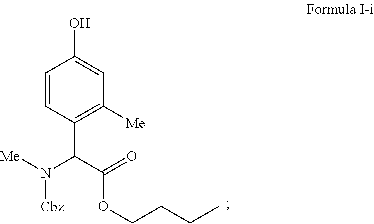 [SMILES: CCCCOC(=O)C(c1ccc(O)cc1C)N(C)C(=O)OCc1ccccc1]
and Formula I is 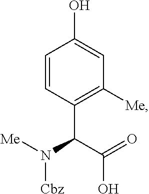 [SMILES: Cc1cc(O)ccc1[C@@H](C(=O)O)N(C)C(=O)OCc1ccccc1]
or a salt or solvate thereof; or, 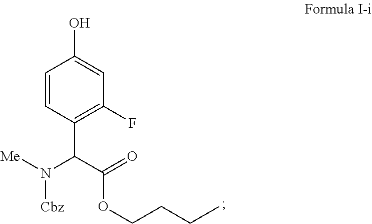 [SMILES: CCCCOC(=O)C(c1ccc(O)cc1F)N(C)C(=O)OCc1ccccc1]
and Formula I is 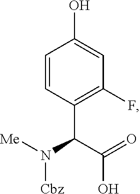 [SMILES: CN(C(=O)OCc1ccccc1)[C@H](C(=O)O)c1ccc(O)cc1F]
or a salt or solvate thereof; or, 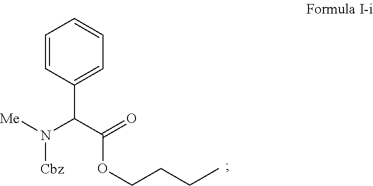 [SMILES: CCCCOC(=O)C(c1ccccc1)N(C)C(=O)OCc1ccccc1]
and Formula I is 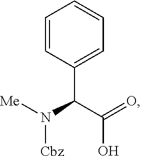 [SMILES: CN(C(=O)OCc1ccccc1)[C@H](C(=O)O)c1ccccc1]
or a salt or solvate thereof; or, 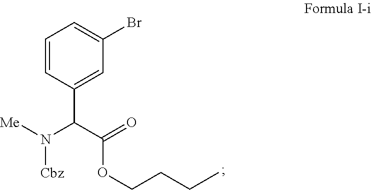 [SMILES: CCCCOC(=O)C(c1cccc(Br)c1)N(C)C(=O)OCc1ccccc1]
and Formula I is 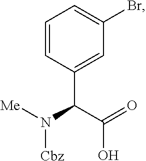 [SMILES: CN(C(=O)OCc1ccccc1)[C@H](C(=O)O)c1cccc(Br)c1]
or a salt or solvate thereof; or, 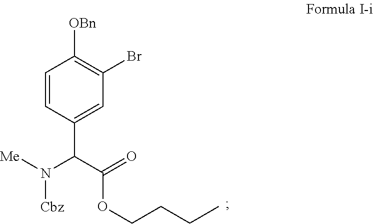 [SMILES: CCCCOC(=O)C(c1ccc(OCc2ccccc2)c(Br)c1)N(C)C(=O)OCc1ccccc1]
and Formula I is 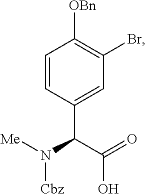 [SMILES: CN(C(=O)OCc1ccccc1)[C@H](C(=O)O)c1ccc(OCc2ccccc2)c(Br)c1]
or a salt or solvate thereof.

92. The method of claim 1, wherein the enantiomerically enriched compound of Formula I is: 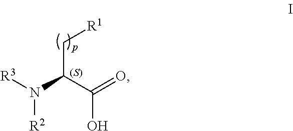
or a salt or solvate thereof,
wherein,
R¹ is selected from the group consisting of: C₃₋₆ cycloalkyl and phenyl, wherein each can be unsubstituted or substituted with one, two, three, or four substituents independently selected from the group consisting of hydroxyl, fluoro, chloro, bromo, and methyl;
R² is a carbamate N-protecting group;
R³ is methyl; and
p is 1 or zero;
the method comprising:
contacting in an aqueous solution, wherein the aqueous solution comprises PEG400, at a temperature of about 45° C., an enantiomeric mixture of Formula I-i: 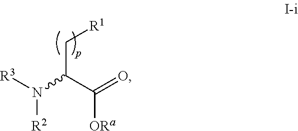
wherein,
R¹, R², R³, and p are as defined in the compound of Formula I; and
R^{a} is n-butyl or —(CH₂CH₂O)_{q}-Me, wherein q is 2 or 3;
with a solution of Lipase 147, Evoxx Technologies GmbH, evo-1.3.147.S dissolved in aqueous glycine buffer at a pH from about 9.0 to about 10.0, to produce the enantiomerically enriched compound of Formula I, or a salt or solvate thereof.

93-99. (canceled)

100. The method of claim 1, wherein the contacting is under dynamic kinetic resolution conditions.

101-109. (canceled)

110. A lysine salt of compound 2ac: 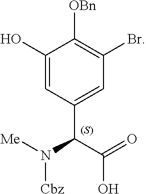
or a piperazine salt of compound 2ac: 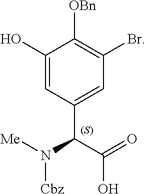
or a dicyclohexylamine salt of compound 2ac:

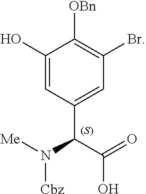

111-139. (canceled)

140. A dicyclohexylamine salt of compound 2ag: 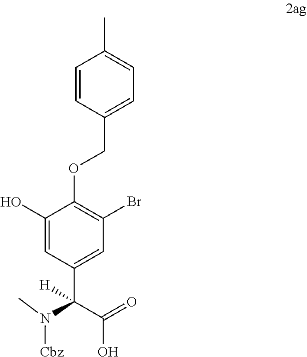 [SMILES: Cc1ccc(COc2c(O)cc([C@@H](C(=O)O)N(C)C(=O)OCc3ccccc3)cc2Br)cc1]
or a dicyclohexylamine salt of compound 2ah:

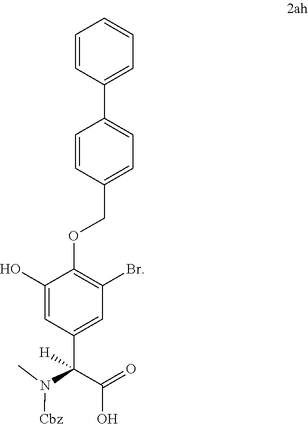 [SMILES: CN(C(=O)OCc1ccccc1)[C@H](C(=O)O)c1cc(O)c(OCc2ccc(-c3ccccc3)cc2)c(Br)c1]

141-145. (canceled)
